(19) United States
(12) Patent Application Publication
(10) Pub. No.: US 2021/0113617 A1
(43) Pub. Date: Apr. 22, 2021

(54) METHODS AND COMPOSITIONS FOR TH9 CELL MEDIATED CANCER TREATMENT

(71) Applicants: Wake Forest University Health Sciences, Winston-Salem, NC (US); The Cleveland Clinic Foundation, Cleveland, OH (US)

(72) Inventors: Yong Lu, Lewisville, NC (US); Qing Yi, Houston, TX (US)

(21) Appl. No.: 17/047,497

(22) PCT Filed: Apr. 17, 2019
(86) PCT No.: PCT/US2019/027815

§ 371 Date: Oct. 14, 2020

Related U.S. Application Data
(60) Provisional application No. 62/658,792, filed on Apr. 17, 2018.

(51) Int. Cl. A61K 35/17; A61P 35/00; A61K 35/15; A61K 39/00; C07K 16/28; C07K 14/725; C07K 14/705; A61K 39/395; A61K 38/17; C12N 5/0783; A61K 31/675

(52) U.S. Cl. CPC A61K 35/17; A61K 2039/5156; A61K 35/15; A61K 39/0011; C07K 16/2803; C07K 14/7051; C07K 14/70521; C07K 14/70578; A61K 39/3955; A61K 38/1774; A61K 38/177; C12N 5/0636; A61K 31/675; A61K 2039/5154; C12N 2501/2304; C12N 2501/2312; C12N 2501/2302; C12N 2501/2306; C12N 2501/2301; C12N 2501/2323; C12N 2501/2321; C12N 2501/15; C07K 2317/73; C07K 2319/30; C07K 2319/33; A61K 2039/505; A61K 2039/572; A61P 35/00

(57) ABSTRACT

The present invention provides methods and compositions for Th9-cell mediated cancer therapy.

### RELATED APPLICATION INFORMATION

This application claims the benefit of U.S. Provisional Patent Application Ser. No. 62/658,792, filed Apr. 17, 2018, the disclosure of which is incorporated herein by reference in its entirety.

Statement Regarding Electronic Filing of a Sequence Listing A Sequence Listing in ASCII text format, submitted under 37 C.F.R. § 1.821, entitled 9151-238WO_ST25.txt, 18,782 bytes in size, generated on Apr. 17, 2019 and filed via EFS-Web, is provided in lieu of a paper copy. This Sequence Listing is incorporated by reference into the specification for its disclosures.

### STATEMENT OF GOVERNMENT SUPPORT

This invention was made with government support under CA190910 awarded by National Institutes of Health. The government has certain rights to the invention.

### FIELD OF THE INVENTION

This invention describes compositions and methods for Th9 cell mediated cancer treatment.

### BACKGROUND OF THE INVENTION

Adoptive cell therapy (ACT) using tumor-specific T cells has focused primarily on CD8⁺ cytotoxic T lymphocytes (CTLs). However, treatment of large established tumors with CD8⁺ CTLs expanded ex vivo has only yielded limited promising results, and systemic administration of IL-2, which is required for survival of effector CD8⁺ T cells, may inhibit infiltration of transferred T cells into tumor tissues and stimulate suppressive effects of regulatory T (Treg) cells.

Although CD8⁺ T cells are potent mediators of antitumor immunity, the role of CD4⁺ T cells in tumor immunity remains underappreciated. Current advances in ACT also suggest that T cells with an early memory and/or a stem cell-like phenotype (Th17 paradigm) and reduced cytolytic function in vitro outperform their short-lived, terminal/end effector-like counterparts (Th1 paradigm) in vivo. Thus, identification of CD4⁺ T cell subsets that possess a mature effector and less exhausted phenotype, and persist significantly longer remains a critical challenge to advancing cancer immunotherapy.

The present invention overcomes previous shortcomings in the art by providing methods and compositions for Th9 cell-mediated cancer therapy.

### BRIEF DESCRIPTION OF THE DRAWINGS

FIGS. 1A-E: Transfer of tumor-specific Th9 cells eradicates the large established tumor. (1A) OVA-specific Th1, Th9 or Th17 cells (CD45.1⁺, 2.5×10⁶) were transferred i.v. into CD45.2⁺ B6 mice bearing 10-day large established B16-OVA tumors (1×10⁶ B16-OVA cells challenged s.c. 10 days before T cell transfer). Adjuvant cyclophosphamide (CTX, i.p.) and DC vaccination (2.5×10⁵, i.v.) were administered to some mice as indicated. (1B) Tumor responses to OT-II T cell transfer are shown (n=5/group). (1C) TRP-1-specific Th1, Th9 or Th17 cells (CD45.2⁺, 2.5×10⁶) were transferred i.v. into CD45.1⁺ B6 mice bearing 10-day large established B16 (1×10⁶ B16 cells challenged s.c. 10 days before T cell transfer). Adjuvant cyclophosphamide (CTX, i.p.) and DC vaccination (2.5×10⁵, i.v.) were administered to some mice as indicated. (1D) Representative tumor responses to TRP-1 T cell transfer are shown (n=5/group). The description of tumor-free survival is summarized from several independent studies. (1E) Tumor responses to OT-II T cell transfer are shown (n=5/group). WT or Cd8α^{-/-} mice received CTX and DC vaccination and transfer of WT, Il9^{-/-} or Ifng^{-/-}Th9 cells. Control mice received no Th9 transfer. Representative results of one from at least two repeated experiments are shown (total mice/group ≥10). Data are mean±SD; *p<0.05, compared with Th17 cells.

FIGS. 2A-I: Th9 cells are distinct cytolytic effector T cells. Mice were treated as shown in FIG. 1A. CD45.1⁺ OT-II Th cells were sorted from the spleens of tumor-bearing mice 12 days after the transfer. RNA (biological samples from two mice) was extracted for gene microarray. (2A) Global transcriptional profiles revealed by microarray of purified Th cell-derived cells from spleens of tumor-bearing mice 12 days after transfer. The heat map shows the log₂-fold change relative to the global average of the top upregulated and downregulated genes, with a cutoff of change in expression >1.5-fold and a p value <0.05. (2B-2D) Heat maps illustrating the relative expression of gene sets as indicated (data are log scaled). (2E) GSEA of the mature effector gene signature. NES, normalized enrichment score; FDR, false discovery rate. (2F-2G) In vitro (5-day) cultured OT-II-Th cells were stained for Eomes expression by FACS. Summarized data for Eomes⁺ cells is shown in (G). (2H) RT-PCR results for expression of the indicated genes in Th cells before transfer (5-day culture, n=3 mice). (2I) Specific killing assay of OT-II-Th cells before transfer (5-day culture) or CD45.1⁺ OT-II Th cells sorted from spleens of tumor-bearing mice (n=3) was performed against B16-OVA cells. Representative results from one of two repeated experiments are shown. Data are mean ±SD; *p<0.05, compared with Th1 or Th17 cells.

FIGS. 3A-G: Th9 cells are a less exhausted effector with long-term persistence capacity. Mice were treated as shown in FIG. 1A. (3A) Heat map illustrating the relative expression of genes (data are log scaled). (3B-3C) Expression of indicated exhaustion markers by transferred cells 12 days after transfer was determined by FACS (gated on CD45.1⁺CD4⁺ cells). Representative data (B) and summarized results (C) are shown. *p<0.05, compared with Th9 or Th17 cells. (3D) GSEA was performed to compare exhaustion-associated gene (down-regulated [top] or up-regulated [bottom]) enrichment in Th1 or Th9 cells. (3E) RT-PCR for expression of the indicated genes. Shown are the relative log 2 expression of selected differentially expressed genes encoding phenotypic markers of terminal differentiation and end-effector function in Th-derived cells recovered from spleens 12 days after transfer (n=3 mice/group). (3F) FACS analysis of the presence of transferred Th cells in tumor-draining lymph nodes (TDLNs) and spleens of mice 10 days after transfer. Representative data are shown. (3G) The relative ratio of transferred cells (CD45.1⁺CD4⁺ cells) to endogenous CD4⁺ cells (CD45.2⁺CD4⁺ cells) summarized from (F) (n=3 mice/group). Representative results of one from two repeated experiments are shown. Data are mean±SD; *p<0.05, compared with Th1 cells.

FIGS. 4A-H: Th9 cells do not acquire a gene signature associated with early memory or stem cell-like feature. Mice were treated as shown in FIG. 1A. (4A) Heat map illustrating the relative expression of genes that have been reported in the literature to be associated with T cell memory subsets (data are log scaled). (4B) GSEA of the early memory gene signature. (4C) Heat map illustrating the relative expression of genes that have been reported in the literature to be associated with self-renewal and hematopoietic stem cell maintenance (data are log scaled). (4D-4E) FACS analysis of apoptotic transferred Th cells (gated on CD45.1⁺CD4⁺ cells) in TDLNs and spleens of tumor-bearing mice (n=3/group) 12 days after transfer. Representative data (D) and summarized results for annexin V⁺ cells (E) are shown. (4F-4G) FACS analysis of apoptotic Th cells (polarized in vitro for 5 days, n=3 mice/group) restimulated with antigen-pulsed antigen-presenting cells (APCs) in vitro. Representative data (F) and summarized results for annexin V⁺ cells (G) are shown. (4H) RT-PCR for expression of the indicated genes. Shown is the relative log 2 expression in Th cells before transfer (polarized in vitro for 5 days, n=3 mice/group) of selected differentially expressed genes encoding phenotypic markers of early memory/stem cell-like T cells. Representative results from one of two repeated experiments are shown. Data are mean SD; *p<0.05, compared with Th1 or Th17 cells.

FIGS. 5A-H: The hyperactivation of late-phase NFκB signaling drives the hyperproliferative feature in Th9 cells. (5A-5B) FACS determination of Ki67⁺ proliferative Th cells (polarized in vitro for 5 days, n=3 mice) restimulated with antigen-pulsed APCs in vitro. Representative flow data (5A) and summarized results (5B) are shown. (5C-5D) Mice were treated as shown in FIG. 1A. FACS analysis of Ki67⁺ proliferating transferred Th cells in TDLNs of tumor-bearing mice (n=3) 12 and 25 days after transfer. Representative data (5C) and summarized results (5D) are shown. *p<0.05, compared with Th9 or Th17 cells. (5E) Naïve CD4⁺ T cells were differentiated for 5-72 hours with plate-bound anti-CD3 mAbs and soluble anti-CD28 mAbs, and NFκB nuclear translocation was analyzed by western blot (nuclear fraction). Solid lines indicate upregulated nuclear translocation of NFκB in Th9 cells. (5F) OT-II-Th cells (polarized in vitro for 5 days) were restimulated with plate-bound αCD3 mAbs and soluble αCD28 mAbs, and NFκB nuclear translocation was analyzed by western blot (nuclear fraction). (5G) OT-II-Th cells (polarized in vitro for 5 days) were labeled with CFSE and cocultured with unpulsed APCs (non-restimulated), OT-II peptide-pulsed APCs (restimulated), or OT-II peptide-pulsed APCs (restimulated) in the presence of QNZ for 48 hours. T alone are Th cells fixed with paraformaldehyde immediately after CFSE labeling. The percentage of CFSE^{low} proliferative cells was determined by FACS. (5H) Th cell yields after the first activation round (day 5, n=3 mice) and after restimulation for an additional 2 days (2^{nd} round, equal number of OT-II-Th cells was collected for the restimulation). QNZ is a specific NFκB inhibitor. Representative results from one of two repeated experiments are shown. Data are mean±SD; *p<0.05, compared with Th1 or Th2 cells.

FIGS. 6A-I: Traf6 is required for the late-phase NFκB hyperactivation in Th9 cells. (6A) Naïve CD4⁺ T cells were differentiated with plate-bound αCD3 mAbs and soluble αCD28 mAbs, and the indicated proteins were analyzed by western blot (cytoplasmic fraction). Red solid lines indicate upregulated NFκB upstream signaling in Th9 cells. (6B) RT-PCR determination of relative Traf6 mRNA expression in OT-II-Th cells polarized in vitro. (6C) WT and Traf6^{-/-} OT-II-Th1, Th9 and Th17 cells were differentiated for 72 hours, and the indicated proteins were analyzed by western blot (cytoplasmic fraction: Traf6, p-IκBα and β-actin; nuclear fraction: p50 and HADC1). (6D) Cell yields of WT and Traf6^{-/-} OT-II-Th1, Th9, and Th17 cells after the first activation round (day 5, n=3 mice). (6E) Luciferase reporter assay for the activation of Traf6 promoter. *p<0.05, compared with control. (6F) ChIP assay of Pu.1 and Stat6 binding to the Traf6 promoter regions in OT-II-Th cells after the first activation round (24 hours, n=3 mice). (6G) ChIP assay for H3K27Ac, H3k4Me1, H3K4Me3 and H3K27Me3 modification of Traf6 loci (enhancer or promoter) in OT-II-Th cells after the first activation round (24 hours, n=3 mice). (6H) OT-II-Th9 cells (24 hours after the first-round activation) were treated with mock, control-vector, Pu.1-shRNA, or Pu.1-expression vector transfection. The indicated molecules were analyzed by western blot 48 hours after treatment (cytoplasmic fraction: Traf6, p-IκBα and β-actin; nuclear fraction: p50 and HADC1). (6I) WT and Stat6^{-/-} OT-II-Th1, Th9 and Th17 cells were differentiated for 72 hours. The indicated molecules were analyzed by western blot 48 hours after treatments (cytoplasmic fraction: Traf6, p-IκBα and β-actin; nuclear fraction: p50 and HADC1). Representative results from one of two repeated experiments are shown. Data are mean SD; *p<0.05, compared with Th1, Th2 or Th17 cells.

FIGS. 7A-G: Traf6 and Eomes dictate the antitumor function of Th9 cells. (7A) WT and Eomes^{-/-} OT-II-Th9 cells were differentiated for 5 days and the relative gene expression was determined by RT-PCR (n=3 mice). (7B) Specific killing assay of WT and Eomes^{-/-} OT-II-Th9 cells before transfer (5-day culture, n=3 mice) was performed against B16-OVA cells. (7C) WT and Eomes^{-/-} OT-II-Th9 cells (CD45.2⁺, 2.5×10⁶) were transferred i.v. into CD45.1⁺ B6 mice bearing B16-OVA tumors (treated similarly to FIG. 1A). Tumor responses are shown (n=5 mice/group). (7D-7G) WT and Traf6^{-/-} OT-II-Th9 cells were differentiated for 5 days (CD45.2⁺, 2.5×10⁶) and transferred i.v. into CD45.1⁺ B6 mice bearing B16-OVA tumors (treated similarly to FIG. 1A). (7D) Representative FACS analysis for the presence of transferred Th9 cells and percentage of Ki67⁺ Th9 cells in spleens of mice 12 days after transfer. (7E) Total number of splenic CD45.2⁺CD4⁺ Th9 cells was calculated from (7D). (F) Percentage of Ki67⁺ cells summarized from (7D). (7G) Tumor responses are shown (n=5 mice/group). Representative results from one of two repeated experiments are shown. Data are mean±SD; *p<0.05.

FIGS. 8A-J: Cytokine profile and antitumor function of Th cells. (8A) Naïve CD4⁺CD62L⁺ T cells were purified from the spleens of OT-II mice and cocultured with irradiated APCs under polarized conditions as detailed in the Methods. Intracellular staining showing the percentages of cytokine-producing cells in polarized Th1, Th9 and Th17 cells. (8B) TRP-1-specific Th1, Th9 or Th17 cells differentiated under different cytokine cocktails in vitro. RT-PCR results for expression of Il17a in Th cells (5-day culture, n=3 mice). (8C) TRP-1 T cells were differentiated for 5 days (CD45.2⁺, 2.5×10⁶) and transferred i.v. into CD45.1⁺ C57BL/6 mice (n=5) bearing 10-day large established B16-OVA tumors. Adjuvant CTX and DC vaccination (2.5×10⁵) were administered. Twelve days later, CD45.2⁺ transferred cells were isolated from spleens of mice and cocultured with unpulsed (non-restimulated) or TRP-1 peptide-pulsed (restimulated) APCs for 48 hours. GM-CSF production in the supernatants was measured by ELISA (n=3). *p<0.05, compared with Th9 or Th17 cells. (8D) OVA-specific Th2 or Th9 cells (CD45.1⁺, 2.5×10⁶) were transferred i.v. into CD45.2⁺ C57BL/6 mice bearing 10-day large established B16-OVA tumors. Adjuvant CTX and DC vaccination (2.5×10⁵) were administered. Tumor responses to OT-I T cell transfer are shown (n=5 mice/group). (8E) TRP-1-specific Th9 cells (CD45.2⁺, 2.5×10⁶) were transferred i.v. into CD45.1+C57BL/6 mice bearing 10-day large established B16 tumors (1×10⁶ B16 cells challenged s.c. 10 days before T cell transfer). Adjuvant cyclophosphamide (CTX, i.p.) with or without DC vaccination (2.5×10⁵, i.v.) were administered to mice as indicated in FIG. 1C. Tumor responses to TRP-1 T cell transfer are shown (n=10-11/group). (8F) TRP-1-specific Th1, Th9 or Th17 cells (CD45.2⁺, 2.5×10⁶), differentiated under different cytokine cocktails, were transferred i.v. into CD45.1+C57BL/6 mice bearing 10-day large established B16 tumors (1×10⁶ B16 cells challenged s.c. 10 days before T cell transfer). Adjuvant cyclophosphamide (CTX, i.p.) and DC vaccination (2.5×10⁵, i.v.) were administered to mice as indicated in FIG. 1C. Tumor responses to TRP-1 T cell transfer are shown (n=5/group). *p<0.05. (8G) Specific killing assay of TRP-1-Th9 cells before transfer (5-day culture) or sorted from spleens of treated mice (n=3, ˜150 days after transfer, from FIG. 1D) was performed using B16 cells as target cells. An E:T ratio of 10:1 was used, and specific killing was determined after 18 hours of coculture. ns: not significant. (8H) TRP-1 Th9 cells (CD45.2⁺, 2.5×10⁶), differentiated for 5 days, were transferred i.v. into CD45.1⁺ C57BL/6 mice (n=9) bearing 10-day large established B16 tumors. Adjuvant CTX and DC vaccinations (2.5×10⁵) were administered. Mice were rechallenged 3 times by s.c. injection of 2.0×10⁶ B16 tumor cells ˜150 days after T cell transfer and then twice more at 1-month intervals. (8I) OVA-specific Th1, Th9 or Th17 cells (CD45.1⁺, 2.5×10⁶) were transferred i.v. into CD45.2⁺ C57BL/6 mice bearing 10-day large established B16-OVA. Adjuvant CTX and DC vaccinations (2.5×10⁵) were administered. FACS analysis of tumor-infiltrating OVA-tetramer-positive, tumor-specific CD8⁺ T cells in tumor tissues of mice 18 days after transfer. Representative (left) and summarized results (right, n=3 mice/group) from (A) are shown. *p<0.05. (8J) OVA-specific Th1, Th9 or Th17 cells (CD45.1⁺, 2.5×10⁶) were transferred i.v. into CD45.2⁺ C57BL/6 mice bearing 10-day large established B16-OVA tumors. Adjuvant CTX and DC vaccinations (2.5×10⁵) were administered. Intracellular staining of IFN-γ producing CD45.1⁺-Th cells in spleens of mice 18 days after transfer. Representative (left) and summarized results (right, n=3 mice/group) from (A) are shown. *p<0.05. Representative results of one from at least two repeated experiments are shown (for antitumor studies in D-H, total number of mice/group ≥10).

FIGS. 9A-F: Features of Th cells after transfer and cytolytic function of Th9 cells. (9A) Naïve OT-II T cells (CD45.2⁺, 2.5×10⁶), differentiated for 5 days, were transferred i.v. into CD45.1⁺ C57BL/6 mice (n=3/group) bearing 10-day large established B16-OVA tumors. Adjuvant CTX and DC vaccinations (2.5×10⁵) were administered. Twelve days later, CD45.2⁺ transferred cells were isolated from spleens of mice and cocultured with unpulsed (non-restimulated) or OT-II peptide-pulsed (restimulated) APCs for 48 hours. Indicated cytokine production in the supernatants was measured by ELISA (n=3). *p<0.05, compared with Th1 or Th17 (up panels), or compared with Th9 (bottom panels). (9B) Naïve OT-II T cells (CD45.1⁺, 2.5×10⁶), differentiated for 5 days, were transferred i.v. into CD45.2⁺ C57BL/6 mice (n=3/group) bearing 10-day large established B16-OVA tumors. Adjuvant CTX and DC vaccinations (2.5×10⁵) were administered. Mice were sacrificed 12 days after transfer, and splenocytes were cultured without or with OT-II peptide restimulation for 24 hours. ICS of transferred CD45.1⁺ T cells was performed to determine the percentage of IL-2-producing cells (n=3 mice). Representative results (left) and summarized data (right, n=3 mice/group) are shown. *p<0.05, compared with control cells. (9C) OVA-specific Th1, Th9 or Th17 cells (CD45.1⁺, 2.5×10⁶) were transferred i.v. into CD45.2⁺ C57BL/6 mice bearing 10-day large established B16-OVA. Adjuvant CTX and DC vaccinations (2.5×10⁵) were administered. FACS analysis of Foxp3⁺ T cells in spleens of mice 18 days after transfer. Cells were pre-gated on CD4⁺ T cells. Representative results (left) and summarized data (right, n=3 mice/group) are shown. *p<0.05, compared with control cells. *p<0.05, compared with Th1 or Th9 cells. (9D) Specific killing assay of OT-II-Th cells (5-day culture, n=3, up panel) was performed against B16-OVA cells with B16 cells as a negative control. An E:T ratio of 10:1 was used, and specific killing was determined after 18 hours of coculture. Inhibition of Th1 and Th9 cell-mediated cytotoxicity against B16 tumor cells by various treatments (bottom panel). Th1 and Th9 cells were preincubated (30 min) with 5 μM Z-AAD-CMK, 20 M DCI, 10 μg/ml anti-FasL mAbs, or the indicated combinations before coculture with B16 cells. An effector/target (E/T) ratio of 10:1 was used. Data are presented as percentage inhibition to Th1 or Th9 cells without treatment (control). (9E) OT-II-Th cells (5-day culture, n=3) were cocultured with unpulsed (non-restimulated) or OT-II peptide-pulsed (restimulated) APCs for 48 hours. Granzyme B and granzyme A production in the supernatants was measured by ELISA (n=3). *p<0.05, compared with Th17. (9F) Naïve OT-II T cells (CD45.1⁺, 2.5×10⁶), differentiated for 5 days, were transferred i.v. into CD45.2⁺ C57BL/6 mice (n=3/group) bearing 10-day established B16-OVA tumors. Adjuvant CTX and DC vaccinations (2.5×10⁵) were administered. Mice were sacrificed 12 days after transfer, and splenocytes were cultured without or with OT-II peptide restimulation for 24 hours. ICS of transferred CD45.1⁺ T cells was performed to determine the percentage of granzyme B-producing cells. Representative results (left) and summarized data (right, n=3 mice/group) are shown. *p<0.05, compared with Th17 cells. Representative results of one from two independent experiments are shown.

FIGS. 10A-D: Effects of long-term persistence of TRP-1 Th17 cells. (10A-10B) Naïve TRP-1 T cells (CD45.2⁺, 2.5×10⁶), differentiated for 5 days, were transferred i.v. into CD45.1⁺ C57BL/6 mice (n=3/group) bearing 10-day large established B16 tumors. Adjuvant CTX and DC vaccination (2.5×10⁵) were administered. (10A) Mice were sacrificed on 18, 45, 65 and ˜150 days after transfer and persistence of transferred Th cells in spleens was determined by FACS. Data are presented as the percentage of transferred TRP-1 cells among total CD4⁺ cells. Summarized data (n=3 mice/group) are shown in (10B). *p<0.05, compared with Th1 cells. (10C-10D) TRP-1 Th9 cells (CD45.2⁺, 2.5×10⁶), differentiated for 5 days, were transferred i.v. into CD45.1⁺ C57BL/6 mice (n=3/group) bearing 10-day large established B16 tumors. Adjuvant CTX and DC vaccinations (2.5×10⁵) were administered. Mice were evaluated 45 days after transfer. (10C) Representative surviving mice are shown (45 days, left). Summarized vitiligo score (right, n>8) of mice treated with Th1, Th9 and Th17 TRP-1 cells on 45 days after transfer. Vitiligo score: 0 (no vitiligo); 1 (vitiligo detected); 2, (>10% vitiligo); 3 (>30% vitiligo); 4 (>50% vitiligo); 5 (>75% vitiligo); 6 (>90% vitiligo); (10D) Eyes from animals analyzed on day 45 after transfer were H&E stained and examined for evidence of autoimmunity in the iris and choroid. Representative images of ocular tissue are shown (left). Summarized ocular autoimmunity score (right, n≥6) of mice treated with Th1, Th9, Th17 TRP-1 cells, or no T cells on 45 days after transfer. The ocular autoimmunity score represents the sum of iridocyclitis, choroiditis, and vitritus using the following scoring method: 0 (none); 1 (mild); 2, (moderate); 3 (severe). *p<0.05. Representative results of one from two independent experiments are shown.

FIGS. 11A-D: Early memory features of Th cells. (11A) Naïve OT-II T cells (CD45.1⁺, 2.5×10⁶), differentiated for 5 days, were transferred i.v. into CD45.2+C57BL/6 mice (n=3/group) bearing 10-day large established B16-OVA tumors. Adjuvant CTX and DC vaccinations (2.5×10⁵) were administered. Mice were sacrificed 12 days after transfer, and splenic transferred CD45.1⁺ T cells were determined for surface expression of the indicated memory/effector markers by FACS (n=3). Representative results (left) and summarized data (right, n=3 mice/group) are shown. *p<0.05, compared with control cells. (11B-11C) TRP-1-Th cells (CD45.2⁺, 2.5×10⁶, polarized in vitro for 5 days) were transferred i.v. into CD45.1+C57BL/6 mice (n=3/group) bearing 10-day large established B16 tumors. Adjuvant CTX and DC vaccination (CD45.1⁺, 2.5×10⁵) were administered. Mice were sacrificed 18, 45 and around 150 days after transfer and splenocytes were stained with annexin V/PI immediately. (B) Representative results (left) and summarized results for annexin V⁺ CD45.2⁺TRP-1 Th cells (right, n=3-4 mice/group) are shown. *p<0.05, compared with Th1 or Th9 cells. (C) The total number of TRP-1 Th cells and apoptotic TRP-1 cells in the spleen (n=3-4 mice/group) were calculated from FACS. *p<0.05, compared with Th1. (11D) Naïve CD4⁺CD62L⁺ T cells were purified from the spleens of TRP-1 mice and cocultured with irradiated APCs under polarized conditions as detailed in the Methods (polarized in vitro for 5 days, n=3 mice/group). RT-PCR was performed to determine the expression of the indicated genes. Shown is the heat map illustrating the relative expression of genes (data are log scaled) of selected differentially expressed genes encoding phenotypic markers of terminally differentiated end-effector and early memory/stem cell-like T cells. Representative results from one of two repeated experiments are shown.

FIGS. 12A-F: Proliferation and apoptosis of Th cells. (12A-12C) TRP-1 Th9 cells (CD45.2⁺, 2.5×10⁶), differentiated for 5 days, were transferred i.v. into CD45.1⁺ C57BL/6 mice (n=3/group) bearing 10-day large established B16 tumors. Adjuvant CTX and DC vaccinations (2.5×10⁵) were administered. Mice were sacrificed on ˜150 days after transfer and transferred Th cells (CD45.2⁺ cells) in spleens were determined by FACS. (A-B) Splenocytes were stained with Ki67 immediately (non-restimulated) or after overnight culture with TRP-1 peptide (Restimulation). Shown are representative figures (A) and summarized data (B, n=3/group) of the percentage of Ki67⁺ proliferative cells in transferred CD45.2⁺ Th9 cells. (C) Splenocytes were stained for the indicated exhaustion markers. Shown are representative figures (left) and summarized data (right, n=3 mice/group) of the percentage of exhaustion marker-positive cells in transferred CD45.2⁺ Th9 cells. *p<0.05. (12D) Naïve CD4⁺ T cells were differentiated for 0-24 hours with plate-bound anti-CD3 mAbs and soluble anti-CD28 mAbs, and the indicated proteins were analyzed by western blot. Th cells were differentiated for the indicated times (0-3 hrs) and the expression level of the TCR-proximal signal proteins was analyzed by western blot (cytoplasmic fraction, up panel). Th cells were differentiated for 24 hrs and the expression level of the indicated molecules was analyzed by western blot (cytoplasmic fraction, bottom left panel). Th cells were differentiated for 0-24 hrs and the expression level of the indicated proteins was analyzed by western blot (nuclear fraction, bottom right panel). (12E) Apoptosis of Th9 cells in the presence of NF-κB inhibitor. OT-II-Th9 cells 5 (polarized in vitro for 5 days) were restimulated with antigen-pulsed APCs in vitro for 2 days in the presence of QNZ, an NF-κB-specific inhibitor. Percentage of apoptotic cells was determined by FACS with Annexin V/PI staining. Representative (top) and summarized results for annexin V⁺ Th9 cells (bottom, n=3-4 mice/group) are shown. (12F) Effects of the NFκB inhibitor QNZ on the proliferation of Th17 cells. OT-II-Th17 cells (polarized in vitro for 5 days) were labeled with CFSE and restimulated with antigen-pulsed APCs in vitro for 2 days in the presence of QNZ. The percentage of CFSE^{low} proliferative cells was determined by FACS. Representative results of one from two independent experiments are shown.

FIGS. 13A-C: Traf6 upregulation in Th9 cells does not require IL-9 signaling. (13A) Naïve CD4⁺ T cells were differentiated for 24 or 72 hours with plate-bound anti-CD3 mAbs and soluble anti-CD28 mAbs. The indicated proteins were analyzed by western blot (cytoplasmic fraction: Traf6, p-IκBα and β-actin; nuclear fraction: p50 and HADC1). (13B-13C) IL-9 signaling seems not to be required for Traf6 expression or hyperproliferation of Th9 cells. Naïve WT- or Il9r^{-/-}-OT-II-CD4⁺ T cells were polarized in vitro for 3 or 5 days. (13B) Cell yields of Th cells after the first activation round (day 5, n=3 mice). (13C) The indicated proteins were analyzed by western blot 72 hours after polarization (cytoplasmic fraction: Traf6 and β-actin; nuclear fraction: p50 and HADC1). Representative results from one of two repeated experiments are shown.

FIGS. 14A-I: Eomes and Traf6 do not interact in Th9 cells. (14A-14C) Effects of Eomes on the proliferation of Th9 cells. (14A) WT and Eomes^{-/-} OT-II-Th9 cells were differentiated in vitro. RT-PCR was performed for the expression of indicated genes in Th cells (5-day culture, n=3 mice). *p<0.05, compared with Eomes KO-Th9. (14B) OT-II-Th cells (polarized in vitro for 5 days) were labeled 30 with CFSE and restimulated with antigen-pulsed APCs in vitro for 2 days. The percentage of CFSE^{low} proliferative cells was determined by FACS. (14C) Th cell yields (n=3 mice) calculated from (b). *p<0.05, compared with Traf6 KO-Th9. (14D-14F) Effects of Traf6 on the proliferation of Th9 cells. OT-II-Th cells (CD45.2⁺, 2.5×10⁶, polarized in vitro for 5 days) were labeled with CFSE and transferred i.v. into CD45.1⁺ C57BL/6 mice (n≥6/group) bearing 10-day large established B16-OVA tumors. Adjuvant CTX and DC vaccinations (CD45.1⁺, 2.5×10⁵) were administered. (14D) Mice were sacrificed 4 days after transfer, and CFSE intensity of splenic CD45.2⁺ cells (transferred Th cells) and endogenous CFSE negative cells (CD45.1⁺ cells) was accessed by FACS (left, representative histograms overlaid) and also shown as CFSE MFI (mean fluorescence intensity, right, n=3 mice). (14E) Mice were sacrificed 7 days after transfer, and CFSE intensity of splenic CD45.2⁺ cells (transferred Th cells) and endogenous CFSE negative cells (CD45.1⁺ cells) was assessed by FACS (left, representative histograms overlaid) and also shown as CFSE MFI (right, n=3 mice). (14F) Mice were sacrificed 4 days after transfer and stained immediately with annexin V. Shown are annexin V⁺ apoptotic versus CFSE dilution of splenic CD45.2⁺ cells (transferred Th cells) with representative plots (left) and summarized data (right). *p<0.05, compared with all other cells. (14G-14H) Effector function of Traf6^{-/-} Th9. (14G) WT and Traf6^{-/-} OT-II-Th9 cells were differentiated in vitro. RT-PCR was performed for the expression of indicated genes in Th cells (5-day culture, n=3 mice). (14H) Specific killing assay of OT-II-Th cells (5-day culture, n=3) was performed against B16-OVA cells with B16 cells as a negative control. An E:T ratio of 10:1 was used, and specific killing was determined after 18 hours of coculture. *p<0.05, compared with Traf6 KO-Th9. (14I) WT and Traf6^{-/-} OT-II-Th17 cells were differentiated for 5 days (CD45.2⁺, 2.5×10⁶) and transferred i.v. into CD45.1⁺ C57BL/6 mice (n=5/group) bearing 10-day large established B16-OVA tumors. Adjuvant CTX and DC vaccinations (2.5λ10⁵) were administered. Tumor responses to OT-II T cell transfer are shown. Representative results of one from two independent experiments are shown (total number of mice/group=10). *p<0.05. Representative results from one of two repeated experiments are shown.

FIG. 15: Lung cancer model. LL2-OVA cells (1×10⁶ murine lung cancer cells) inoculated into B6 mice. OTT-II Th9 cells (2.5×10⁶) were transferred into mice ten days later. Cyclophosphamide and dendritic cells were also administered to the mice.

FIG. 16: Colon cancer model. Mc38 OVA cells (1×10⁶ murine colon cancer cells) inoculated into B6 mice. OT-II cells (2.5×10⁶) were transferred into mice ten days later. Cyclophosphamide and dendritic cells were also administered to the mice.

FIG. 17: Pancreatic cancer model. Pan02-OVA cells (1×10⁶ murine pancreatic cancer cells) inoculated into B6 mice. OT-II cells (2.5×10⁶) were transferred into mice ten days later. Cyclophosphamide and dendritic cells were also administered to the mice.

FIG. 18: B cell lymphoma model. Raji cells (1×10⁶ human B cell lymphoma) inoculated into NOD scid gamma (NSG) mice. Human CD19CAR T cells (Th9, 3×10⁶); or CD19CAR T cells (Th1, 1.5×10⁶+Tc1, 1.5×10⁶) were transferred into mice seven days later.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted.

In the following description, certain details are set forth such as specific quantities, sizes, etc. so as to provide a thorough understanding of the present embodiments disclosed herein. However, it will be obvious to those skilled in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

The present invention is based on the discovery of a method of treating cancer in a subject in need thereof, comprising administering to the subject an effective amount of a CD4⁺ Th9 cell that has specificity for cancer cells in the subject.

The present invention further provides a method of reducing/eradicating a tumor in a subject in need thereof, comprising administering to the subject an effective amount of a CD4⁺ Th9 cell that has specificity for the tumor in the subject.

According to embodiments of the present invention, a CD4⁺ Th9 cell is provided or a plurality of CD4⁺ Th9 cells (e.g., a population of CD4⁺ Th9 cells). A CD4⁺ Th9 cell of the present invention may have specificity for a cancer cell and/or may be primed (e.g., with a cancer antigen-loaded APC) to have specificity for a cancer cell (e.g., a cancer cell in a subject for which the primed CD4⁺ Th9 cell is to be administered). The CD4⁺ Th9 cell may be programmed as and/or exhibit the phenotype for a mature effector T cell. In some embodiments, the CD4⁺ Th9 cell may exhibit and/or maintain a mature effector cell signature with cytolytic activity as strong as Th1 cells and/or that may persist as long as Th17 cells in vivo. In some embodiments, the CD4⁺ Th9 cell may exhibit cytotoxicity that is at least about 60% or more (e.g., 60%-140% or more) of the cytotoxicity of Th1 cells and/or that is greater than about 100% of the cytotoxicity Th17 cells. Cytolytic activity may be measured using an in vitro and/or in vivo cytolytic assay known to those of skill in the art. In 20 some embodiments, the CD4⁺ Th9 cell may exhibit an expression level of Id2, Eomes, Id3, 112, and/or a granzyme (e.g., Gzma, Gzmb, Gzmd, Gzme, Gzmk, Gzmg, and/or Gzmn) that is similar (e.g., within 10%) and/or increased compared to the expression level of the same gene in a Th1 and/or Th17 cell. The CD4⁺ Th9 cell may be put under conditions to express Id2, Eomes, Id3, 112, and/or a granzyme (e.g., Gzma, Gzmb, Gzmd, Gzme, Gzmk, Gzmg, and/or Gzmn), optionally at an increased level compared to the expression level of the same gene in a Th1 and/or Th17 cell, and/or may be put under conditions to overexpress Id2, Eomes, Id3, 112, and/or a granzyme (e.g., Gzma, Gzmb, Gzmd, Gzme, Gzmk, Gzmg, and/or Gzmn). In some embodiments, the CD4⁺ Th9 cell may exhibit an expression level of Id2, Eomes, Id3, 112, and/or a granzyme (e.g., Gzma, Gzmb, Gzmd, Gzme, Gzmk, Gzmg, and/or Gzmn) that is increased by about 15%, 20%, 30%, 40%, 50%, 60%, 70%, 80%, 90% or more compared to the expression level of the same gene in a Th1 and/or Th17 cell. In some embodiments, the CD4⁺ Th9 cell may exhibit an expression level of Id2, Eomes, Id3, 112, and/or a granzyme (e.g., Gzma, Gzmb, Gzmd, Gzme, Gzmk, Gzmg, and/or Gzmn) that is increased by at least 30% or more compared to the expression level of the same gene in a Th1 and/or Th17 cell. In some embodiments, the CD4⁺ Th9 cell may exhibit an increased expression level (e.g., by at least 30%) of Eomes compared to the expression level of Eomes in a Th1 and/or Th17 cell. The CD4⁺ Th9 cell may not carry and/or may not exhibit the molecular signature of a T cell exhaustion phenotype (such as in a Th1 cell).

The CD4⁺ Th9 cell may express and/or be capable of expressing a hyperproliferative phenotype. “Hyperproliferative” and grammatical variations thereof as used herein in reference to a T cell (e.g., a Th9, Th1, or Th17 cell) refers to the cell expressing Ki67 and a plurality of the T cells (e.g., a Th9 cell population) in which greater than 50% of the cells in the plurality are Ki67⁺ (with Ki67⁺ meaning that the cell(s) expresses Ki67). In some embodiments, the CD4⁺ Th9 cell or a plurality of the CD4⁺ Th9 cells is hyperproliferative. In some embodiments, the percentage of Ki67⁺ cells in the plurality of CD4⁺ Th9 cells is increased compared to the percentage of Ki67⁺ cells in a plurality of Th1 and/or Th17 cells (e.g., a population of similar or comparable size).

The CD4⁺ Th9 cell, upon administration to a subject, may exhibit and/or exert an antitumor response in the subject and the antitumor response may be complete. In some embodiments, the CD4⁺ Th9 cell, upon administration to a subject, reduces or completely eliminates a tumor such as a large established tumor, and there is no tumor relapse for at least about 1, 2, 3, 4, 5, 6, 7, 8, 9, 10, 11, 12 months or more. In some embodiments, the CD4⁺ Th9 cell administered to the subject is and/or upon administration becomes a Ki67⁺ cell. In some embodiments, a plurality of the CD4+Th9 cells comprises at least about 50%, 55%, 60%, 65%, 70%, 75%, 80%, 85%, 90%, 95%, or more CD4⁺ Th9 cells that are Ki67⁺ cells and/or upon administration become Ki67⁺ cells.

A method of the present invention may comprise administering a CD4⁺ Th9 cell of the present invention or a plurality of CD4⁺ Th9 cells of the present invention to a subject. A method of the present invention may further comprise administering to the subject an agent to induce lymphopenia (e.g., cyclophosphamide) and the agent to induce lymphopenia may at least temporarily induce lymphopenia in the subject.

The agent to induce lymphopenia may be administered to the subject prior to, concurrently with, and/or after administration of the CD4⁺ Th9 cell to the subject. A method of the present invention may further comprise administering a vaccine to the subject and the vaccine may be administered to the subject prior to, concurrently with, and/or after administration of the CD4⁺ Th9 cell and/or agent to induce lymphopenia in the subject. In some embodiments, the agent to induce lymphopenia and the vaccine are concurrently administered, optionally about 1 or 2 days or about 20, 18, 12, 6, 4, 2, or 1 hour(s) before administration of the CD4⁺ Th9 cell to the subject. In some embodiments, the vaccine and the CD4⁺ Th9 cell are administered concurrently, optionally in a separate sequential administration. In some embodiments, the vaccine is administered about 1, 2, 4, 6, 12, 18, or 20 hours, or about 1 or 2 days after administration of the CD4⁺ Th9 cell. In some embodiments, the vaccine is a dendritic cell (DC) vaccine such as, but not limited to, a peptide-pulsed DC vaccine, an idiotype-pulsed DC vaccine, and/or a tumor lysate-pulsed DC vaccine. A DC vaccine may be pulsed with a tumor lysate prepared from a tumor in a subject who is to be administered the vaccine and CD4⁺ Th9 cell. In some embodiments, a DC vaccine may be pulsed with an antigen (e.g., a peptide) that is specific for and/or associated with a tumor in a subject who is to be administered the vaccine and CD4⁺ Th9 cell. In some embodiments, the vaccine is a vaccine that increases the antitumor response during adoptive cell therapy (ACT) and/or a vaccine that increases the amount of hyperproliferation for CD4⁺ Th9 cells. In some embodiments, the vaccine comprises antigen-loaded DCs. In some embodiments, the vaccine is a peptide vaccine, which may be in any formula. In some embodiments, the vaccine comprises a vector, cell, and/or virus that encodes an antigen such as a peptide and/or tumor antigen.

According to some embodiments, a method of reducing/eradicating a tumor in a subject in need thereof comprises administering to the subject an effective amount of a CD4⁺ Th9 cell that has specificity for the tumor in the subject, administering to the subject an agent to induce lymphopenia (optionally prior to administering the CD4⁺ Th9 cell), and administering to the subject a vaccine (optionally after administering the CD4⁺ Th9 cell). In some embodiments, only one administration of the CD4⁺ Th9 cell, agent to induce lymphopenia, and/or vaccine is needed to reduce or eradicate the tumor. In some embodiments, the CD4⁺ Th9 cell, agent to induce lymphopenia, and/or vaccine is administered to the subject two or more times (e.g., 2, 3, 4, 5 or more).

In further embodiments, the present invention provides a method of producing a T cell having a hyperproliferation phenotype, comprising introducing into a memory T cell or effector T cell a heterologous nucleotide sequence that encodes Traf6 and/or Eomes under conditions whereby the nucleotide sequence is expressed to produce the Traf6 protein and/or Eomes protein in the cell.

As one nonlimiting example, effector T cells or memory T cells are prepared as follows: Naïve T cells, tumor-infiltrating T cells, and/or T cells isolated from peripheral blood mononuclear cells (PBMCs) are cultured with interleukin-2 (IL-2), interleukin-7 (IL-7), and/or interleukin-15 (IL-15), in any combination. These T cells can be transduced with a viral vector and/or transfected with a nonviral vector or nucleic acid construct encoding nucleotide sequences that encode Traf6 and/or Eomes, and/or stimulated to enhance the expression of endogenous nucleotide sequences encoding Traf6 by OX40L, GITRL, anti-OX40 agonist mAbs, and/or anti-GITR agonist mAbs.

Nonlimiting examples of a nucleotide sequence encoding Eomes include GenBank Accession No. NM_001278182.1, GenBank Accession No. NM_005442.3 and GenBank Accession No. NM_001278183.1, the entire contents of each of which are incorporated by reference herein.

Nonlimiting examples of a nucleotide sequence encoding Traf6 include GenBank Accession No. NM_145803.2 and GenBank Accession No. NM_004620.3, the entire contents of each of which are incorporated by reference herein.

Further provided herein is a cell having a hyperproliferation phenotype, produced by the methods described herein, as well as a method of treating cancer in a subject in need thereof and/or a method of reducing/eradicating a tumor in a subject in need thereof, comprising administering to the subject an effective amount of T cells having a hyperproliferation phenotype produced according to the methods of this invention.

In some embodiments, the methods of this invention can include administration of IL-9 secreting CD8⁺ Tc9 cells to a subject, along with the Th9 cells of this invention. Differentiation of CD8⁺ T cells under T helper 9-polarizing conditions can induce the development of an IL-9 producing CD8⁺ T (Tc9) cell subset which elicits a greater antitumor response against large established tumors than classic type-1 CD8⁺ cytotoxic T cells that are presently used in clinical protocols. Methods of making IL-9 secreting CD8⁺ Tc9 cells are known in the art, including for example, as described in U.S. Pat. No. 9,694,033, the entire contents of which are incorporated by reference herein.

Cells to be employed in the methods and compositions of this invention can be obtained from a subject (e.g., a human subject). In some embodiments, the cells can be from the same subject to whom the treatment(s) will be administered (i.e., the cells are autologous cells). In other embodiments, the cells can be from a subject that is not the same subject to whom the treatment(s) will be administered (e.g., allogeneic cells).

In some embodiments naïve T cells or unselected T cells can be isolated from a blood sample and/or spleen of a subject, such as a donor or recipient subject, using standard methods including, e.g., Ficoll density gradient centrifugation followed by negative selection to remove undesired cells. Methods of isolating naive T cells are known to those of skill in the art and include FACS sorting of cells. Naive T cells or unselected T cells can also be obtained from a subject using an apheresis procedure.

In some embodiments, a population of PBMC, naive T cells or unselected T cells is contacted with an immunogenic peptide, coated or soluble anti-CD3/anti-CD28 mAbs, or anti-CD3/anti-CD28 conjugated beads in order to prime the T cells. An immunogenic peptide for use in the invention can be prepared synthetically, or by recombinant DNA technology or isolated from natural sources such as whole viruses or tumors. The Th9 cells produced are typically specific for an antigen present on a tumor (e.g., a solid tumor). Therefore, in certain embodiments, the immunogenic peptide is isolated or derived from a tumor (e.g., a subject's cancerous solid tumor).

In some embodiments, the desired immunogenic peptide can be loaded into the binding pockets of MHC molecules on the surface of antigen presenting cells (APCs) using standard methods. In some embodiments, the APCs of this invention can be loaded with a total cell or membrane preparation from cancer cells instead of or in combination with a molecularly defined antigen preparation.

In an exemplary embodiment, the APCs are irradiated antigen presenting dendritic cells which become peptide-loaded antigen dendritic cells when loaded with a desired immunogenic peptide. Typically, the antigen presenting cells are irradiated so APCs won't proliferate in response to T cell produced cytokines or other cytokines added to the culture.

In some embodiments, a population of naive T cells or unselected T cells can be genetically engineered to produce receptors on their surface called chimeric antigen receptors (CARs). CARs are proteins that allow the T cells to recognize a specific protein (antigen) on tumor cells (e.g., a solid tumor cell from a subject having cancer). For example, naive T cells can be transfected with and grown to express nucleotide sequences encoding CARs such that T cells producing CARs can target and kill tumors via tumor-associated antigens.

Appropriate means for preparing an engineered population of lymphocytes expressing a selected CAR nucleic acid construct will be well known to the skilled artisan, and can include, for example, retrovirus, lentivirus (viral mediated CAR gene delivery system), sleeping beauty, and/or piggyback (transposon/transposase systems that include a non-viral mediated CAR gene delivery system).

In some embodiments, primed Th9 cells may be effectively separated from the APC using one of a variety of known methods. For example, monoclonal antibodies specific for the APCs, for the peptides loaded onto the stimulator cells, or for Th9 (or a segment thereof) may be utilized to bind the appropriate complementary ligand. Antibody-tagged cells may then be extracted from the admixture via appropriate means, e.g., via fluorescence activated cell sorting (FACS) protocols and/or magnetic bead separation protocols as are known in the art.

The cultures described herein can typically be incubated under conditions of temperature and the like that are suitable for the growth and differentiation of T lymphocytes. For the growth of human T lymphocytes, for example, the temperature will generally be at least about 25 Celsius, and in some embodiments, at least about 30°, and in some embodiments, about 37° C.

The administration of a pharmaceutical composition including Th9 cells may be for either “prophylactic” or “therapeutic” purpose. When provided prophylactically, therapy can be provided in advance of any symptom. The prophylactic administration of the therapy serves to prevent development of cancer. Prophylactic administration may be given to a subject “in need thereof,” which can be a subject that is at risk of cancer due to, for example, a family history of cancer, or a previous cancer episode. Alternatively, the Th9 cells may be given to a subject with changing (e.g., rising) cancer marker levels. Multiple biomarkers for particular cancers are known in the art. For example, melanoma markers are described in PCT Publications WO 2008/141275, WO 2009/073513, or in U.S. Pat. No. 7,442,507.

Methods for administering cells are well known to those of skill in the art, including, e.g., as described in WO 2004/048557; WO 2008/033403; U.S. 2008/0279813 WO2008/033403; U.S. Pat. No. 7,572,631; and WO 2009/131712, which are all herein incorporated by reference in their entirety. The amount of Th9 cells that will be effective in the treatment and/or suppression of cancer may be determined by standard clinical techniques. The dosage will depend on the type of cancer to be treated, the severity and course of the cancer, previous therapy the recipient has undergone and/or is undergoing, the recipient's clinical history, and the discretion of the attending physician. The Th9 cell population may be administered in various treatment regimens, e.g., a single or a few doses over one to several days to ameliorate symptoms and/or periodic doses over an extended time to inhibit cancer progression or to prevent cancer recurrence. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

The methods described herein are useful for the treatment of any type of cancer in a subject. As used herein, the term “cancer” includes any type of cancer. A “cancer” in a subject refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within a subject, or may be a non-tumorigenic cancer cell, such as a leukemia cell.

In some embodiments, in a method of the present invention, the subject has received and/or is receiving one or more than one (e.g., 2, 3, 4, 5, etc.) agent to induce lymphopenia, which can be a temporary lymphopenia. Nonlimiting examples of an agent to induce lymphopenia according to the methods of this invention include cyclophosphamide, bendamustine, fludarabine, total body irradiation, and any combination thereof. In some embodiments, an agent to induce lymphopenia (e.g., cyclophosphamide) is administered to a subject prior to, concurrently with, and/or after a Th9 cell of the present invention is administered to a subject. For example, in some embodiments, an agent to induce lymphopenia is administered to a subject about 1 or 2 days or about 20, 18, 12, 6, 4, 2, or 1 hour(s) before administration of a Th9 cell of the present invention to the subject. An agent to induce lymphopenia may be administered in an amount sufficient to induce lymphopenia (e.g., temporary lymphopenia), and optionally the agent may be administered in an amount of about 25, 50, 100, or 150 mg/kg to about 200, 250, 300, 400, or 500 mg/kg (mg of the agent per kg of the subject).

In additional embodiments, the methods of this invention can further comprise the step of administering to the subject an effective amount of a vaccine such as, but not limited to, a cancer antigen-loaded antigen presenting cell (APC), wherein the cancer antigen is specific to the cancer cells in the subject. Nonlimiting examples of APCs that can be used in the methods of this invention include dendritic cells, macrophages, artificial APCs expressing MHC-I/II/CD80/CD86/OX40L/GITRL, and any combination thereof. The APC can be in the form of whole tumor cell vaccine and/or a peptide vaccine can be administered to the subject. In some embodiments, APCs of this invention can be primed with various kinds of whole cancer cell membranes/extracts as are known in the art.

It will also be understood that an adjuvant can be administered with a peptide vaccine and/or whole tumor vaccine and/or any other cell in the methods of this invention. Furthermore, any of the compositions of this invention can comprise a pharmaceutically acceptable carrier and a suitable adjuvant. As used herein, “suitable adjuvant” describes an adjuvant capable of being combined with the polypeptide and/or fragment and/or nucleic acid of this invention to further enhance an immune response without deleterious effect on the subject or the cell of the subject. A suitable adjuvant can be, but is not limited to, MONTANIDE ISA51 (Seppic, Inc., Fairfield, N.J.), SYNTEX adjuvant formulation 1 (SAF-1), composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Other suitable adjuvants are well known in the art and include QS-21, Freund's adjuvant (complete and incomplete), alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

As set forth above, it is contemplated that in the methods wherein the compositions of this invention are administered to a subject or to a cell of a subject, such methods can further comprise the step of administering a suitable adjuvant to the subject or to a cell of the subject. The adjuvant can be in the composition of this invention or the adjuvant can be in a separate composition comprising the suitable adjuvant and a pharmaceutically acceptable carrier. The adjuvant can be administered prior to, simultaneous with, and/or after administration of the composition containing any of the polypeptides, fragments, nucleic acids and/or vectors of this invention. For example, QS-21, similar to alum, complete Freund's adjuvant, SAF, etc., can be administered within days/weeks/hours (before or after) of administration of the composition of this invention. The effectiveness of an adjuvant can be determined by measuring the immune response directed against the polypeptide and/or fragment of this invention with and without the adjuvant, using standard procedures, as described herein and as are well known in the art.

The compositions of the present invention can also include other medicinal agents, pharmaceutical agents, carriers, diluents, immunostimulatory cytokines, etc.

In some embodiments, the CD4⁺ Th9 cell of this invention can be genetically engineered to produce a chimeric antigen receptor (CAR) that is exposed on the Th9 cell surface, wherein the CAR is specific for cancer cells in the subject. As one nonlimiting example, these cells can be transduced with a viral vector or transfected with a nonviral vector or nucleic acid construct that contains a nucleotide sequence encoding a CAR under conditions whereby the nucleotide sequence is expressed in the Th9 cell and the CAR produced in the cell is transported to the cell surface.

In some embodiments, the CD4⁺ Th9 cell of this invention can be genetically engineered to produce a tumor-specific T cell receptor (TCR) that is exposed on the Th9 cell surface, wherein the TCR is specific for cancer cells in the subject. As one nonlimiting example, these cells can be transduced with a viral vector or transfected with a nonviral vector or nucleic acid construct that contains a nucleotide sequence encoding a TCR under conditions whereby the nucleotide sequence is expressed in the Th9 cell and the TCR produced in the cell is transported to the cell surface.

In some embodiments, the CD4⁺ Th9 cell of this invention can be primed with cancer antigen-loaded APCs to have specificity for cancer cells in the subject.

In some embodiments, the CD4⁺ Th9 cell of this invention can be produced from tumor-infiltrating and/or tumor-draining lymph node T cells.

Nonlimiting examples of a cancer antigen that can be targeted according to methods of this invention include NY-ESO-1, WT-1, MART-1, gp100, gp75, MAGEA3, MAGEA4, HPV16-E6, thyroglobulin, melanoma-associated antigen tyrosinase, CD19, CD22, CD23, CD5, CD30, CD70, CD38, CD138, CD20, CD123, HER2, IL13Ra2, CSPG4, EGFR, EGFRvIII, mesothelin, PSMA (prostate-specific membrane antigen, encoded by the FOLH1 (folate hydrolase 1) gene), CEA (carcinoembryonic antigen), GD2 (disialoganglioside 2), GPC3 (glypican-3), CAIX (carbonic anhydrase IX), L1-CAM (L1 cell adhesion molecule), CA125 (cancer antigen 125, also known as MUC16), CD133 (prominin-1), FAP (fibroblast activation protein), MUC1 (mucin 1), FR-α (folate receptor-α), Lewis-Y, folate receptor β, DKK1, integrin β, other members of the MAGEA family (melanoma antigen family A), including for example, MAGEA1 which comprises members of the larger family of cancer testis (CT) or cancer-germline antigen family, tumor peptides derived from cyclin B1, human cancer antigens targeted by CD4⁺ T cells, GAGE and BAGE antigens, hTERT, PSA, survivin, p53, mutated antigens derived from the protein products of mutated oncogenes such as KRAS, NRAS, and HRAS, new epitopes created by gene translocations and fusions such as BCR-ABL in chronic myelogenous leukemia, ETV6/AML in acute lymphoblastic leukemia, NPM/ALK in anaplastic large-cell lymphomas and ALK in neuroblastomas, cancer neoantigens, including neoantigens that arise in cancer with high mutator phenotype, and any combination thereof. A cancer antigen of this invention can be any cancer antigen now known or later identified, including for examples, antigens listed in the following references: Novellino et al. “A listing of human tumor antigens recognized by T cells: March 2004 update” Cancer Immunology, Immunotherapy 54(3):187-207 (2005); Vigneron et al. “Database of T cell-defined human tumor antigens: the 2013 update” Cancer Immunity 13:15 (2013); Finn. “Human Tumor Antigens Yesterday, Today, and Tomorrow.” Cancer Immunol Res 5(5):347-354 (2107); and the database maintained at cancerresearch.org/scientists/meetings-and-resources/peptide-database, the entire contents of each of which are incorporated by reference herein.

Nonlimiting examples of a cancer of this invention include B cell lymphoma, T cell lymphoma, myeloma, leukemia, hematopoietic neoplasias, thymoma, lymphoma, sarcoma, lung cancer, liver cancer, non-Hodgkins lymphoma, Hodgkins lymphoma, uterine cancer, cervical cancer, endometrial cancer, adenocarcinoma, breast cancer, pancreatic cancer, colon cancer, anal cancer, renal cancer, bladder cancer, prostate cancer, ovarian cancer, primary or metastatic melanoma, squamous cell carcinoma, basal cell carcinoma, brain cancer, angiosarcoma, hemangiosarcoma, head and neck carcinoma, thyroid carcinoma, soft tissue sarcoma, bone sarcoma, testicular cancer, gastrointestinal cancer, stomach cancer, glioblastoma, small cell lung cancer, non-small cell lung cancer and any combination thereof, as well as any other cancer or malignant neoplasm now known or later identified (see, e.g., Rosenberg (1996) Ann. Rev. Med. 47:481-491, the entire contents of which are incorporated by reference herein).

In some embodiments, the methods of this invention can further comprise the steps of administering to the subject one or more chemotherapeutic agents, immunomodulatory agents, ani-inflammatory agents, a surgical procedure and/or radiation, singly or in any combination. Nonlimiting examples of such agents include immune checkpoint blockade agents, such as PD-1, CTLA-4, PD-L1, anti-OX40 agonist monoclonal antibodies (mAbs), anti-GITR agonist mAbs, anti-4-1BB agonist mAbs, etc., as are known in the art. Such agents can be administered to a subject of this invention singly or in any combination and/or ratio, prior to, concurrently with and/or following the administration of the CD4⁺ Th9 cells of this invention.

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the application.

As used herein, “a,” “an” and “the” can mean one or more than one, depending on the context in which it is used. For example, “a” cell can mean one cell or multiple cells.

Also, as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, 10%, ±5%, ±1%, 0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising.”

Also, as used herein, “one or more” means one, two, three, four, five, six, seven, eight, nine, ten, 11, 12, 13, 14, 15, 16, 17, 18, 19, 20, etc.

As used herein, the terms “increase,” “increases,” “increased,” “increasing,” “improve,” “enhance,” and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more.

As used herein, the terms “reduce,” “reduces,” “reduced,” “reduction,” “inhibit,” and similar terms refer to a decrease in the specified parameter of at least about 5%, 10%, 15%, 20%, 25%, 30%, 35%, 40%, 45%, 50%, 55%, 60%, 65%, 70%, 75%, 80%, 85%, 90%, 95%, 97%, or 100%.

As used herein, the term “therapeutically effective amount” or “effective amount” can refer to that amount of a pharmaceutical composition that results in amelioration of symptoms (e.g., reduction in size or elimination of a tumor) and/or a prolongation of survival in a subject. A therapeutically relevant effect relieves to some extent one or more symptoms of a disease or condition or returns to normal either partially or completely one or more physiological or biochemical parameters associated with or causative of the disease or condition.

As used herein, the terms “treating” or “treatment” of a condition or disease can include: (1) inhibiting the disease or condition, i.e., arresting, delaying or reducing the development of the disease or condition and its symptoms; or (2) relieving the disease or condition, i.e., causing regression of the disease or condition and its clinical symptoms. The term “treatment” or “treating,” as used herein, does not encompass 100% cure of cancer. However, in one embodiment, the therapeutic methods described herein can result in 100% reversal of detectable disease.

As used herein, the terms “prophylactic” or “preventative” treatment can include preventing at least one symptom of the disorder, disease or condition, i.e., causing a clinical symptom to not significantly develop in a subject that may develop or be predisposed to the disease but does not yet experience or display symptoms of the disease or condition.

As used herein, the term “subject” can refer to any animal, including, but not limited to, humans and non-human animals (e.g., rodents, arthropods, insects, fish (e.g., zebrafish)), non-human primates, ovines, bovines, ruminants, lagomorphs, porcines, caprines, equines, canines, felines, ayes, etc.), which is to be the recipient of a particular treatment. Typically, the terms “patient” and “subject” are used interchangeably herein in reference to a human subject.

As used herein, “IL-9” refers to a 4-helix bundle cytokine that is produced by T-cells, typically by CD4+ helper cells (e.g., activated Th2 cells, or Th9 cells) but as described herein, also in cytotoxic CD8⁺ Tc9 cells. Alternative names for IL-9 include, but are not limited to, P40, HP40, T-cell growth factor p40, interleukin-9, or P40 cytokine.

As used herein, “adoptive cell transfer” is the process of passively transferring cells, particularly immune-derived cells, into a host with the goal of transferring the immunologic functionality and characteristics into the host. In some embodiments, IL-9 producing cells are used in adoptive cell transfer according to the methods described herein. In some embodiments, Th9 cells are used in adoptive cell transfer according to the methods described herein.

As used herein, the term “peptide” is used to designate a series of residues, typically L-amino acids, connected one to the other typically by peptide bonds between the alpha-amino and carbonyl groups of adjacent amino acids.

An “immunogenic peptide” is a peptide which comprises an allele-specific motif such that the peptide will bind the major histocompatibility complex (MHC) allele and be capable of inducing a cytotoxic T lymphocyte (CTL) response. Thus, immunogenic peptides are capable of binding to an appropriate MHC molecule and inducing a cytotoxic T response against the antigen from which the immunogenic peptide is derived.

As used herein, the term “costimulatory molecule” refers to a molecular component that promotes activation, proliferation and effector function of a T cell after engagement of an antigen specific receptor.

As used herein, the term “cytoplasmic signaling domain” refers to the 5 component of a co-stimulatory molecule or cytokine receptor that exists inside the cell and is responsible for transducing the external signal received to the internal metabolic processes of the cell, thereby altering its phenotype and function.

In embodiments of the present invention, the overexpression of a target cancer antigen by cancer cells allows these cells to be targeted in vitro and in vivo by CAR-expressing primary T cells, wherein the CAR is specific for the target cancer antigen, and in some embodiments, incorporation of endodomains from both CD28 and OX40 molecules mediates costimulation of the T lymphocytes, inducing T cell activation, proliferation, and cytotoxicity against target antigen-positive cancer and/or cancer initiating cells (CICs).

In particular embodiments of the invention, there are methods for killing cancer cells using genetically manipulated T-cells that express a chimeric antigen receptor (CAR) directed against a target cancer antigen. In some embodiments, engagement (antigen binding) of this CAR leads to activation of the linked T-cell receptor C chain and the costimulatory molecules CD28 and OX40.

In particular embodiments of the invention, the CAR receptor comprises a single-chain variable fragment (scFv) that recognizes the target cancer antigen. The skilled artisan recognizes that scFv is a fusion protein of the variable regions of the heavy (VH) and light chains (VL) of immunoglobulins, connected with a short linker peptide of ten to about 25 amino acids. The linker may be rich in glycine for flexibility and/or it may have serine or threonine for solubility, in certain cases. The scFv may be generated by methods known in the art.

In certain aspects, one can use cytokine exodomains or other ligand/receptor molecules as exodomains to provide targeting to the tumor cells.

The skilled artisan recognizes that T cells utilize co-stimulatory signals that are antigen non-specific to become fully activated. In particular cases they are provided by the interaction between co-stimulatory molecules expressed on the membrane of APC and the T cell. In specific embodiments, the one or more costimulatory molecules in the chimeric receptor come from the B7/CD28 family, TNF superfamily, or the signaling lymphocyte activation molecule (SLAM) family. Exemplary costimulatory molecules include one or more of the following in any combination: B7-1/CD80; CD28; B7-2/CD86; CTLA-4; B7-H1/PD-L1; ICOS; B7-H2; PD-1; B7-H3; PD-L2; B7-H4; PDCD6; BTLA; 4-1BB/TNFRSF9/CD137; CD40 Ligand/TNFSF5; 4-1BB Ligand/TNFSF9; GITR/TNFRSF18; BAFF/BLyS/TNFSF13B; GITR Ligand/TNFSF18; BAFF R/TNFRSF13C; HVEM/TNFRSF14; CD27/TNFRSF7; LIGHT/TNFSF14; CD27 Ligand/TNFSF7; OX40/TNFRSF4; CD30/TNFRSF8; OX40 Ligand/TNFSF4; CD30 Ligand/TNFSF8; TAC/TNFRSF13B; CD40/TNFRSF5; 2B4/CD244/SLAMF4; CD84/SLAMF5; BLAME/SLAMF8; CD229/SLAMF3; CD2 CRACC/SLAMF7; CD2F-10/SLAMF9; NTB-A/SLAMF6; CD48/SLAMF2; SLAM/CD150; CD58/LFA-3; CD2; Ikaros; CD53; Integrin alpha 4/CD49d; CD82/Kai-1; Integrin alpha 4 beta 1; CD90/Thy1; Integrin alpha 4 beta 7/LPAM-1; CD96; LAG-3; CD160; LMIR1/CD300A; CRTAM; TCL1A; DAP12; TIM-1/KIM-1/HAVCR; Dectin-1/CLEC7A; TIM-4; DPPIV/CD26; TSLP; EphB6; TSLP R; and HLA-DR.

The CAR of the invention may employ one, two, three, four, or more costimulatory molecules in any combination.

The effector domain is a signaling domain that transduces the event of receptor ligand binding to an intracellular signal that partially activates the T lymphocyte. Absent appropriate co-stimulatory signals, this event is insufficient for useful T cell activation and proliferation. A nonlimiting example of an effector domain of this invention is the effector domain of the T cell receptor zeta chain.

In certain embodiments of the invention, methods of the present invention for clinical aspects are combined with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, and/or increasing the lifespan of a subject with cancer. More generally, these other compositions would be provided in a combined amount effective to kill or inhibit proliferation of the cancer cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir. In the context of the present invention, it is contemplated that cell therapy could be used similarly in conjunction with chemotherapeutic, radiotherapeutic, or immunotherapeutic intervention, in addition to other pro-apoptotic or cell cycle regulating agents.

In some embodiments, the present inventive therapy may precede and/or follow the other agent treatment(s) by intervals ranging from minutes to weeks. In embodiments where the other agent and the therapy of the present invention are applied separately to the subject, one would generally ensure that a significant period of time did not expire between each delivery, such that the agent and inventive therapy would still be able to exert an advantageously combined effect on the cell. In such instances, it is contemplated that one may contact the cell with the multiple modalities within about 12-24 hours of each other and, more preferably, within about 6-12 hours of each other. In some situations, it may be desirable to extend the time period for treatment significantly, however, where several days (2, 3, 4, 5, 6 or 7) to several week(s) (1, 2, 3, 4, 5, 6, 7 or 8) lapse between the respective administrations.

It is expected that the treatment cycles would be repeated as necessary. It also is contemplated that various standard therapies, as well as surgical intervention, may be applied in combination with the inventive cell therapy.

Cancer therapies also include a variety of combination therapies with both chemical and radiation based treatments. Combination chemotherapies include, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

In specific embodiments, chemotherapy for a cancer is employed in conjunction with the methods and compositions of this invention, for example before, during and/or after administration of the methods and compositions invention.

Other agents that cause DNA damage and have been used in cancer treatment include gamma rays, X-rays, and/or the directed delivery of radioisotopes to tumor cells. Other forms of DNA damaging agents are also contemplated such as microwaves and UV-irradiation. It is most likely that all of these agents affect a broad range of damage on DNA, on the precursors of DNA, on the replication and repair of DNA, and/or on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “administered,” “contacted,” “provided to” and “exposed,” when applied to a cell, are used herein to describe the process by which a therapeutic agent is delivered to a target cell and/or is placed in direct juxtaposition with the target cell, e.g., under conditions that facilitate binding of a CAR to a target cancer antigen in and/or on a target cancer cell. In some embodiments, chemotherapy and/or radiation therapy can also be included before, after and/or during the administering, contacting, exposing and/or providing to step to achieve cell killing or stasis. In some embodiments, multiple agents can be delivered to a cell in a combined amount effective to kill the cell or prevent it from dividing.

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and natural killer (NK) cells.

Immunotherapy could thus be used as part of a combined therapy, in conjunction with the present adaptive cell therapy. The general approach for combined therapy is discussed below. Generally, the tumor cell must bear some marker that is amenable to targeting, i.e., is not present on the majority of other cells.

Many tumor markers exist and any of these may be suitable for targeting in the context of the present invention. Nonlimiting examples of common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

Immunotherapy for a cancer of this invention may include interleukin-2 (IL-2) or interferon (IFN), for example.

In other embodiments, the secondary treatment can be a gene therapy in which a therapeutic polynucleotide is administered before, after, and/or at the same time as the present invention methods and compositions. A variety of expression products is encompassed within the invention, including inducers of cellular proliferation, inhibitors of cellular proliferation, and/or regulators of programmed cell death.

Approximately 60% of persons with cancer will undergo surgery of some type, which includes preventative, diagnostic or staging, curative and/or palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the tumor eradicating abilities of the present invention by establishment of an autocrine or paracrine effect on hyperproliferative cells. Increasing intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Nonlimiting examples of suitable chemotherapeutic agents which may be administered with the antibodies or antigen binding fragments as described herein include daunomycin, cisplatin, verapamil, cytosine arabinoside, aminopterin, democolcine, tamoxifen, Actinomycin D, Alkylating agents (including, without limitation, nitrogen mustards, ethylenimine derivatives, alkyl sulfonates, nitrosoureas and triazenes): Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide; Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine, natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as Taxol®), Mithramycin, Deoxyco-formycin, Mitomycin-C, L-Asparaginase, Interferons (especially IFN-a), Etoposide, and Teniposide; Other anti-proliferative cytotoxic agents are navelbene, CPT-11, anastrazole, letrazole, capecitabine, reloxafine, cyclophosphamide, ifosamide, and droloxafine. Additional anti-proliferative cytotoxic agents include, but are not limited to, melphalan, hexamethyl melamine, thiotepa, cytarabin, idatrexate, trimetrexate, dacarbazine, L-asparaginase, camptothecin, topotecan, bicalutamide, flutamide, leuprolide, pyridobenzoindole derivatives, interferons, and interleukins. Preferred classes of antiproliferative cytotoxic agents are the EGFR inhibitors, Her-2 inhibitors, CDK inhibitors, and Herceptin® (trastuzumab). (see, e.g., U.S. Pat. No. 6,537,988; 6,420,377). Such compounds may be given in accordance with techniques currently known for the administration thereof.

As used herein, the term “purified” does not require absolute purity; rather, it is intended as a relative term. Thus, for example, a purified cell population of CD8+Tc9 cells or CD4+ Th9 cells is one in which the percentage of CD8+ Tc9 cells or CD4+ Th9 cells in a population of cells (e.g., in culture) is more pure than CD8+ Tc9 cells or CD4+ Th9 cells in their natural environment, such as within a human subject. In particular examples, substantially purified populations of CD8+ Tc9 cells or CD4+Th9 cells refers to populations of CD8+ Tc9 cells or CD4+ Th9 cells that are at least 50%, 60%, 65%, 70%, 75%, 80%, 90%, 95%, 96%, 97%, 98% or 99% pure. In one embodiment, a substantially purified population of CD8+ Tc9 cells or CD4+ Th9 cells is composed of at least about 70%, such as at least about 80%, such as at least about 90% CD8+ Tc9 cells or CD4+ Th9 cells. That is, the population of CD8+ Tc9 cells or CD4+ Th9 cells includes less than about 20%, such as at least about 10%, of other T lymphocytes such as Tc1 cells. The purity of a CD8+ Tc9 population or CD4+ Th9 cells can be measured based on cell surface characteristics (e.g., as measured by fluorescence activated cell sorting) or by cytokine secretion profile (e.g., as measured by an ELISA assay), as compared to a control.

In some embodiments, prior to administration of the cells of this invention to a subject, the subject's immune system, such as T cells, can be non-selectively or selectively depleted, or ablated, by any method known in the art, for example, selective depletion or ablation of T cells or a specific subset of T cells. Exemplary treatments to induce lymphopenia in a subject prior to cell administration can include but are not limited to the administration of chemotherapeutics and/or total body irradiation.

In one embodiment, the subject's immune system is depleted or ablated by the administration of an induction chemotherapy regimen comprising a therapeutically effective amount of etoposide, doxorubicin, vincristine, cyclophosphamide, and prednisone (EPOCH). In another embodiment, fludarabine can also be administered to improve the depletion of T cells.

Amino acid as used herein refers to a compound having a free carboxyl group and a free unsubstituted amino group on the a carbon, which may be joined by peptide bonds to form a peptide active agent as described herein. Amino acids may be standard or non-standard, natural or synthetic, with examples (and their abbreviations) including but not limited to:
Asp=D=Aspartic Acid
Ala=A=Alanine
Arg=R=Arginine
Asn=N=Asparagine
Cys=C=Cysteine
Gly=G=Glycine
Glu=E=Glutamic Acid
Gln=Q=Glutamine
His=H=Histidine
Ile=I=Isoleucine
Leu=L=Leucine
Lys=K=Lysine
Met=M=Methionine
Phe=F=Phenylalanine
Pro=P=Proline
Ser=S=Serine
Thr=T=Threonine
Trp=W=Tryptophan
Tyr=Y=Tyrosine
Val=V=Valine
Orn=Ornithine
Nal=2-napthylalanine
Nva=Norvaline
Nle=Norleucine
Thi=2-thienylalanine
Pcp=4-chlorophenylalanine
Bth=3-benzothienyalanine
Bip=4,4′-biphenylalanine
Tic=tetrahydroisoquinoline-3-carboxylic acid
Aib=aminoisobutyric acid
Anb=α-aminonormalbutyric acid
Dip=2,2-diphenylalanine
Thz=4-Thiazolylalanine

All peptide sequences mentioned herein are written according to the usual convention whereby the N-terminal amino acid is on the left and the C-terminal amino acid is on the right. A short line (or no line) between two amino acid residues indicates a peptide bond.

“Basic amino acid” refers to any amino acid that is positively charged at a pH of 6.0, including but not limited to R, K, and H.

“Aromatic amino acid” refers to any amino acid that has an aromatic group in the side-chain coupled to the alpha carbon, including but not limited to F, Y, W, and H.

“Hydrophobic amino acid” refers to any amino acid that has a hydrophobic side chain coupled to the alpha carbon, including but not limited to I, L, V, M, F, W and C, most preferably I, L, and V.

“Neutral amino acid” refers to a non-charged amino acid, such as M, F, W, C and A.

“Pharmaceutically acceptable” as used herein means that the compound or composition is suitable for administration to a subject to achieve the treatments described herein, without unduly deleterious side effects in light of the severity of the disease and necessity of the treatment.

“Antibody” or “antibodies” as used herein refers to all types of immunoglobulins, including IgG, IgM, IgA, IgD, and IgE. The term “immunoglobulin” includes the subtypes of these immunoglobulins, such as IgG₁, IgG₂, IgG₃, IgG₄, etc. The antibodies may be of any species of origin, including (for example) mouse, rat, rabbit, horse, or human, or may be chimeric or humanized antibodies. The term “antibody” as used herein includes antibody fragments which retain the capability of binding to a target antigen, for example, Fab, F(ab′)₂, and Fv fragments, and the corresponding fragments obtained from antibodies other than IgG. Such fragments are also produced by known techniques. In some embodiments antibodies may be coupled to or conjugated to a detectable group or therapeutic group in accordance with known techniques.

Furthermore, the term “antibody” as used herein, is intended to refer to immunoglobulin molecules comprising four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDR), interspersed with regions that are more conserved, termed framework regions (FR). In various embodiments of the antibody or antigen binding fragment thereof of the invention, the FRs may be identical to the human germline sequences, or may be naturally or artificially modified. Each VH and VL is composed of three CDRs and four FRs, arranged from amino-terminus to carboxy-terminus in the following order: FR1, CDR1, FR2, CDR2, FR3, CDR3, FR4.

In general, the antibodies and antigen binding fragments thereof of the present invention possess very high affinities, typically possessing K_{D} values of from about 10⁻⁸ through about 10⁻¹ M or higher, for example, at least 10⁻⁸ M, at least 10⁻⁹ M, at least 10⁻¹⁰ M, at least 10⁻¹ M, or at least 10¹² M, when measured by binding to antigen presented on cell surface.

The antibodies and antigen binding fragments thereof of the present invention possess very high affinities, typically possessing EC₅₀ values of from about 10-through about 10⁻¹² M or higher, for example, at least 10⁻⁸ M, at least 10⁻⁹ M, at least 10⁻¹⁰ M, at least 10⁻¹¹ M, or at least 10⁻¹² M, when measured by binding to antigen presented on cell surface.

The term “antigen-binding portion” or “antigen-binding fragment” of an antibody (or simply “antibody portion” or “antibody fragment”), as used herein, refers to one or more fragments, portions or domains of an antibody that retain the ability to specifically bind to an antigen. It has been shown that fragments of a full-length antibody can perform the antigen-binding function of an antibody. Examples of binding fragments encompassed within the term “antigen-binding portion” of an antibody include (i) an Fab fragment, a monovalent fragment consisting of the VL, VH, CL1 and CH1 domains; (ii) an F(ab′)₂ fragment, a bivalent fragment comprising two F(ab)′ fragments linked by a disulfide bridge at the hinge region; (iii) an Fd fragment consisting of the VH and CH1 domains; (iv) an Fv fragment consisting of the VL and VH domains of a single arm of an antibody; (v) a dAb fragment (Ward et al. (1989) Nature 241:544-546), which consists of a VH domain; and (vi) an isolated complementary determining region (CDR). Furthermore, although the two domains of the Fv fragment, VL and VH, are coded for by separate genes, they can be joined, using recombinant methods, by a synthetic linker that enables them to be made as a single contiguous chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies, are also encompassed (see e.g., Holliger et al. (1993) Proc. Natl. Acad Sci. USA 90:6444-6448).

The term “epitope” refers to an antigenic determinant that interacts with a specific antigen binding site in the variable region of an antibody molecule known as a paratope. A single antigen may have more than one epitope. Epitopes may be either conformational or linear. A conformational epitope is produced by spatially juxtaposed amino acids from different segments of one (or more) linear polypeptide chain(s). A linear epitope is an epitope produced by adjacent amino acid residues in a polypeptide chain. In certain embodiments, an epitope may include other moieties, such as saccharides, phosphoryl groups, or sulfonyl groups on the antigen.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-1soleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403 410 and Altschul et al. (1997) Nucleic Acids Res. 25:3389 402, each of which is herein incorporated by reference in its entirety.

“Therapeutic group” means any suitable therapeutic group, including but not limited to radionuclides, chemotherapeutic agents and cytotoxic agents.

“Radionuclide” as described herein may be any radionuclide suitable for delivering a therapeutic dosage of radiation to a tumor or cancer cell, including but not limited to ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸Au, ¹⁶⁶Ho, ^{113m}In, ^{115m}In, ¹²³I, ¹²⁵I, ¹³¹I, ¹⁸⁹Ir, ¹⁹¹Ir, ¹⁹²Ir, ¹⁹⁴Ir, ⁵²Fe, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Se, ⁴⁷Se, ⁷²Se, ⁷⁵Se, ¹⁰⁵Ag, ⁸⁹Sr, ³⁵S, ¹⁷⁷Ta, ¹¹⁷mSn, ¹²¹Sn, ¹⁶⁶Yb, ¹⁶⁹Yb, ⁹⁰Y, ²¹²Bi, ¹¹⁹Sb, ¹⁹⁷Hg, ⁹⁷Ru, ¹⁰⁰Pd, ^{101m}Rh, and ²¹²Pb.

“Cytotoxic agent” as used herein includes but is not limited to ricin (or more particularly the ricin A chain), aclacinomycin, diphtheria toxin. Monensin, Verrucarin A, Abrin, Vinca alkaloids, Tricothecenes, and Pseudomonas exotoxin A.

“Detectable group” as used herein includes any suitable detectable group, such as radiolabels (e.g. ³⁵S, ¹²⁵I, ¹³¹I, etc.), enzyme labels (e.g., horseradish peroxidase, alkaline phosphatase, etc.), fluorescence labels (e.g., fluorescein, green fluorescent protein, etc.), etc., as are well known in the art and used in accordance with known techniques.

The active agents described above (e.g., a Th9 cell) may be formulated for administration in a pharmaceutical carrier in accordance with known techniques. See, e.g., Remington, The Science And Practice of Pharmacy (latest edition). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the subject. The carrier may be a liquid and is preferably formulated with the compound as a unit-dose formulation which may contain from 0.01 or 0.5% to 95% or 99% by weight of the active compound. The carrier may be sterile or otherwise free from contaminants that would be undesirable to administer or deliver to a subject.

In addition, populations of the cells of this invention can be cryopreserved and thawed prior to administration to a subject.

Formulations of the present invention suitable for parenteral administration comprise sterile aqueous and non-aqueous injection solutions of the active compound, which preparations are preferably isotonic with the blood of the intended subject. These preparations may contain anti-oxidants, buffers, bacteriostats and solutes which render the formulation isotonic with the blood of the intended subject.

The active agents may be administered by any medically appropriate procedure, including but not limited to, intravenous, intratumor, intraperitoneal and/or intra-arterial administration.

Active agents may be provided in lyophylized form in a sterile aseptic container or may be provided in a pharmaceutical formulation in combination with a pharmaceutically acceptable carrier, such as sterile pyrogen-free water or sterile pyrogen-free physiological saline solution.

Dosage of the agents and compositions of this invention for the methods of use described herein will depend, among other things, on the condition of the subject, the particular disorder being treated, the route of administration, the nature of the therapeutic agent employed, and the sensitivity of the subject to the particular agent(s).

In some embodiments, the Th9 cells can be in a volume of a liter or less, can be 500 ml or less, 250 ml or 100 ml or less. Hence the density or dose of the desired cells can be from about 1×10⁶ cells to about 1×10¹² cells, and in some embodiments can be from about 1×10⁸ cells to about 1×10¹¹ cells. In some embodiments, Th9 cells in these amounts can be utilized for the treatment of cancer in adult humans, compared to about 5×10⁶-5×10⁷ cells used in mice.

As a nonlimiting example, one or more than one dose of a lymphopenia-inducing agent and/or treatment can be administered to a subject, followed by the administration of one or more than one dose of Th9 cells. The subject can additionally receive Tc9/Tc1 cells and/or one or more than one antigen presenting cell (APC) that has been primed to have specificity for the cancer in the subject being treated. In some embodiments, the Th9 and/or Tc9/Tc1 cells can be CAR T cells that produce a chimeric antigen receptor on the T cell surface that has specificity for the tumor cells being targeted in the subject. In such embodiments involving the administration of CAR T cells to the subject, an APC may or may not be administered to the subject.

Nonlimiting examples of how the Th9 cells of this invention can be primed are as follows: Peripheral blood mononuclear cells (PBMCs), naïve T cells, unselected T cells and/or tumor-infiltrating T cells are contacted with an immunogenic peptide and/or loaded APCs, coated or soluble anti-CD3/anti-CD28 mAbs, or anti-CD3/anti-CD28 conjugated beads to prime the T cells. T cells are also primed in the presence of any Th9 polarization conditions. Examples of polarization conditions include one or more of the following agents in any combination: IL-2, IL-4, TGF-β family cytokines, IL-1β, GITRL, OX40L, anti-GITR agonist mAbs, anti-OX40 agonist mAbs, TNF-α, IL-6, IL-7, IL-15 and/or anti-IFN-γ monoclonal antibodies.

In the treatment of cancers or tumors, the agents and compositions of the present invention may optionally be administered in conjunction with other, different, cytotoxic agents such as chemotherapeutic or antineoplastic compounds or radiation therapy useful in the treatment of the disorders or conditions described herein (e.g., chemotherapeutics or antineoplastic compounds). The other compounds may be administered prior to, concurrently and/or after administration of the antibodies or antigen binding fragments thereof of this invention. As used herein, the word “concurrently” means sufficiently close in time to produce a combined effect (that is, concurrently may be simultaneously, or it may be two or more administrations occurring before or after each other)

As used herein, the phrase “radiation therapy” includes, but is not limited to, x-rays or gamma rays which are delivered from either an externally applied source such as a beam or by implantation of small radioactive sources.

Nonlimiting examples of suitable chemotherapeutic agents which may be administered with the agents and compositions as described herein include daunomycin, cisplatin, verapamil, cytosine arabinoside, aminopterin, democolcine, tamoxifen, Actinomycin D, Alkylating agents (including, without limitation, nitrogen mustards, ethylenimine derivatives, alkyl sulfonates, nitrosoureas and triazenes): Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide; Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine, Natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as Taxol), Mithramycin, Deoxyco-formycin, Mitomycin-C, L-Asparaginase, Interferons (especially IFN-α), Etoposide, and Teniposide; Other anti-proliferative cytotoxic agents are navelbene, CPT-11, anastrazole, letrazole, capecitabine, reloxafine, cyclophosphamide, ifosamide, and droloxafine. Additional anti-proliferative cytotoxic agents include, but are not limited to, melphalan, hexamethyl melamine, thiotepa, cytarabin, idatrexate, trimetrexate, dacarbazine, L-asparaginase, camptothecin, topotecan, bicalutamide, flutamide, leuprolide, pyridobenzoindole derivatives, interferons, and interleukins. Preferred classes of antiproliferative cytotoxic agents are the EGFR inhibitors, Her-2 inhibitors, CDK inhibitors, and Herceptin® (trastuzumab). (see, e.g., U.S. Pat. No. 6,537,988; 6,420,377). Such compounds may be given in accordance with techniques currently known for the administration thereof.

The invention further provides polynucleotides comprising a nucleotide sequence encoding a chimeric antigen receptor of the invention as described above. The polynucleotides may be obtained, and the nucleotide sequence of the polynucleotides determined, by any method known in the art. For example, if the nucleotide sequence of the components of the chimeric antigen receptor are known, a polynucleotide encoding the components may be assembled from chemically synthesized oligonucleotides, which involves the synthesis of overlapping oligonucleotides containing portions of the sequence encoding the components of the chimeric antigen receptor, annealing and ligation of those oligonucleotides, and then amplification of the ligated oligonucleotides by PCR. Alternatively, a polynucleotide encoding a chimeric antigen receptor may be generated from nucleic acid from a suitable source. Amplified nucleic acids generated by PCR may then be cloned into replicable cloning vectors using any method well known in the art.

The present invention is explained in greater detail in the following non-limiting examples.

### EXAMPLES

The following examples provide illustrative embodiments. Certain aspects of the following examples are disclosed in terms of techniques and procedures found or contemplated by the present inventors to work well in the practice of the embodiments. In light of the present disclosure and the general level of skill in the art, those of skill will appreciate that the following example are intended to be exemplary only and that numerous changes, modifications, and alterations can be employed without departing from the scope of the presently claimed subject matter. These examples should in no way be construed as limiting the broad scope of the invention.

### Example 1

Mice. C57BL/6 (B6), Cd8a^{-/-} (B6.129S2-Cd8a^{tm1Mak}/J), Ifng⁺ (B6.129S7-Ifng^{tm1Ts}/J), Eomes^{fl/fl} (B6.129S1(Cg)-Eomes^{tm1.1Bflu}/J), Stat6^{-/-} (B6.129S2(C)-Stat6^{tm1Gru}/J), Cd4-Cre (B6.Cg-Tg(Cd4-cre)1Cwi/BfluJ), OT-II (C57BL/6-Tg(TcraTcrb)425Cbn/J), CD45.1 (B6.SJL-Ptprca Pepcb/BoyJ), and TRP-1 (B6.Cg-Rag1^{tm1Mom} Tyrp1^{B-w}Tg(Tcra,Tcrb)9Rest/J) mice were purchased from Jackson Laboratory. Traf6^{fl/fl} and Il9r^{-/-} mice on the B6 background were generated as described previously. Il9^{-/-} mice on the B6 background were provided by Dr. Dong Chen from Tsinghua University. CD45.1-OT-II, Ifng^{-/-}-CD45.1-OT-II, Il9^{-/-}-CD45.1-OT-II, Eomes^{fl/fl}-Cd4-Cre-OT-II, and Traf6^{fl/fl}-Cd4-Cre-OT-II mice were generated by crossing and backcrossing the existing mice above. Male and female 6- to 8-week-old mice were used for each animal experiment. The studies were approved by the Institutional Animal Care and Use Committees of the Cleveland Clinic Foundation and the Wake Forest School of Medicine.

Cell lines. Wild-type B16 and B16 melanoma cell lines (ATCC) were transfected with OVA (B16-OVA) and cultured in Iscove's Modified Dulbecco's Medium (Invitrogen) supplemented with 10% heat-inactivated fetal bovine serum (Thermo Scientific), 100 U/ml penicillin-streptomycin, and 2 mM L-glutamine (both from Invitrogen).

In vitro Th cell differentiation. Naïve CD4⁺CD62L⁺ T cells were purified from spleens of OT-II or TRP-1 mice and differentiated into Th1, Th9, or Th17 cells according to established methods. OVA- or TRP-1-specific naïve CD4⁺ T cells were cultured for 3 days with irradiated splenic APCs from C57BL/6 mice in the presence of OVA₃₂₃₋₃₃₉ peptide or TRP-1₁₀₆₋₁₃₃ (5 μg/ml) with:
(a) Th9-polarized medium supplemented with IL-4 (10 ng/ml), TGF-β1 (1 ng/ml), and anti-IFN-γ monoclonal antibodies (mAbs; 10 μg/ml);
(b) Th1-polarized medium supplemented with IL-2 (30 ng/ml), IL-12 (4 ng/ml), and anti-IL-4 mAbs (10 μg/ml);
(c) Th17-polarized medium supplemented with IL-6 (30 ng/ml), TGF-β1 (2.5 ng/ml), and anti-IFN-γ mAbs (10 μg/ml);
(d) Th2-polarized medium supplemented with IL-4 (10 ng/ml) and anti-IFN-γ mAbs (10 μg/ml);
(e) pTh17-polarized medium supplemented with IL-6 (30 ng/ml), IL-1β (20 ng/ml), IL-23 (50 ng/ml), and anti-IFN-γ mAbs (10 g/ml);
(f) Th17 (αIL-2+IL-23)-polarized medium supplemented with IL-6 (30 ng/ml), IL-1β (20 ng/ml), TGF-β1 (2.5 ng/ml), IL-21 (100 ng/ml), anti-IL-4 mAbs (10 μg/ml), anti-IL-2 mAbs (10 g/ml) and anti-IFN-γ mAbs (10 μg/ml);
(g) pTh17 (low TGFβ)-polarized medium supplemented with IL-6 (30 ng/ml), IL-1β (20 ng/ml), IL-23 (50 ng/ml), TGF-β1 (0.25 ng/ml) and anti-IFN-γ mAbs (10 μg/ml).
   After the initial 3-day culture, cells were provided with IL-2 (5 ng/ml), except Th17 (αIL-2+IL-23) cells which received IL-2 (5 ng/ml) plus IL-23 (50 ng/ml). After culture for a total of 5 days, differentiated Th cells were depleted of dead cells and used in animal studies. In some experiments, cells were restimulated for 5 hours with OVA-peptide in presence of a protein transport inhibitor (GolgiPlug, BD Biosciences) before ICS using a Cytofix/Cytoperm kit (BD Biosciences). In some experiments, naïve CD4⁺CD62L⁺ T cells may be activated as indicated in the polarized condition with plate-bound anti-CD3 mAbs (2 μg/ml, clone 17A2, eBioscience) and soluble 30 anti-CD28 mAbs (1 μg/ml, clone 37.51, eBioscience).

Viral production and transduction. Viruses were packaged in 293T cells transfected with Lipofectamine 2000 (Life Science). Viral supernatant was harvested from day 1 to day 3, filtered with a 0.45-mm filter, concentrated with PEG-itVirus Precipitation Solution, and stored at −80° C. until use. For the transfection, naïve CD4⁺CD62L⁺ T cells were activated in the polarized condition for 24 hours and then were mixed with the virus and 10 μg/ml protamine sulfate (Sigma), followed by centrifugation for 120 min at 1,800 rpm at 32° C. GFP⁺ T cells were sorted for some experiments.

Real-time PCR. Total RNA was extracted from T cells using the RNeasy Mini kit (Qiagen) according to the manufacturer's instructions. Genes were expressed with specific primers and analyzed by using SYBR green real-time PCR (Applied Biosystems). Expression was normalized to the expression of the housekeeping gene Gapdh.

Tumor models and adoptive transfer. Mice received subcutaneous (s.c.) abdominal injection with 1×10⁶ B16 or B16-OVA tumor cells. At 10 days after tumor injection, mice (5/group) were treated with adoptive transfer of 2.5×10⁶ Th1, Th9, or Th17 cells, followed by intravenous (i.v.) injection of 2.5×10⁵ peptide-pulsed bone marrow-derived dendritic cells generated as previously described. Cyclophosphamide (CTX, Sigma) was administrated intraperitoneally (i.p.) as a single dose at 200 mg/kg 1 day before T-cell transfer. Mice were sacrificed at indicated days, and tumor-draining lymph nodes and splenocytes were analyzed. The number of transferred cells in spleens was calculated by multiplying the total number of viable splenocytes by the percentages of transferred populations. In some experiments, transferred T cells were sorted from splenocytes for indicated analyses.

Flow cytometry and western blot analysis. FITC-, PB-, APC- or PerCP-conjugated mAbs (1:100 dilution) were used for staining after Fc blocking, and analyzed using a FACS Fortessa flow cytometer or MACSQuant. Ki67 staining was performed using a Foxp3 staining kit with anti-Ki67 mAbs.

For Western blot, mAbs from Santa Cruz Biotechnology were used at a 1:500 dilution. mAbs from Cell Signaling and used at a 1:1000 dilution. For some experiments, we prepared cytoplasmic and nuclear extracts from cells using the NE-PER Nuclear and Cytoplasmic Extraction kit.

CFSE labeling and cytotoxicity assay. In some experiments, Th cells were incubated for 5 minutes at 37° C. with 1 μM CFSE in PBS, and then washed extensively. We measured proliferation of T cells by the relative CFSE dilution method after stimulation or transfer into tumor-bearing mice. In the cytotoxicity assay, B16-OVA target cells or B16 non-target cells for OT-II T cells were labeled with 5 μM CFSE. B16-OVA target cells or B16 non-target control cells were incubated alone in triplicate with the OT-II T cells at a 1:10 effector-to-target ratio. For TRP-1 T cells, B16 target cells or MC38 non-target control cells were used. After 18 hours, CFSE⁺ tumor cells from each target and control well were stained using FVD and analyzed by FACS. FVD⁺ tumor cells were considered as dead cells. The percent specific lysis was calculated as (FVD⁺ target−FVD⁺ control)×100%.

Chromatin immunoprecipitation. ChIP assay was performed with a ChIP assay kit (Millipore) according to the manufacturer's instructions. Chromatin was extracted from OT-II-Th1, Th2, Th9, and Th17 cells differentiated for 3 days and fixed with formaldehyde. For the chromatin immunoprecipitation, anti-Pu.1 (sc-390659) and anti-Stat6 (sc-981X) were purchased from Santa Cruz Biotechnology and used at a 1:20 dilution and isotype-matched control antibodies were from Cell Signaling and used at a 1:20 dilution. As the predicted Stat6 binding site is adjacent to the Pu.1 binding site, the precipitated DNA was analyzed by RT-PCR with the following two primer sets surrounding the Pu.1 binding site at the Traf6 promoter region:

(SEQ ID NO: 1)
5′-CTCTCCCGTGACAATGTTGGA-3′
and
(SEQ ID NO: 2)
5′-CTCCACGCTGAAGCCTTACC-3′
(SEQ ID NO: 3)
5′-TGTTGGAGAATGGGATCATGC-3′
and
(SEQ ID NO: 4)
5′-CTCGCTAGGAGCAGCAAGG-3′

To evaluate chromatin modification status, tri-acetyl-histone H3 (K27), mono-methyl-histone H3 (K4), tri-methyl-histone H3 (K4), tri-methyl-histone H3 (K27) mAbs (all from Cell Signaling, 1:20 dilution) were used for the chromatin immunoprecipitation. The precipitated DNA was analyzed by RT-PCR with the following primer sets in the region of mouse Traf6 promoter:

(SEQ ID NO: 5)
5′-GGAGGGGACAGCTATACGCA-3′
and
(SEQ ID NO: 6)
5′-TGTGTGCTCATCACGCAGTT-3′
(SEQ ID NO: 7)
5′-AGCTCTCCCGTGACAATGTT-′3
and
(SEQ ID NO: 8)
5′-TTCCTCGGACCAGTGCAAAA-′3
(SEQ ID NO: 9)
5′-TCTACTTACCTTACCTAACAGCCT-′3
and
(SEQ ID NO: 10)
5′-GCACAATGCAATAGATGCCCA-3′;

the following primer sets in the region of mouse Traf6 enhancer:

(SEQ ID NO: 11)
5′- AAGGGACTCACCAAGAACCT-3′
and
(SEQ ID NO: 12)
5′-GCTCCAAATACAAGAGCAGCC-3′
(SEQ ID NO: 13)
5′-TACTGACTGCTGTGTTAGCTGGAA-′3
and
(SEQ ID NO: 14)
5′-GCAGAGATGCACTGTTCCCT-′3
(SEQ ID NO: 15)
5′- TGGACAGGGGCACTAAGACT-′3
and
(SEQ ID NO: 16)
5′-GAGCTCTGGGCTGTCTCTTC-3′

Values were subtracted from the amount of IgG control and were normalized to the corresponding input control.

Luciferase reporter assays. Using the University of California Santa Cruz Genome Browser, we identified and analyzed the genetic sequence 1 Kb upstream of the mouse Traf6 promoter. Potential transcription factor binding sites were predicted using the following online bioinformatics tools: TRANSFAC, Patch, and GPMiner. High confidence binding sites (87.5% likelihood cutoff) were accepted for additional analysis. Using these 3 tools, we manually identified 19 transcription factors as shown in Table 2.

HEK 293T cells were transiently transfected with a 1256-bp mouse luciferase reporter vector pEZX-PG04 (mTraf6-PG04) inserted into the Traf6 promoter (Genecopoeia) or control vector (NEG-PG04) along with expression vectors for Stat6, Stat5, Stat3, Pu.1, and NF-κB molecules (p50, p52, RelA, RelB and c-Rel, Addgene) by Lipofectamine 2000 (Invitrogen). Promoter activity was measured with the Secrete-Pair Dual Luminescence Assay Kit (GeneCopoeia) according to the manufacturer's instructions. Values are expressed as the mean±S.D. of relative luciferase units normalized to the internal control.

Microarray analysis. Total RNA was extracted with the RNeasy Mini kit (Qiagen) from CD45.1⁺CD4⁺ Th cells sorted from spleens of tumor-bearing mice 12 days after transfer. RNA samples were sent to the Cleveland Clinic Genomics Core for quality evaluation using an Agilent Bioanalyzer. Samples with intact 18S and 28S ribosomal RNA bands with RIN>8.5 were processed for microarray analysis performed with a Mouse Ref-8 v2.0 Expression BeadChip Kit in the Cleveland Clinic 30 Genomics Core. The microarray data were deposited in the NCBI Gene Expression Omnibus (GEO) database under accession number GSE97087. GSEA was run for each cell subset in pre-ranked list mode with 1000 permutations (nominal p-value cutoff <0.01). The early memory signature gene set was selected from an existing publication of genes differentially expressed by >2 fold in primary versus quaternary cells. The mature effector gene set was selected from the same study of genes differentially expressed by >2 fold in quaternary versus primary cells. The T cell exhaustion-associated signature gene sets (down and up) from the Broad Institute Molecular Signature Database were used:

### (GSE24081_CONTROLLER_VS_PROGRESSOR_HIV_SPECIFIC_CD8_TCELL_DN) and

### (GSE24081_CONTROLLER_VS_PROGRESSOR_HIV_SPECIFIC_CD8_TCELL_UP).

Statistical analyses. For statistical analysis, Student's t-test was used. A P value less than 0.05 was considered statistically significant. Results are presented as mean±s.d. unless otherwise indicated.

Table 1 provides Reagents or Resources.

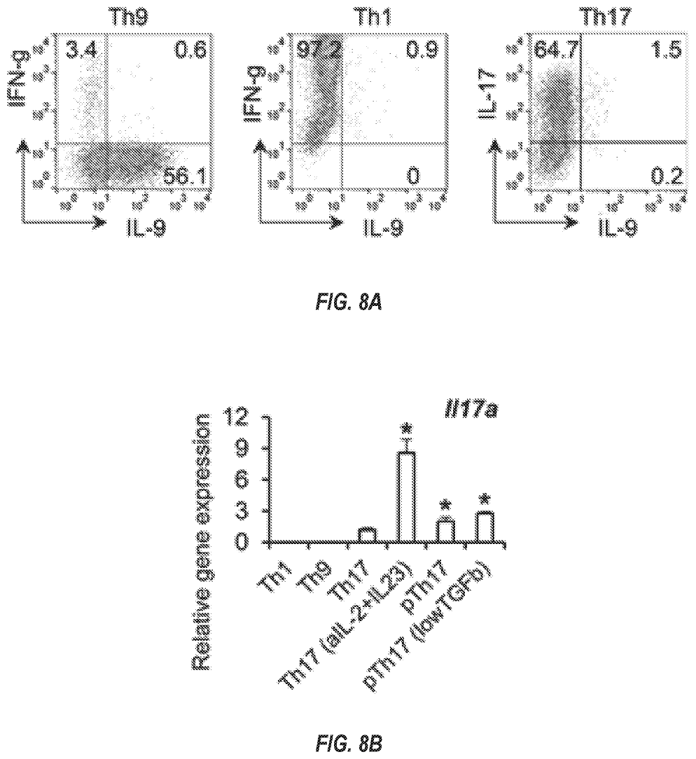

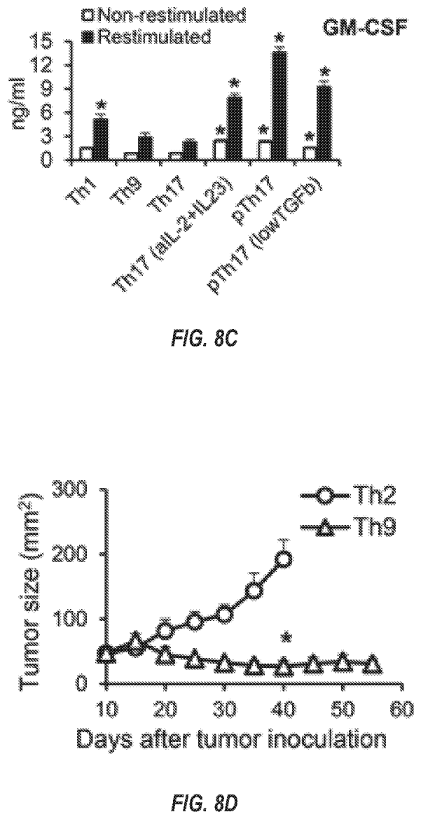

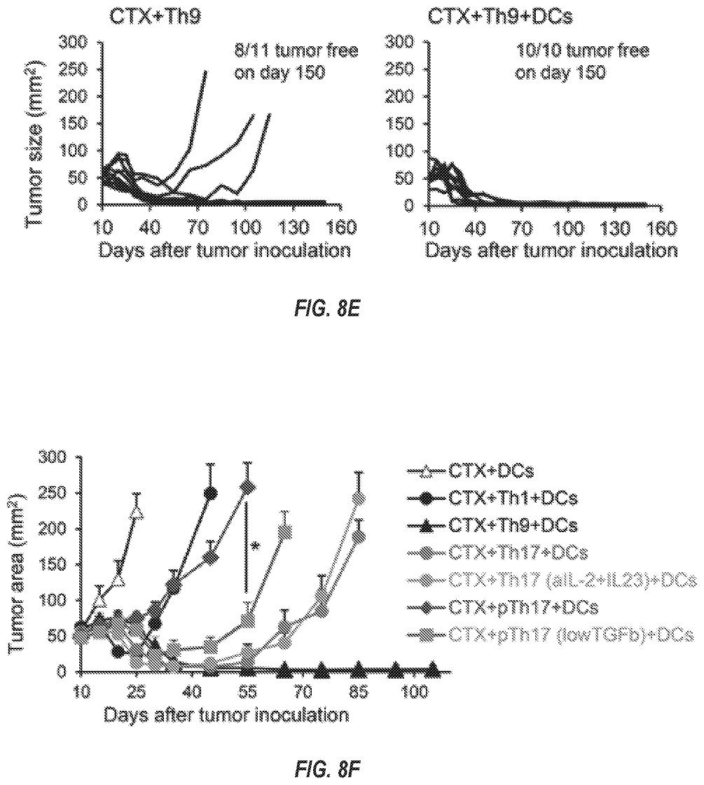

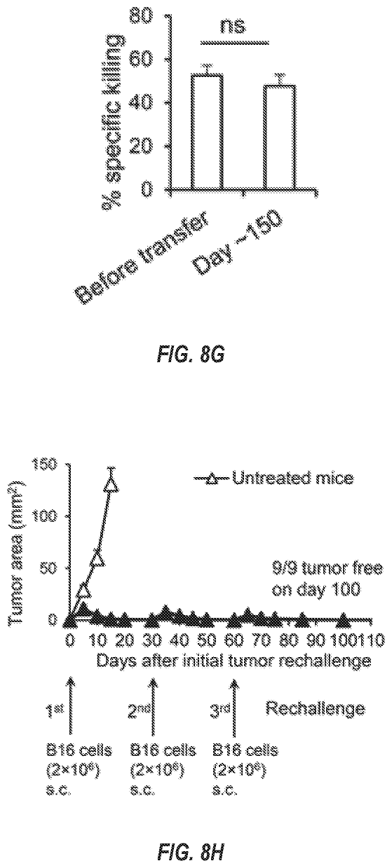

Transfer of Th9 cells eradicates advanced late-stage tumor and leads to long-term survival. Tumor-specific Th9 cells were generated by priming OT-II or TRP-1 naïve CD4⁺CD62L⁺ T cells with peptide-loaded antigen-presenting cells (APCs; irradiated, T cell-depleted splenocytes) for 5 days in Th9-polarized medium. As FIGS. 8A-8C show, differentiated Th9 cells typically were more than 55% IL-9-expressing CD4⁺ T cells, with limited production of IFN-γ, IL-4 or IL-17. In addition, we generated (cultured 5 days) Th1 cells as a control because cytotoxic Th1 cells are therapeutically useful CD4⁺ T cells for ACT in the clinic. We also generated (cultured 5 days) Th17 cells as an additional control because these cells represent the T-cell lineage that may possess the highest antitumor efficacy among CD4⁺ T cell subsets tested so far.

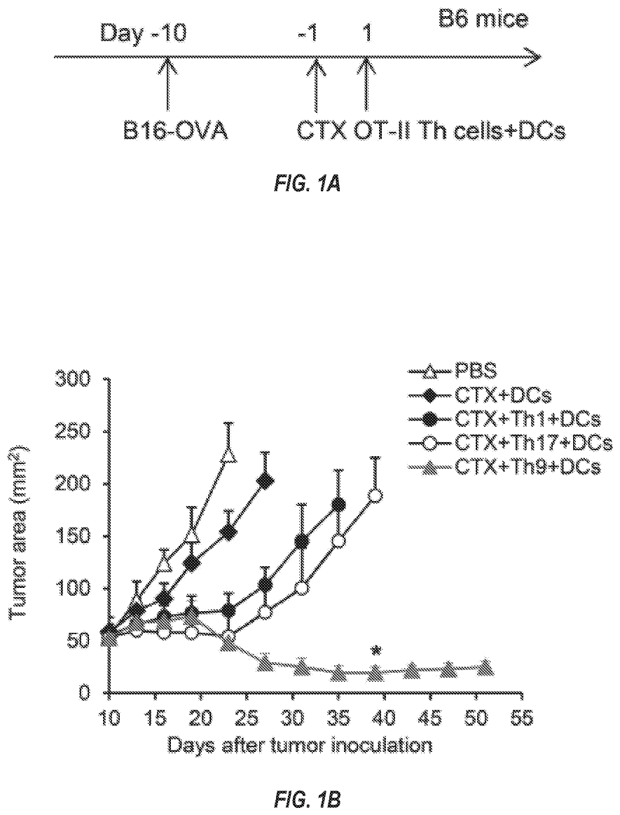

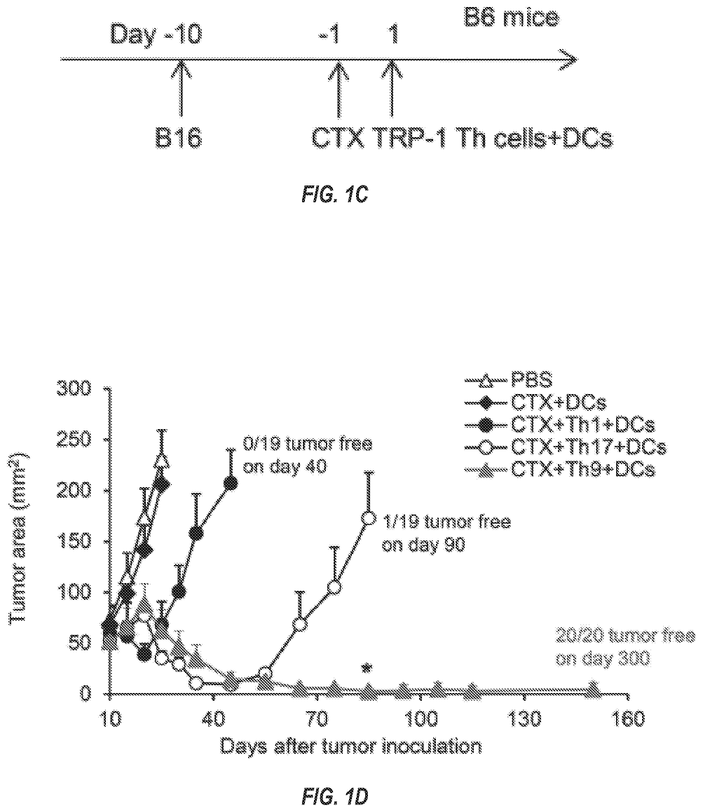

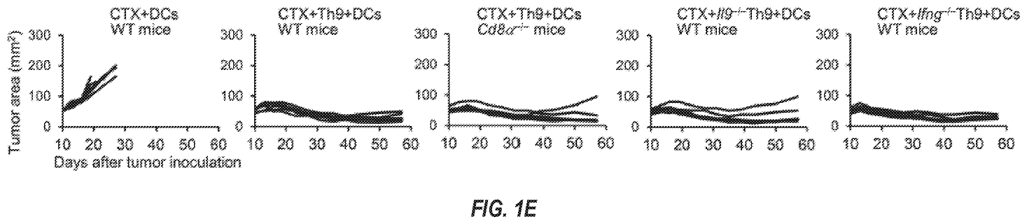

To test our central hypothesis that Th9 cells can be utilized as a potential CD4⁺ T-cell subset for ACT of cancer, we performed studies by transferring OVA-specific CD45.1⁺ OT-II Th1, Th17, or Th9 cells into CD45.2⁺ WT C57BL/6 (B6) mice bearing large (˜8×7 mm), established B16-OVA melanoma (FIG. 1A). One day before T-cell transfer the tumor-bearing mice were given one dose of cyclophosphamide (CTX, 200 mg/kg) to induce temporary lymphopenia, which is frequently induced as part of clinical ACT protocols to promote homeostatic proliferation of transferred T cells. Mice also received adjuvant OVA peptide-pulsed DC vaccination on the day of transfer, which is frequently used to boost the antitumor responses during ACT. Surprisingly, only Th9 cells mediated significant tumor regression that resulted in long-term survival, whereas Th1, Th17 and Th2 cell treatment induced only temporary tumor regression, which was followed by aggressive recurrence (FIG. 1B and FIG. 8D).

Because OT-II cells target the OVA antigen, which is not normally expressed by melanoma tumor cells, we next used the tyrosinase-related protein (TRP)-1 model of adoptive immunotherapy, which reproduces the clinical challenge of targeting gp75 tumor/self-antigen in the poorly immunogenic B16 melanoma. CD45.2⁺ TRP-1-Th1, Th17, or Th9 cells were transferred into CD45.1⁺ B6 mice bearing large established B16 melanomas (˜8×7 mm) in conjunction with CTX administration and DC vaccination (FIG. 1C). Similar to previous reports, Th17 cells more potently induced tumor rejection compared to Th1 cells (FIG. 1D). However, surprisingly, only the Th9 cell transfer eradicated these advanced late-stage tumors long-term, with all treated mice remaining tumor-free at 300 days, whereas Th1 and Th17 cell-treated mice suffered relapse by 3 weeks and 8 weeks, respectively (FIG. 1D). DC vaccination seemed to be required for optimal antitumor responses of Th9 cells (FIG. 8E). In addition, Th9 cells also exerted stronger antitumor activity compared to pathogenic Th17 (pTh17) cells or other types of “Th17” cells generated by different polarizing conditions (FIG. 8F). The Th9 cells, but not the Th17 cells, protected the mice against 3 sequential rechallenges with B16 tumor cells starting at 150 days after the Th cell transfer (FIGS. 8G-8H).

We have reported that in tumor prevention models with low tumor burden, Th9 cells promote CD8⁺ CTL-mediated antitumor immune response. However, in a more clinical scenario (e.g., late-stage advanced tumor burden and lymphodepleting conditions during ACT), the relative contributions of transferred Th9 cells versus induced host CD8⁺ CTLs in eradicating large tumors has not been explored. In this study, although we also observed that Th9 cells induced a significant increase in tumor-infiltrating tumor (OVA)-specific CD8⁺ T cells (FIG. 8I), deficiency in host CD8⁺ T cells only slightly affected the antitumor efficacy of Th9 cell transfer as compared with that in WT mice under lymphopenic conditions (FIG. 1E). This indicates that tumor-specific Th9 cells may be the major effector responsible for eradicating tumor cells in vivo. In addition, we found unexpectedly that IL-9 deficiency in Th9 cells also only marginally affected their efficacy (FIG. 1E). Finally, our results suggested that Th9 cells were not a significant IFN-γ producer, nor did they require IFN-γ to exert their antitumor function because Ifng^{-/-}Th9 cells exerted intact antitumor immunity (FIG. 1E and FIG. 8J). Taken together, these results suggest that tumor-specific Th9 cells might be an ideal T cell subset for ACT, whereas IL-9 production and the induced CD8⁺ CTL responses can be included for their optimal antitumor function.

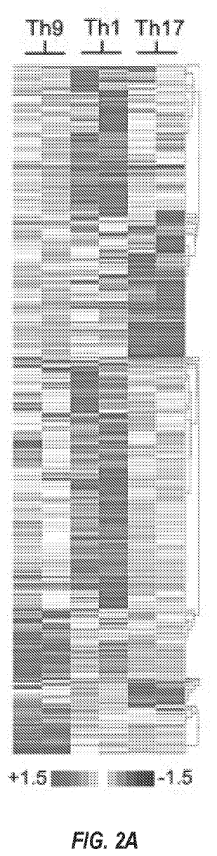

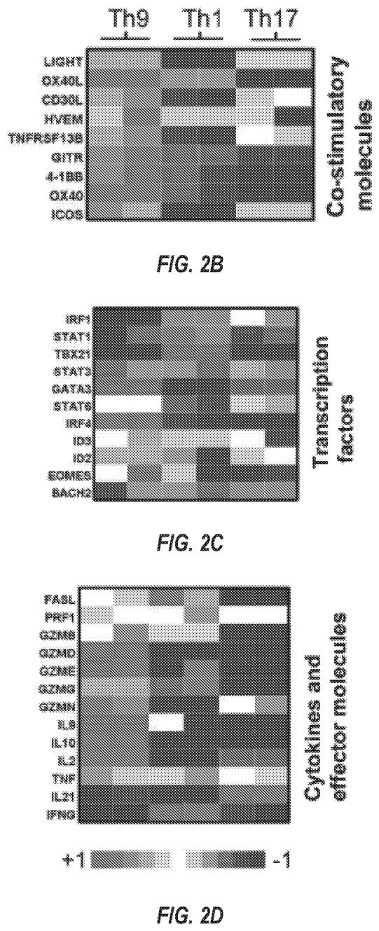

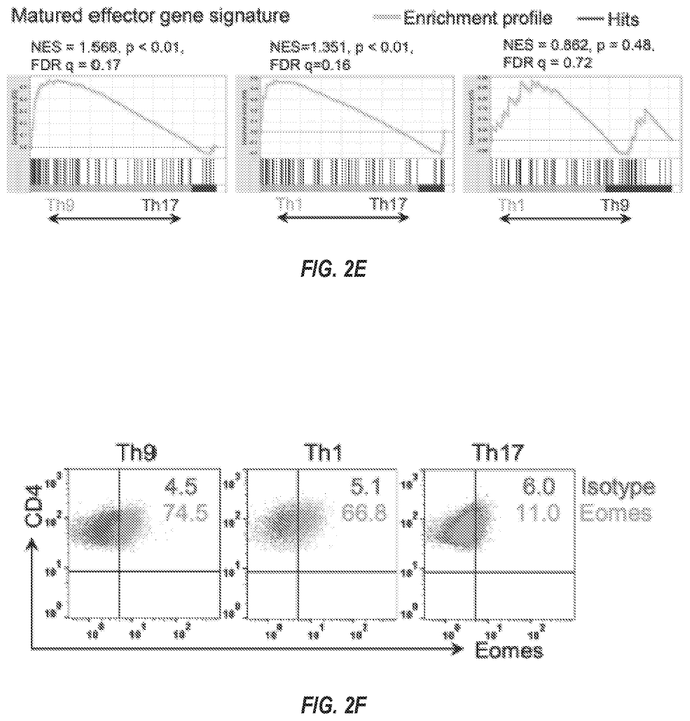

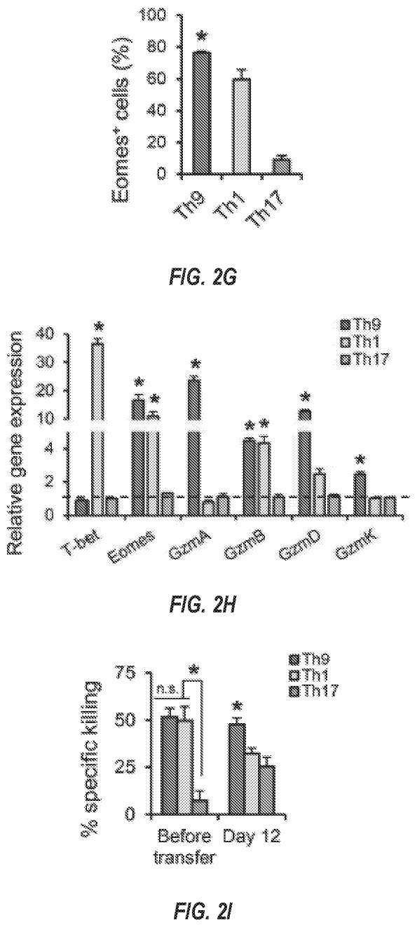

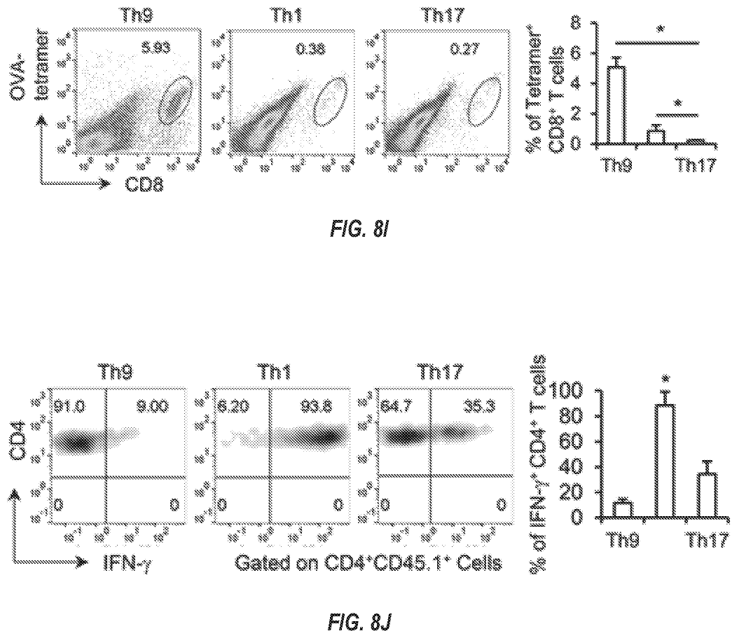

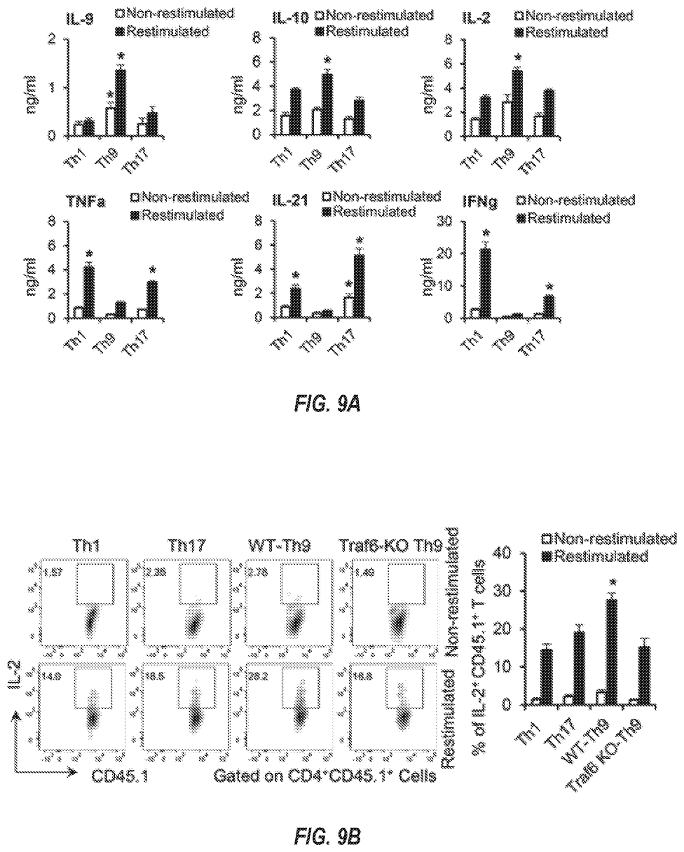

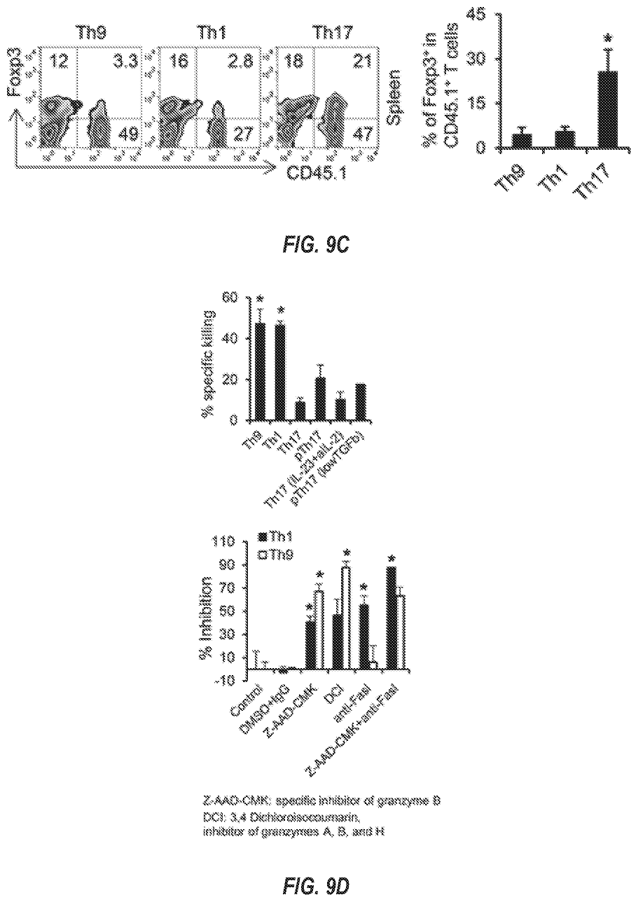

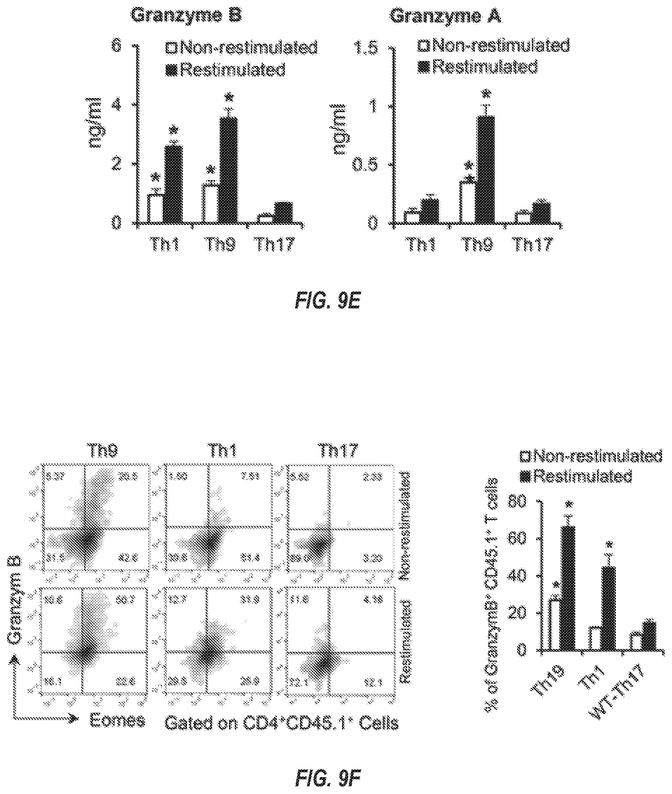

Th9 cells are distinct mature effector T cells. Current advances suggest that T cells exhibiting the long-lived early memory and/or stem cell-like features (Th17 paradigm) should be selected for ACT. Th17 cells are endowed with an enhanced capacity to survive/self-renew, generate Th1-like effector progeny, and enter the memory pool with an anti-tumor efficacy superior to that of short-lived terminally differentiated Th1 cells (Th1 paradigm) for cancer therapy. However, the phenotype of Th9 cells beyond IL-9 production has yet to be studied sufficiently, and the extraordinary ability of Th9 cells to completely cure large advanced tumors prompted us to explore their T cell features. We sorted CD45.1⁺ OVA-specific Th9-, Th17-, and Th1-derived cells from the spleens of tumor-bearing CD45.2⁺ WT B6 mice 12 days after transfer. The global transcriptional profile of the T cells was analyzed in duplicate by gene array. Analysis revealed that the Th9 cell gene expression profile was distinct from that of Th1 and Th17 cells (FIG. 2A). Strikingly, Th9 cells expressed higher levels of several costimulatory molecules (FIG. 2B). These 3 subsets of Th cells also differed in expression of effector molecules, transcriptional factors, and cytokines (FIGS. 2C-2D and FIGS. 9A-9B). Th1 cells highly expressed Th1-related transcriptional factors (Irf1, Stat1 and Tbx21) and several effector molecules (FasL and Granzyme [Grz] B), but did not express 112, suggesting a more terminally differentiated state. Particularly interesting is that Th9 cells had greater expression of Id2 and Eomes, transcriptional factors that suggest effector cell development, and increased expression of a Grz panel (GrzB, GrzD, GrzE, GrzG and GrzN). On the other hand, transferred Th9 cells also showed higher expression of Id3 and 112, which suggests that they are neither terminally differentiated nor short-lived. Finally, we found that Bach2, which promotes the differentiation of long-lived memory cells and Treg cells but restrains effector cell development, was highly expressed only in Th17 cells (FIG. 2C), confirming the memory feature and reduced cytolytic function of Th17 cells. The increased Bach2 expression in Th17 cells may also account for their partial conversion into Foxp3⁺ Treg-like cells in vivo (FIG. 9C), because Bach2 is known to promote formation and stabilization of Treg cells.

To more accurately assess effector T cell development of the Th9 cells in an unbiased manner, we performed gene set enrichment analysis (GSEA) to generate an enrichment plot for a mature T cell effector gene signature set. GSEA of the gene array data obtained from Th cells 12 days after transfer revealed that the mature effector gene signature was significantly enriched in both Th9 and Th1 cells but not in Th17 cells, and enrichment did not differ between Th1 and Th9 cells (FIG. 2E). These results again suggest that Th9 may be equal to Th1 cells in terms of mature effector T cell development. Intriguingly before transfer, significantly upregulated Eomes expression could be detected in Th9 cells by RT-PCR and intracellular staining (ICS) which was even greater than that in classic cytolytic Th1 cells (FIGS. 2F-2H). As Eomes is the effector master regulator that controls granzyme expression, Th9 cells also expressed markedly increased GrzA, GrzD, and GrzK among the tested T cell subsets and expressed a similar level of GrzB as compared with Th1 cells (FIG. 211). The Th9 Eomes and Grz expression patterns prompted us to directly test the cytolytic function of these cells. As shown in FIG. 2I and FIG. 9D, Th9 cells generated in vitro and sorted from tumor-bearing mice 12 days after transfer had the highest tumor-specific killing activity as compared with Th1, Th17, and other Th cells. We also observed that Th9-mediated specific killing was primarily granzyme-dependent, and particularly required granzyme B activity (FIGS. 9D-9F). Our data thus far indicate that Th9 cells display a core molecular signature consistent with programming as mature effector T cells.

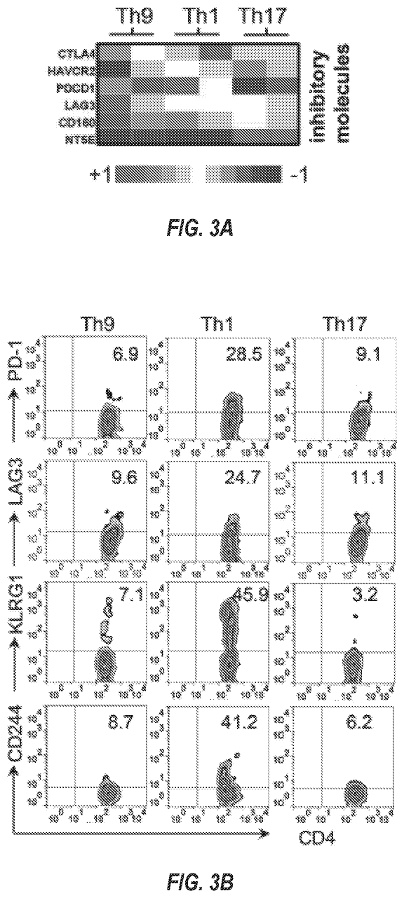

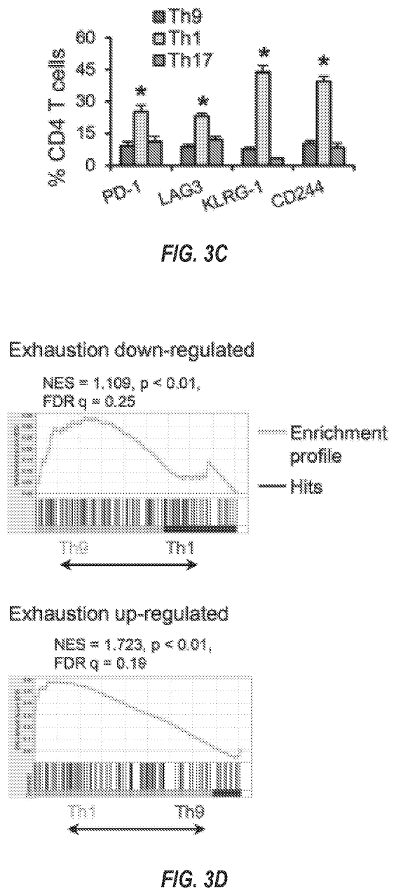

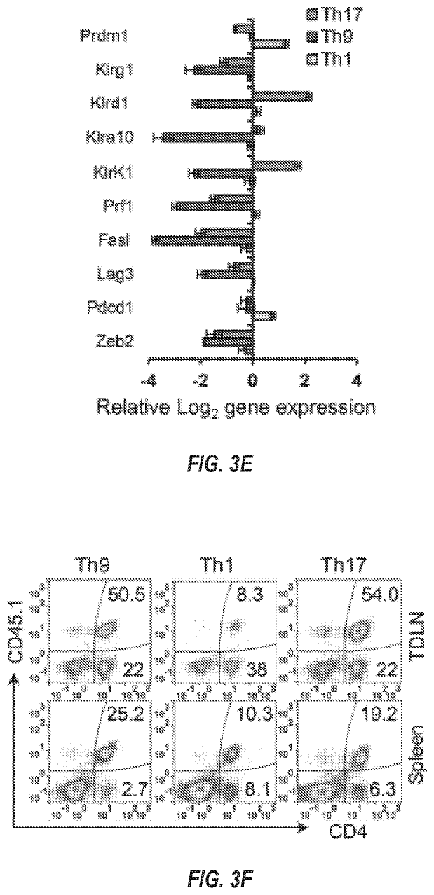

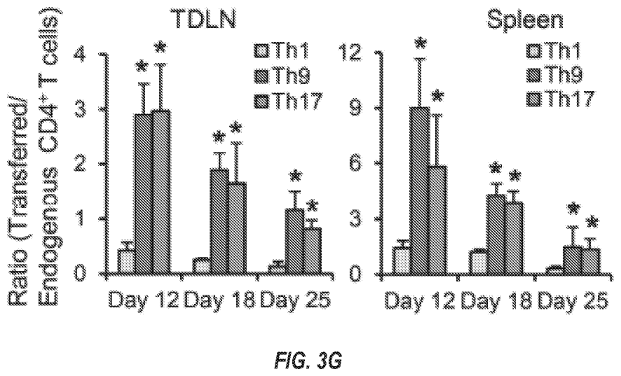

Th9 cells do not display exhausted or terminally differentiated T cell phenotype. Classic cytolytic Th1 cells display exhausted profiles, which greatly limits their antitumor function. Although our results suggested Th9 cells to be distinct effector cells, we wondered whether Th9 cells also display an exhaustion feature. Gene array data analysis showed that Th9 cells at 12 days after transfer expressed the lowest levels of inhibitory receptors (Ctla4, Havcr2 [Tim-3], Pdcd1, Lag3, Cd160, and Nt5e [CD73], FIG. 3A). As confirmation, FACS indicated that only Th1 cells upregulated these molecules, including PD-1, Lag3, KLRG-1, and CD244 (FIGS. 3B-3C). We further assessed the Th9 exhaustion profile by GSEA. We found that Th9 cells were significantly enriched in the exhaustion-downregulated gene signature, whereas Th1 cells were significantly enriched in an exhaustion-upregulated gene signature (FIG. 3D), suggesting that Th1 cells, but not Th9 cells, carry the molecular signature of the T cell exhaustion phenotype.

High T-bet expression is closely associated with terminal differentiation and drives short-lived T cell development, which seriously hampers the antitumor potential of Th1 cells. Because Th9 cells had low T-bet but high IL-2 expression (FIGS. 2C-2D), we hypothesized that Th9 cells are not terminally differentiated or late-stage short-lived Th1-like cells. Indeed, only polarized Th1 cells had increased expression of Prdm1 and Klrg1, the hallmarks of terminal differentiation and T cell senescence, respectively (FIG. 3E). Moreover, Th1 but not Th9 or Th17 cells highly expressed inhibitory molecules and other end-effector function markers (Klrd1, Klra10, KlrK1, Prf1, Fas1, Lag3, Pdcd1, and Zeb2, FIG. 3E) that have been reported to be associated with terminal differentiation, consistent with their lower antitumor activity in vivo.

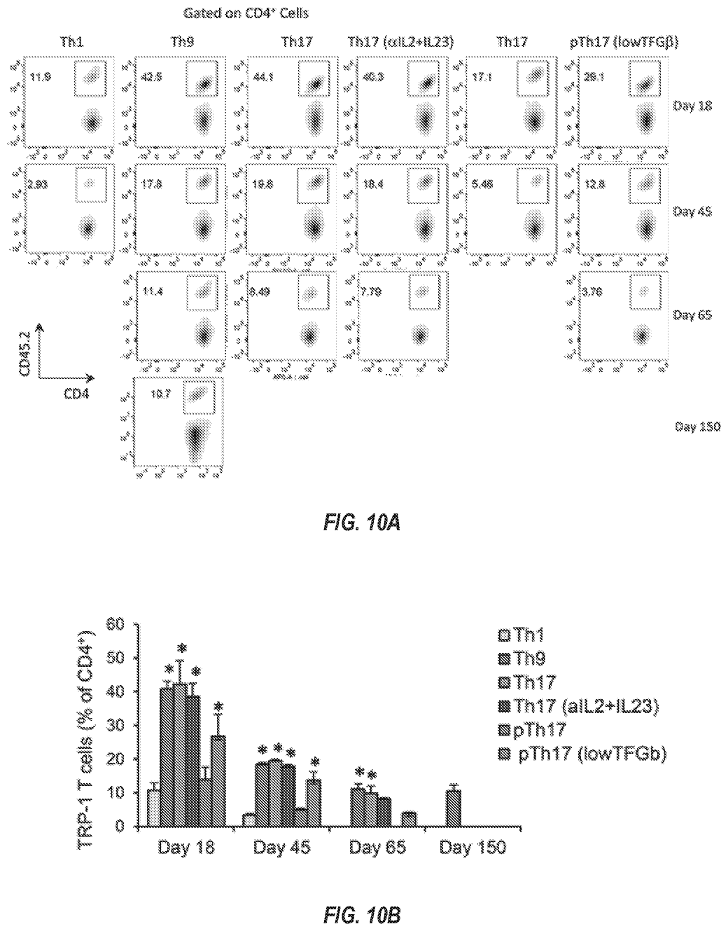

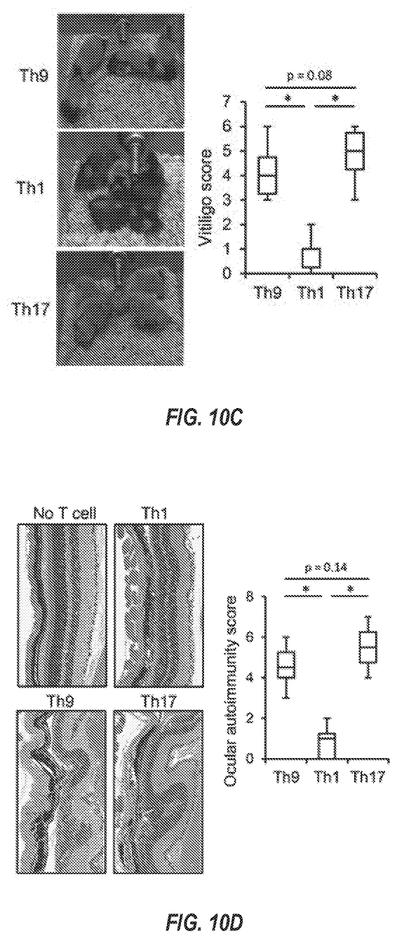

T cell subsets possessing great persistence are essential for successful ACT, which is the key reason why long-lived Th17 cells outperform terminally differentiated short-lived Th1 cells. We, therefore, determined the persistence capacity of these less exhausted effector Th9 cells. Noticeably, Th1 cells had the lowest number of surviving transferred cells in spleen and tumor-draining lymph nodes (TDLNs) over time (FIGS. 3F-3G and FIGS. 10A-10B), confirming their short-lived terminally differentiated signature. In striking contrast, Th9 cells had extraordinary persistence equal to, if not better than, the “stem cell-like” early memory Th17 cells (FIGS. 3F-3G and FIGS. 10A-10B). The long-term persistence of TRP-1 Th9 and Th17 cells also resulted in far greater autoimmune phenomena than Th1 cells, including the development of vitiligo and uveitis (FIGS. 10C-10D). Thus, Th9 cells may represent an unidentified effector T cell phenotype that is distinct from the classic cytolytic Th1 cells: a less exhausted fully cytolytic effector function and exceptional persistence after transfer.

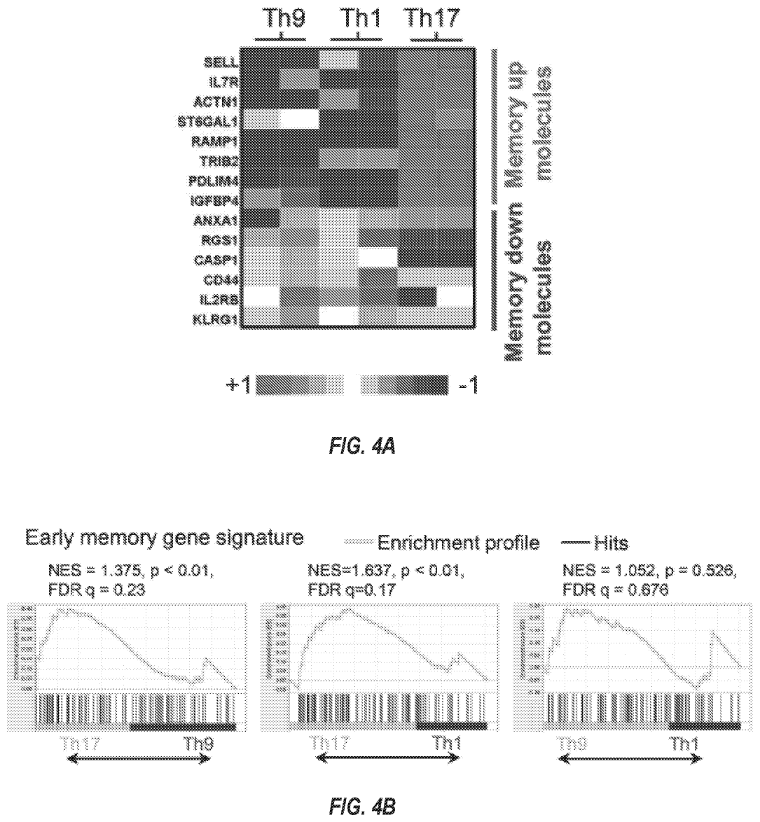

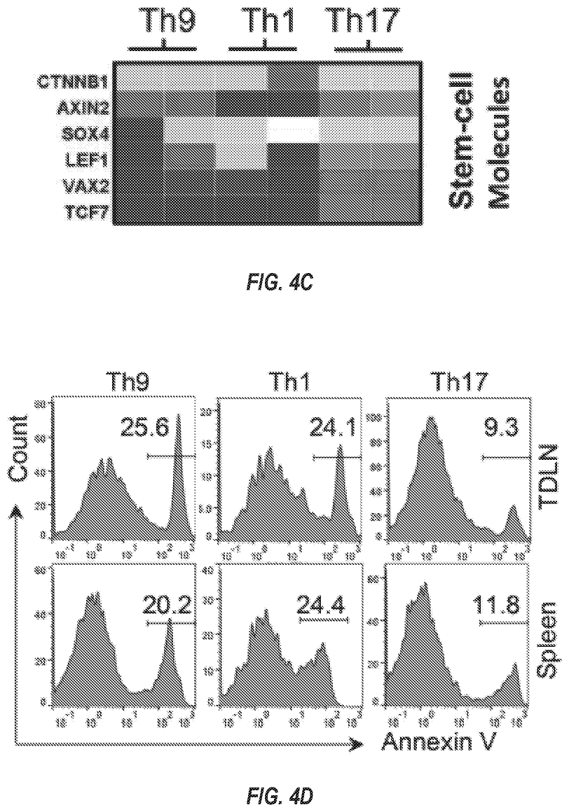

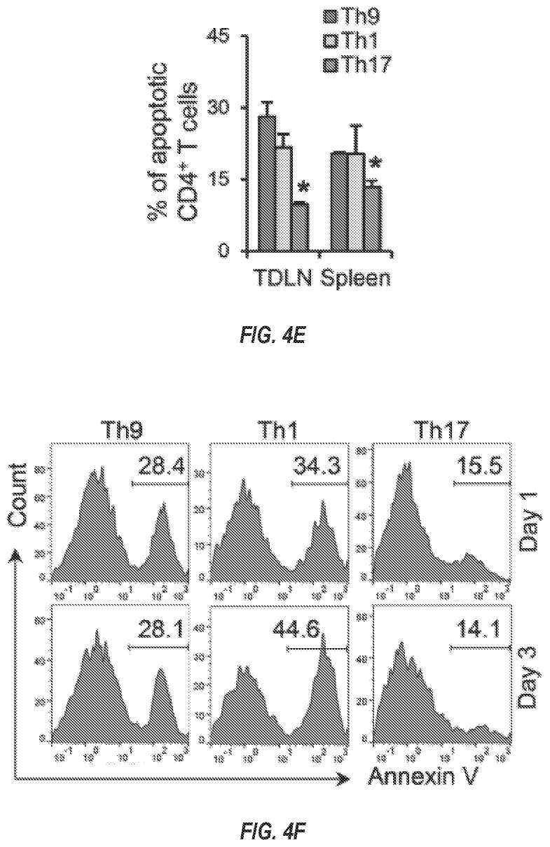

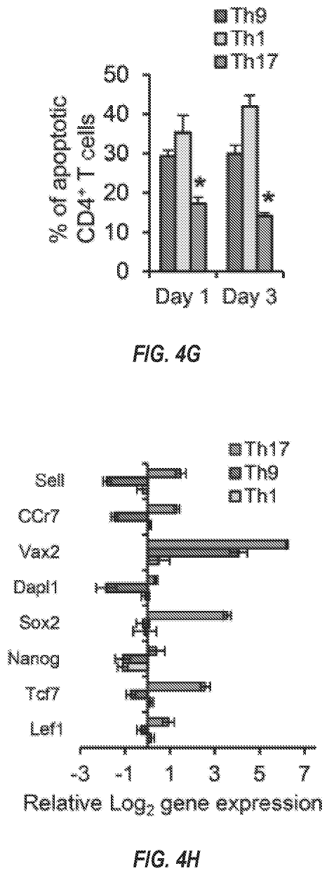

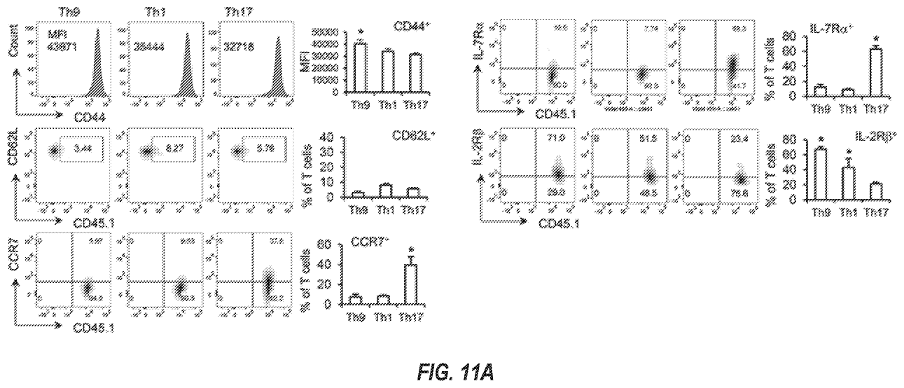

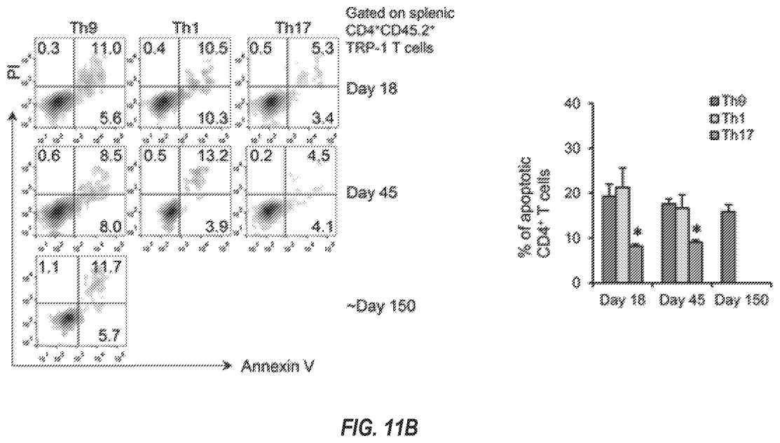

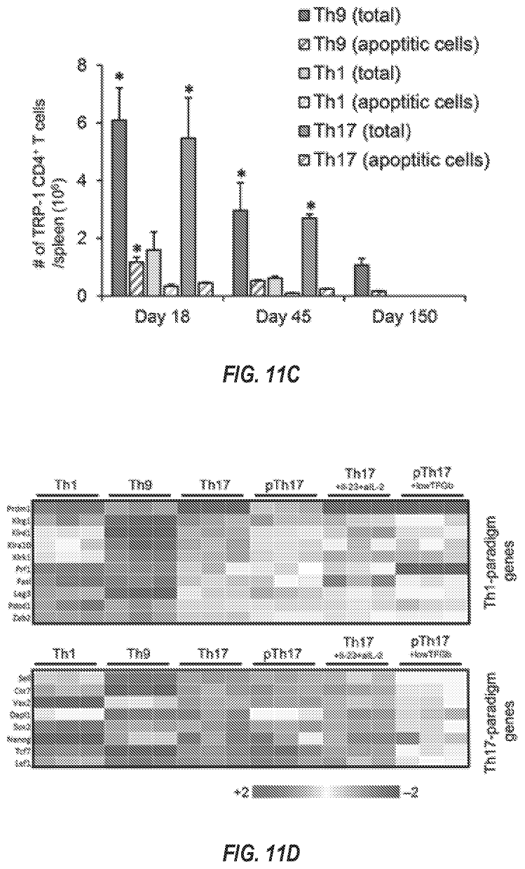

Th9 cells do not have memory or stem cell-like features. Early memory T cells are classically associated with prolonged peripheral persistence after ACT, so we first hypothesized that Th9 cells may also fit into this early memory classification. However, analysis of the gene expression profile that governs early memory development suggested that only Th17 cells display a core molecular signature of a less differentiated memory subset (FIG. 4A). This was confirmed by FACS analysis of some commonly used phenotypic markers of memory T cells (FIG. 11A). GSEA further demonstrated that as compared with Th9 or Th1 cells, Th17 cells were significantly enriched in features characteristic of memory precursor cells that survive and give rise to long-lived memory cells (FIG. 4B). In contrast, Th9 cells resembled the Th1 effector-type T cells, which were skewed away from early memory lineage development (FIGS. 4A-4B).

Acquisition of “sternness” can also allow transferred T cells to persist long-term, so we next hypothesized that Th9 cells may be “stem cell-like” T cells. We, therefore, analyzed the hallmark gene targets of the Wnt-β-catenin signaling axis (e.g. Ctnnb1, Axin2, Sox4, Lef1, Vax2, and Tc7), a pathway required for the maintenance of sternness in T cells. As shown in FIG. 4C, only Th17 cells expressed these sternness hallmark genes, confirming the previous observation of the stem cell-like nature of Th17 cells. We also assessed the central sternness functional properties of these T cell subsets by analyzing their resistance to apoptosis. The results showed that the apoptotic rate of Th9 cells was similar to that of Th1 cells in both spleen and tumor-draining lymph nodes (TDLNs) (FIGS. 4D-4E and FIGS. 11B-11C). In addition, when restimulating the in vitro-differentiated Th cells with antigen-pulsed APCs, we observed that Th9 cells still had no greater antiapoptotic capacity than Th1 cells (FIGS. 4F-4G). On the other hand, Th17 cells demonstrated the lowest apoptotic rate both in vivo and in vitro, which is consistent with their early memory/stem cell-like properties (FIGS. 4D-4G).

Indeed, these gene expression patterns existed before Th9 cell transfer, as shown in FIG. 4H and FIG. 11D. After polarization in vitro for 5 days, we detected by RT-PCR that Th9 cells existed as a transcriptionally distinct population: they had lower expression levels (much lower than even short-lived Th1 cells) of genes for memory markers (Sell and Ccr7), early T cell development (Vax2 and Dapl1), and sternness (Sox2, Nanog, Tcf7, Lef1). Considering that Th9 cells also expressed the lowest level of terminally differentiated end-effector function markers (even much lower than long-lived Th17 cells, see FIG. 3E), it appears that the current understanding of a stem cell/early memory Th17 paradigm versus a terminal/end effector Th1 paradigm is insufficient to explain the exceptional persistent capacity and antitumor effectiveness of Th9 cells.

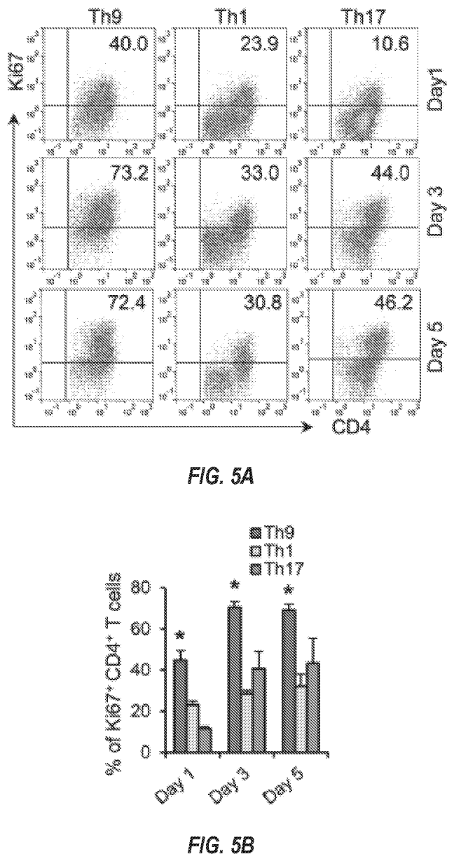

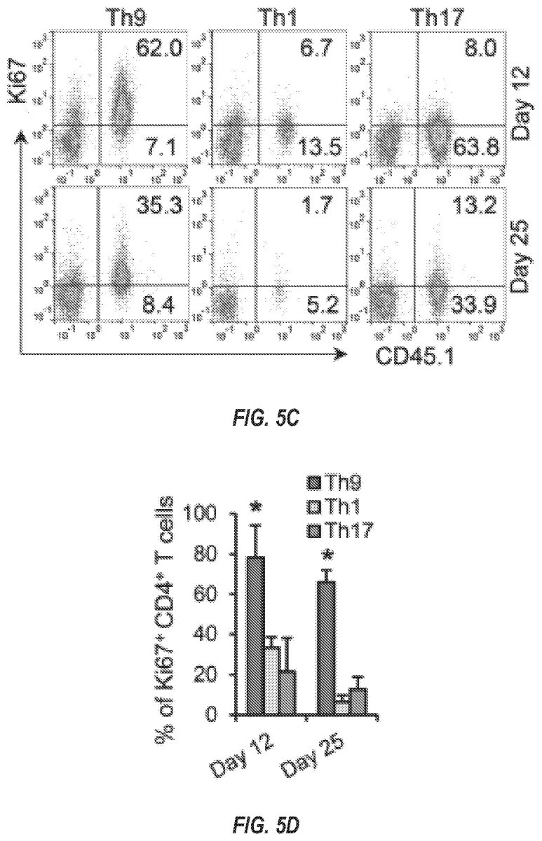

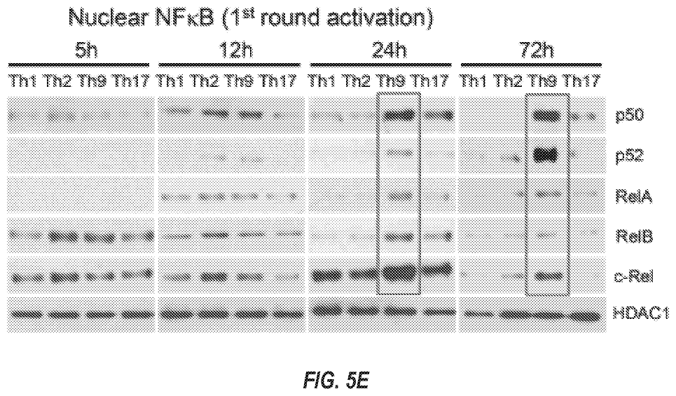

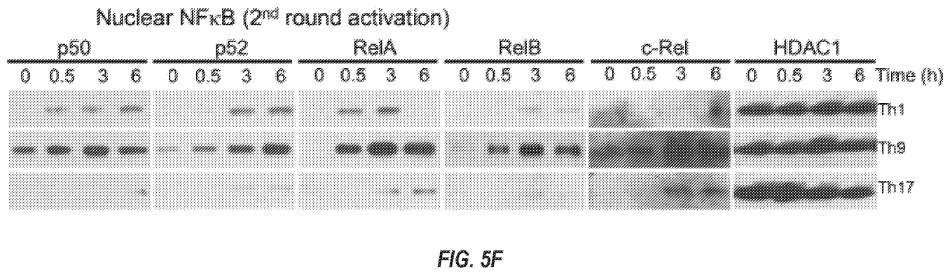

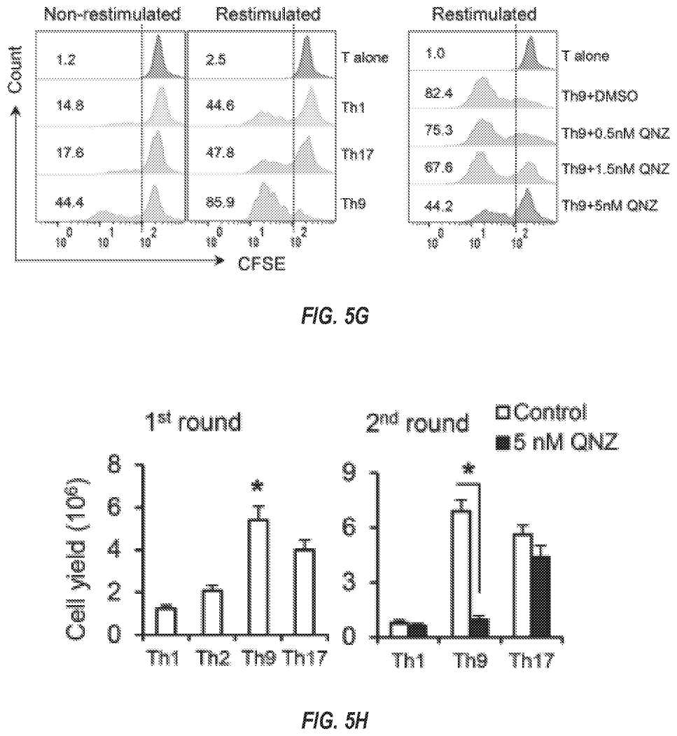

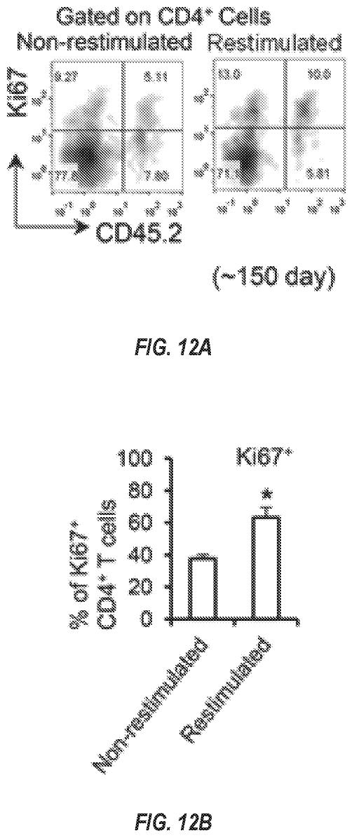

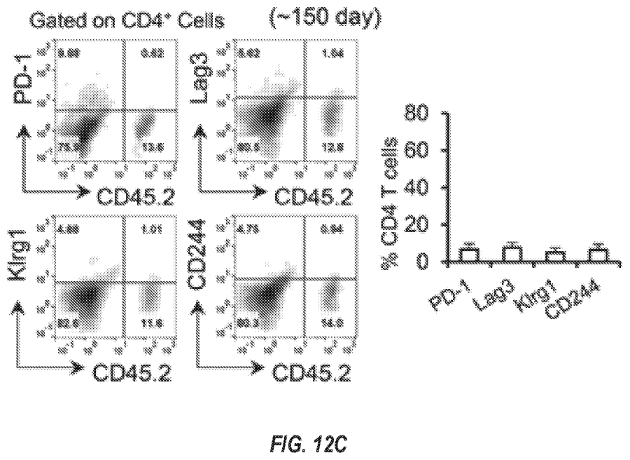

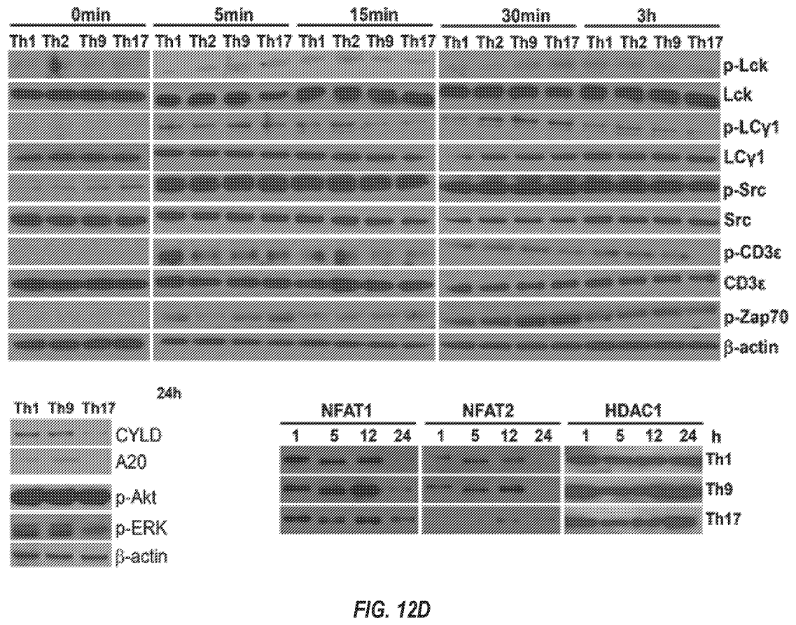

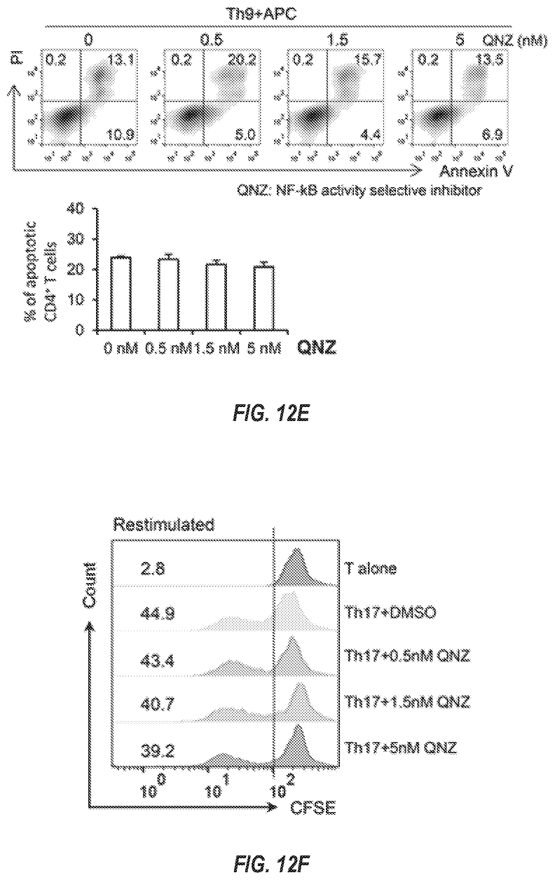

Th9 cells display a hyperproliferative feature mediated by the hyperactivation of late-phase NFκB signaling. Because Th9 cells do not seem to have an enhanced antiapoptotic advantage, and the current literature does not clearly explain their prolonged persistence after transfer, we sought to determine Th9 proliferative capacity. We first reactivated Th1, Th9, and Th17 cells with antigen-pulsed APCs in vitro, and assessed Ki67 expression as a readout for proliferating cells. Surprisingly, the percentage of Ki67⁺ cells was significantly greater in Th9 cells as compared with Th1 and Th17 cells (FIGS. 5A-5B). To verify our finding, we also assessed Th9 cell proliferation over time in tumor-bearing mice. We found a large population (>80% on day 12 and ˜70% on day 25) of proliferating OT-II-Th9 cells in the TDLNs, whereas Th1 and Th17 cells showed limited proliferation over time (FIGS. 5C-5D). Moreover, 150 days after transfer of TRP-1-Th9 cells, 10% of splenic CD4⁺ T cells in the mice were transferred Th9 cell-derived cells that exhibited cytolytic activity (FIG. 8G), a less exhausted profile, and greater proliferation (FIGS. 12A-12C). These results suggested that Th9 cells possess a unique hyperproliferative advantage over other antitumor T helper cells, which may be responsible for the superior antitumor features of Th9 cells.

Next, we comprehensively analyzed the T cell receptor (TCR)-signaling in Th9 and other Th cell subsets. Upon activation, we noted that all these Th cells displayed similar TCR-proximal signaling events, including phosphorylation of the protein tyrosine kinases Lck, LCy1, Src, and Zap70, and their downstream signaling events, such as the MAP kinase Erk, the kinase Akt, and nuclear translocation of NFAT (FIG. 12D).

Because NFκB signaling is pivotal in controlling T cell proliferation, we systematically analyzed NFκB signaling activation in Th cells. First, we detected no decrease in cytosolic proteins that are involved in negative regulation of TCR-to-NFκB signaling (such as A20 and CYLD) in Th9 cells (FIG. 12D). However, a striking difference is the hyperactivation of NFκB, detected by nuclear translocation of p50, RelA, RelB, p52, and c-Rel in Th9 but not in Th1, Th2, or Th17 cells. This occurred at late time points after cell stimulation (24 h-72 h), whereas during the early phase (<12 h), NFκB activation was similar across all T cell subsets assessed (FIG. 5E). These results highlight that Th9 cells possess a unique hyperproliferative feature, possibly mediated by hyperactivation of late-phase NFκB. We also found a similar re-hyperactivation of NFκB signaling only in Th9 cells when they were restimulated with anti-CD3 plus anti-CD28 mAbs on day 5 after the first-round activation (FIG. 5F).

Moreover, we further confirmed the increased proliferative capacity of Th9 cells by CFSE-dilution assay and by calculating the cell yields after the first-round activation and the subsequent reactivation (FIGS. 5G-5H). Importantly, inhibition of NFκB signaling by a specific inhibitor (QNZ) did not induce Th9 cell apoptosis (FIG. 12E), but significantly arrested Th9 hyperproliferation (FIG. 5G-5H) with minimal effect on proliferation of Th17 (FIG. 12F). Taken together, these data indicate that the hyperactivation of late-phase NFκB drives the hyperproliferation of Th9 cells, a unique feature that has not been described in other known Th cells.

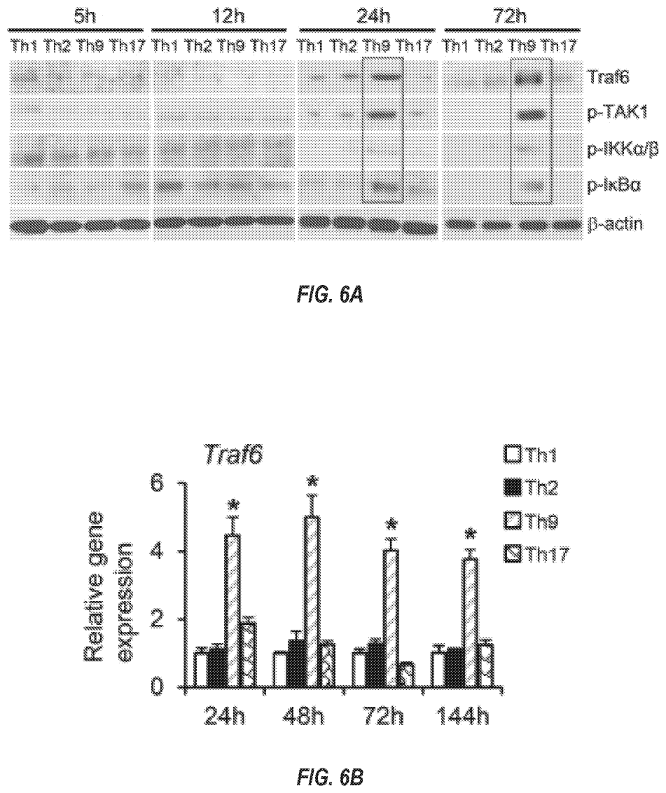

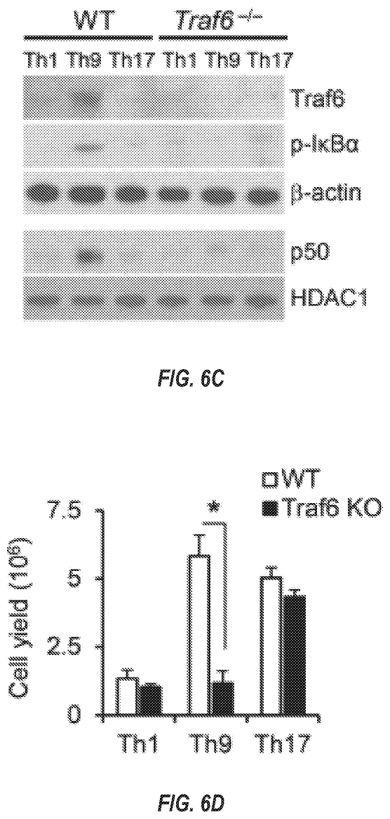

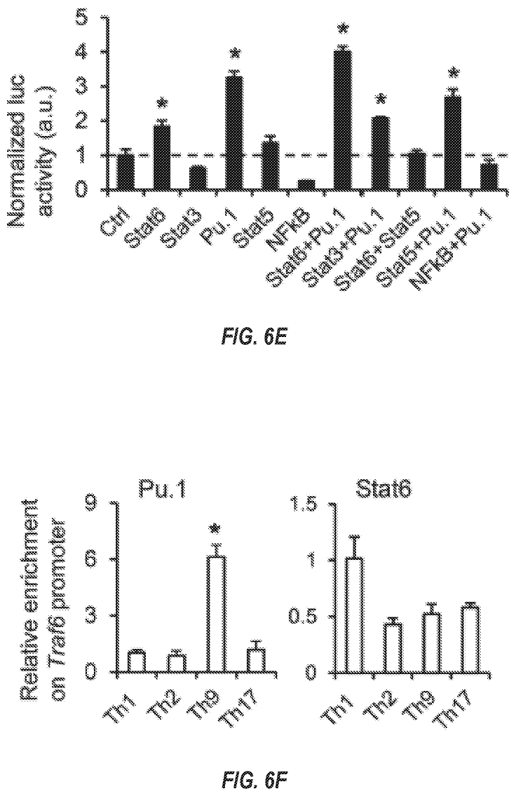

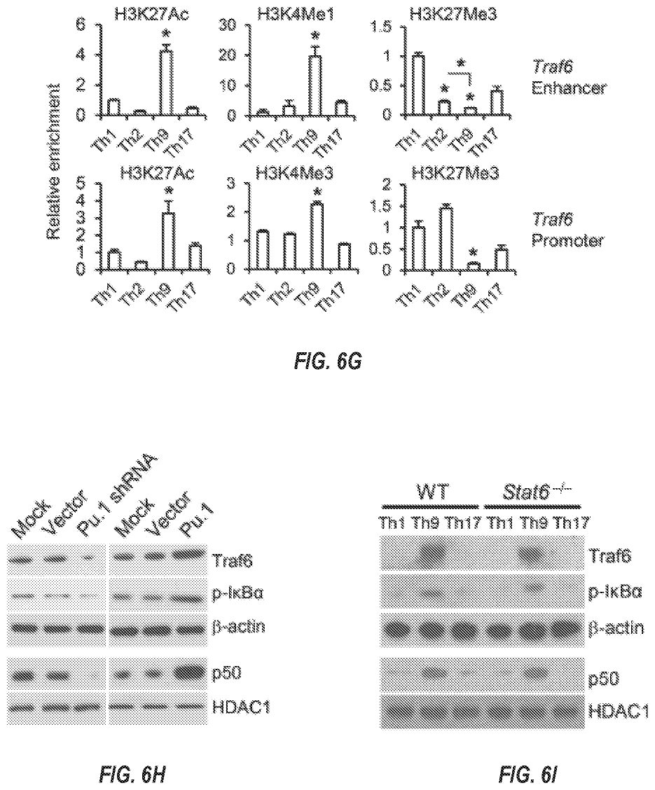

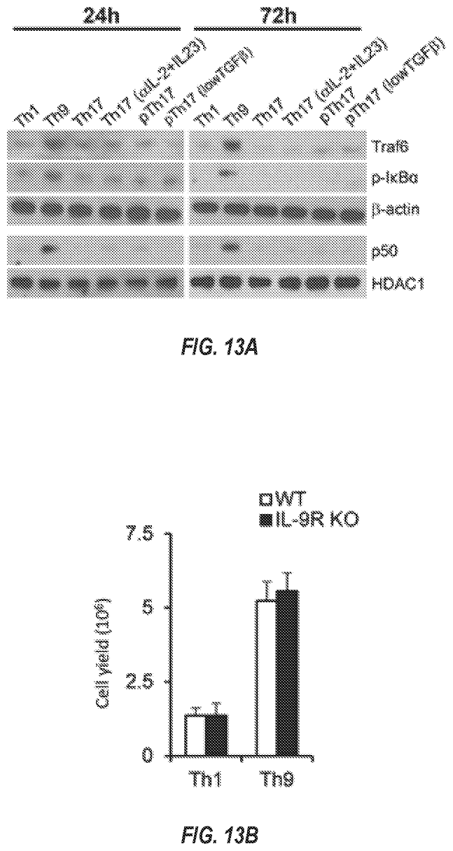

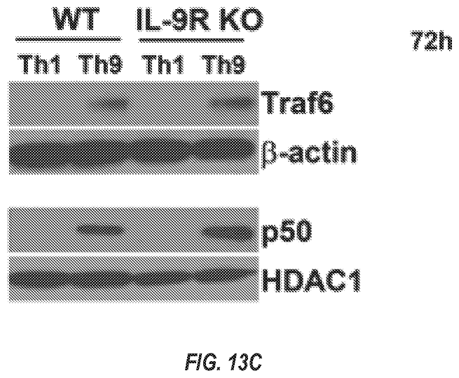

Increased Traf6 production drives hyperactivation of NFκB signaling to promote Th9 hyperproliferation. To understand the mechanism that drives the late-phase NFκB hyperactivation in Th9 cells, we systematically analyzed the NFκB signaling activation in these cells. Although no significant changes occurred in regard to the TCR-proximal signaling events and cytosolic proteins that are involved in negative regulation of TCR-to-NFκB signaling, NFκB upstream signaling proteins (Traf6, pTAK1, pIKKα/β, pIκBα) levels substantially increased in Th9 cells (FIG. 6A and FIG. 13A). This intriguing difference highlights that Traf6 might be responsible for the hyperactivation of late-phase NFκB because both Traf6 protein and mRNA were upregulated significantly (FIGS. 6A-6B), and the recruitment of Traf proteins is a key step in the activation of NFκB in T cells. To test the importance of Traf6 in Th9 cells, we generated Traf6^{-/-}-Th9 cells from Traf6^{flox/flox}CD4^{cre} mice. Traf6-deficiency completely abolished hyperactivation of NFκB signaling and the hyperproliferation of Th9 cells (FIGS. 6C-6D), further demonstrating that Traf6 is critical in regulating the hyperproliferative feature of these cells.

These results also prompted us to determine what regulates Traf6 upregulation in Th9 cells. By analyzing the promoter region of Traf6, we predicted several binding sites for transcription factors, such as Stat3, Stat5, Stat6, Pu.1 (Spi1), and NFκB, that might have been activated in Th9 cells (Table 2). To determine whether these molecules could activate the Traf6 promoter, we performed luciferase reporter assays. Stat3, Stat5 and NFκB did not activate the Traf6 promoter, whereas Pu.1, and to a lesser extent, Stat6 did (FIG. 6E). Considering that Pu.1 and Stat6 are crucially involved in Th9 cell development, we performed a chromatin immunoprecipitation (ChIP) assay. As shown in FIG. 6F, we observed that only Pu.1 bound the Traf6 promoter region in Th9 cells. To gain further insight, we determined epigenetic changes at the Traf6 locus and observed striking differences in the acetylation and methylation status (FIG. 6G), suggesting active Traf6 enhancer and promoter regions in Th9 cells. Specifically, the “permissive” histone marks (H3k4Me1 on enhancer, H3k4Me3 on promoter, and H3K27Ac on both enhancer and promoter) were highly increased on the Th9 Traf6 locus. Conversely, Th9 cells had the least H3K27 trimethylation (H3K27Me3) on both the enhancer and promoter of Traf6, which is a ‘non-permissive” histone mark associated with repressed genes (FIG. 6G). These chromatin modifications might affect Traf6 locus accessibility to transcription factors, such as Pu.1, in Th9 cells.

To obtain direct evidence that Pu.1 contributes to Traf6 production in Th9 cells, we compared Traf6 expression levels in WT-Th9, Ctrl-shRNA-transduced Th9, Pu.1-shRNA-transduced Th9, GFP-retrovector-transduced Th9, Pu.1-retrovector-tranduced Th9, Il9r^{-/-}-Th9, and Stat6^{-/-}-Th9 cells. Results clearly showed that Traf6 levels and NFκB signaling were significantly upregulated in Th9 cells overexpressing Pu.1 and downregulated in Pu.1 knockdown (KD) Th9 cells (FIG. 611). By contrast, in Stat6^{-/-}-Th9 and Il9r^{-/-}-Th9 cells, the Traf6 level was similar to that in WT-Th9 cells (FIG. 6I and FIGS. 13B-13C). These data pinpoint the importance of Pu.1 in transcription of Traf6 in Th9 cells, whereas IL-9 signaling seems not to be required for their Traf6 expression, NFκB signaling, or hyperproliferation.

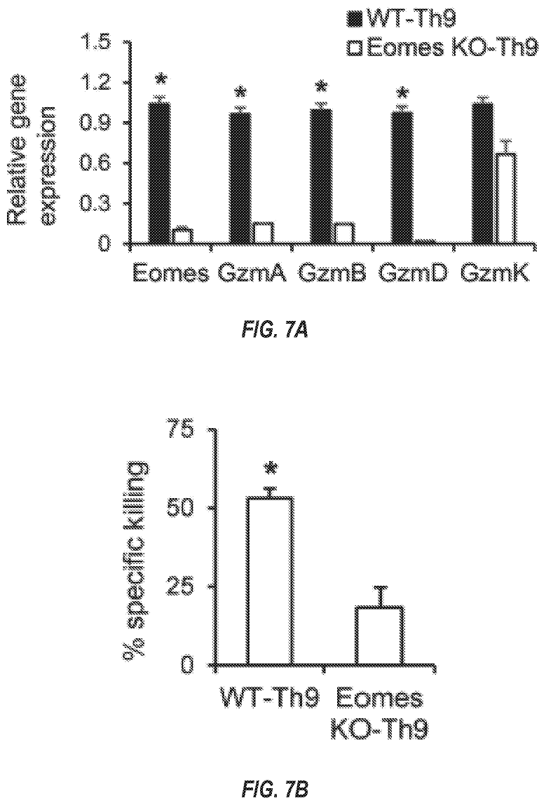

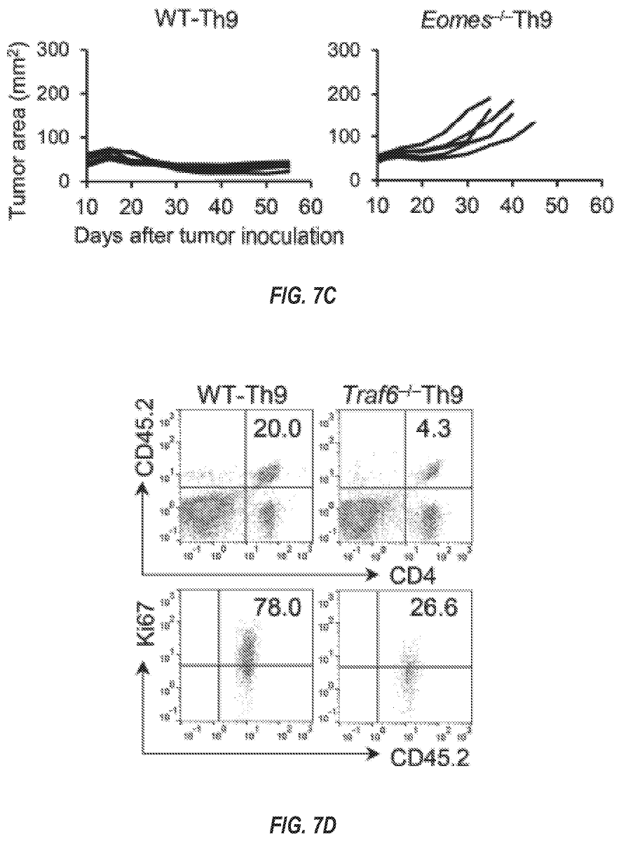

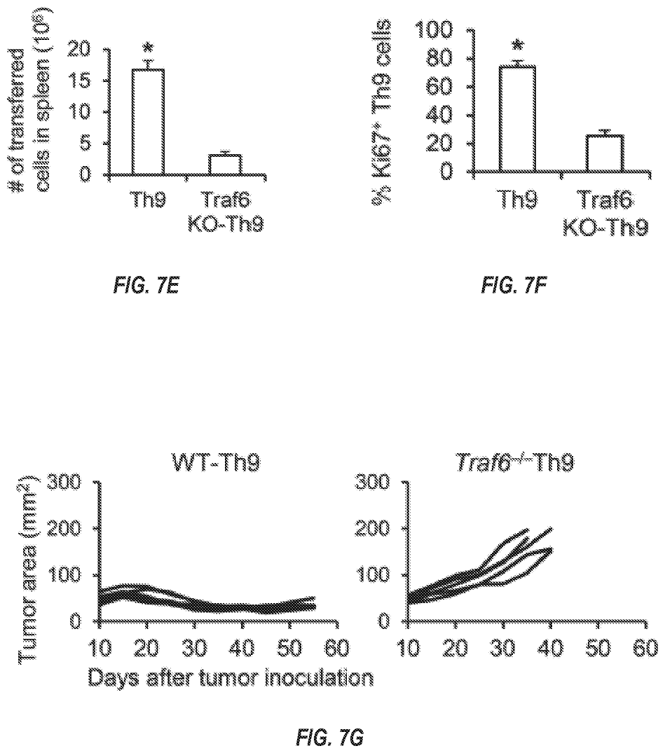

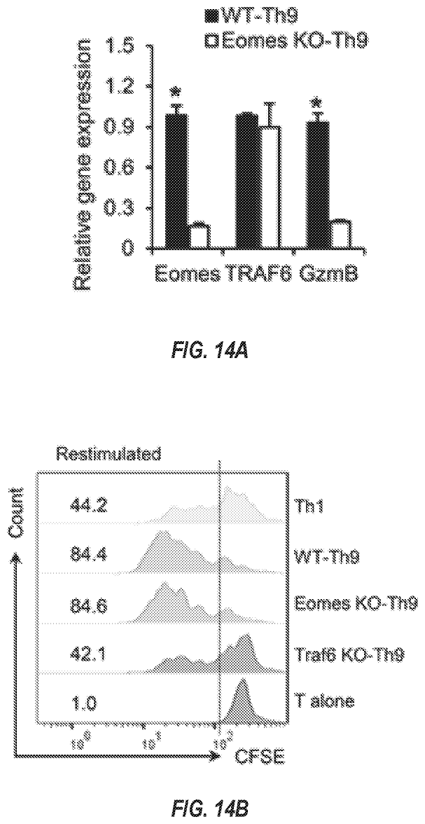

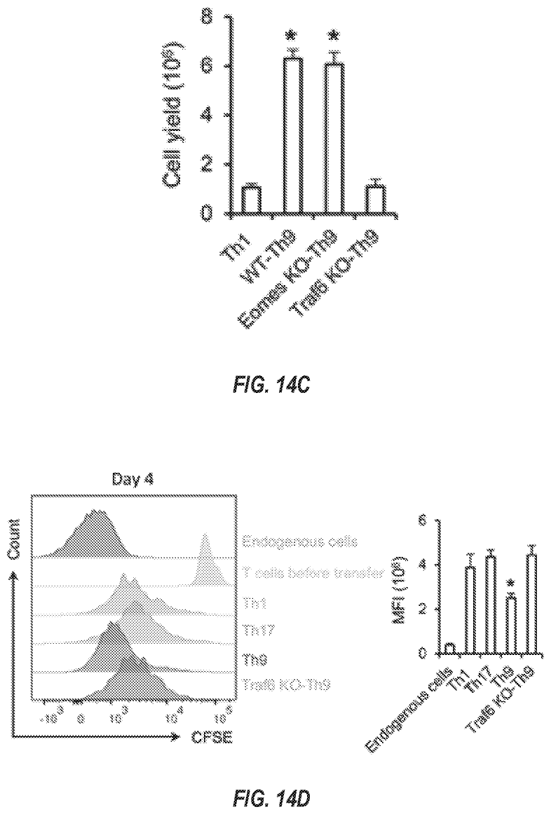

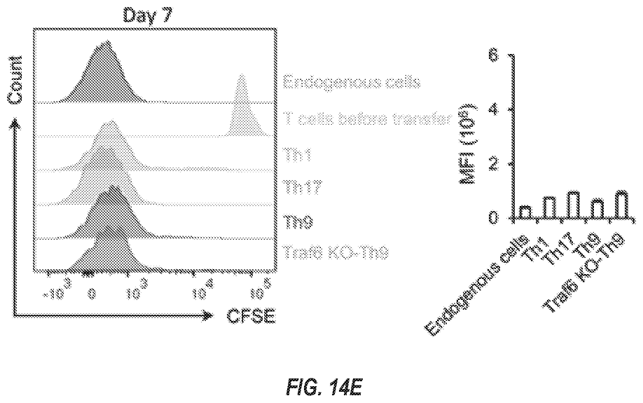

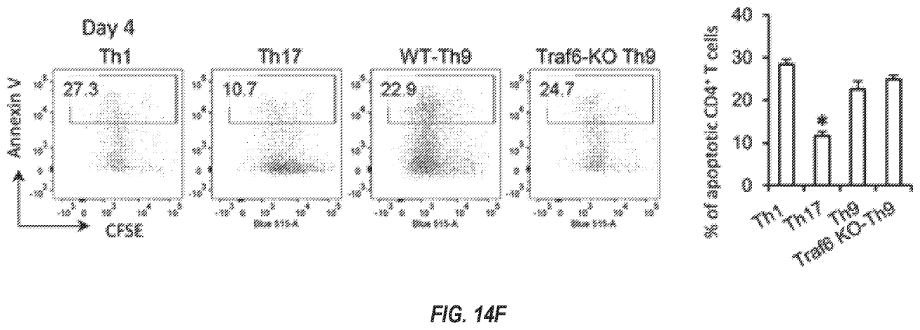

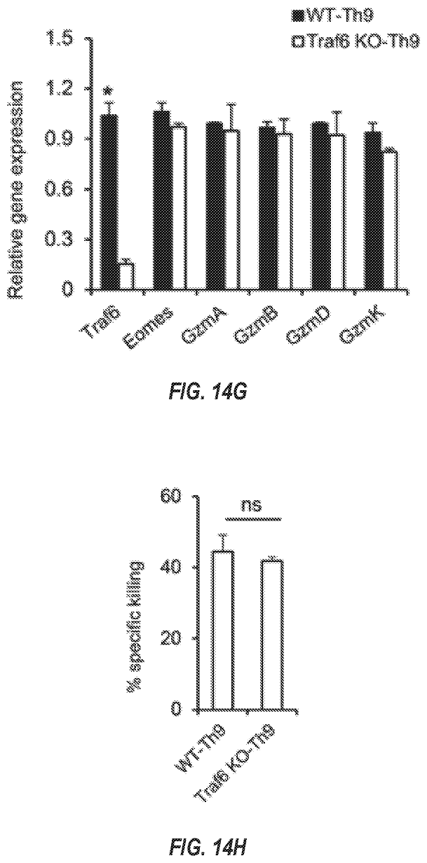

Eomes and Traf6 dictate the antitumor efficacy of Th9 cells. To ascertain the contributions of the effector and hyperproliferative properties of Th9 cells in the Th9-mediated eradication of large established tumors, we examined key factors involved in determining the feature and function of Th9 cells. First, we further dissected the role of the effector master transcriptional factor Eomes in Th9 cells. Eomes deficiency abolished granzyme expression by Th9 cells and subsequently extinguished their cytolytic activity in a direct in vitro killing assay (FIGS. 7A-7B), but did not significantly change their Traf6 expression and proliferative capacity (FIGS. 14A-14C). Consistent with these findings, Eomes^{-/-}-Th9 cell transfer failed to mediate sustained antitumor responses as compared with WT-Th9 cells (FIG. 7C). In addition, we investigated whether Traf6 was essential for the superior antitumor performance of Th9 cells. We observed significantly lower frequencies and decreased proliferation of Traf6^{-/-}-Th9 cells after transfer (FIGS. 7D-7F and FIGS. 14D-14F), and this insufficient persistence of Traf6^{-/-}-Th9 cells also nullified their antitumor ability without altering their cytolytic function (FIG. 7G and FIGS. 14G-14H). This effect appeared to apply only to Th9 cells because anti-tumor function was similar between WT-Th17 and Traf6^{-/-}-Th17 cells in vivo (FIG. 14I). Collectively, our data provide functional confirmation of the observed molecular program of effector development and hyperproliferation displayed by tumor-specific Th9 cells.

The present study has revealed cellular and molecular mechanisms by which tumor-specific Th9 cells promote tumor regression in a highly realistic and clinically relevant ACT scenario.

Two paradigms have emerged in determining the functionality of T cells for ACT, based on the fact that antitumor efficacy inversely correlates with advanced maturational state through limitation of the capacity to self-renew and survive in vivo. The Th1 paradigm focuses on the terminal effectors prone to apoptosis, whereas the Th17 paradigm focuses on less differentiated subsets capable of superior persistence and functionality in vivo. Taking into consideration the global gene expression profile, we used GSEA to determine the phenotypic features of Th9 cells by comparing their characteristics to those of these two existing T cell paradigms. Regarding the effector maturational status, GSEA suggested that a core molecular signature might be shared between Th9 and Th1 cells. In agreement with this result, Th9 cells generated in vitro and sorted from tumor-bearing mice always had the highest tumor-specific killing activity. Intriguingly, although Th9 cells are mature effector T cells, their phenotype is distinct from that of the classic Th1 effectors. First, GSEA suggested that unlike Th1 cells, Th9 cells are not enriched in the molecular exhaustion signature. Second, unlike Th1 cells, Th9 cells do not display the features of terminally differentiated late effectors. Third and most strikingly, Th9 cells show extraordinary persistence whereas Th1 cells are short-lived. These observed differences might be explained by Eomes upregulation, rather than T-bet, that drives the effector development of Th9 cells. On the contrary, high expression of T-bet, a master transcriptional factor for Th1 cells, has been closely associated with terminal differentiation and drives the short-lived T cell development, which seriously hampers the antitumor potential of Th1 cells. In addition, we observed that significantly increased granzyme production was responsible for Th9-mediated killing. However, Th9 cells did not possess significantly upregulated perforin expression as compared with other Th cell subsets; it may be that Th9 cells have already produced enough perforin, or that Th9 cell-activated CD8⁺ T cells may produce perforin as an additional source. Thus, these results suggest Th9 and Th1 cells are two transcriptionally and phenotypically distinct effector populations and that Th9 cells can not be classified into the Th1 paradigm.

Prolonged persistence has been previously attributed only to less differentiated early memory T cells, and T cell subsets possessing great persistence is crucial for successful ACT. This is the key reason why long-lived Th17 cells outperform the terminally differentiated short-lived Th1 cells for ACT. Although Th9 cells persist equally well to early memory Th17 cells, GSEA showed that Th17 cells, but not Th9 or Th1 cells, retained the molecular signature of early memory T cells. Furthermore, Th9 cells did not acquire stemness, nor did they display enhanced resistance to apoptosis in vivo. This counterintuitive finding can be explained by the observed hyperproliferation of Th9 cells in vivo. Molecular mechanistic studies uncovered that Pu.1, an important transcription factor for Th9 cell development, bound and transcribed Traf6 in Th9 cells. Traf6 may serve as a critical adaptor molecule that links to the MALT1-CARMA1-Bcl-10 complex downstream of TCR, and may function directly or indirectly by forming a complex with Ubc13/Uev1a as a ubiquitin ligase in order to attach ubiquitin chains to target proteins, including itself and IKKγ, which enable the formation of complexes by recruiting TAB2/3-TAK1 and then continuously activate the NFκB signaling pathway. This Pu.1-Traf6-NFκB pathway pinpoints an alternative mechanism that drives the extraordinary persistence of Th9 cells, which differs from the antiapoptotic strategy seen in Th17 cells.

Although it is not clear whether the hyperproliferation-mediated persistence of T cells possesses any advantage over the antiapoptotic Th17 behavior, a potential explanation of the Th9 superior functionality over Th17 cells is that the Th17 cells may not have developed into fully mature effector T cells, whereas newly polarized and transferred Th9 cells all have high cytolytic activity. The plasticity of Th17 cells, on the one hand, allows a portion of transferred Th17 cells to convert into Th1-like effector cells. On the other hand, Th17-to-Treg conversion has been suggested, and a portion of Th17 cells also converted into Foxp3⁺ Treg-like cells upon transfer in our experimental conditions, possibly due to the upregulated expression of Bach2, which promotes efficient formation and stabilization of Treg cells but restrains effector cell development. These results suggest that the potential for “stem cell-like” CD4⁺ T cells to convert into Treg cells in vivo might negatively affect their antitumor efficacy.

Our work has revealed that tumor-specific Th9 cells have a less exhausted and long-lived effector profile. This Th9 paradigm may challenge our current understanding of T cell selection criteria for ACT: (1) pre-acquisition of a maturational effector state in vitro may not limit antitumor functionality in vivo; (2) capacity for long-term persistence may not be associated with stem cell-like or early memory properties; and (3) IFN-γ- and TNF-α-production may not be required from the transferred T cells. In the Th9 paradigm, T cells possess a unique phenotype that is a combination of Th1 cytolytic and Th17 stem cell-like persistence characteristics, and thus they have significant implications for the design of ACT therapies.

### Example 2. In Vivo Studies of Several Cancer Types

The methods of this invention, employing CD4⁺ Th9 cells and antigen-presenting cells (e.g., dendritic cells (DCs)) were applied to additional tumor types. Specifically, we utilized three different mouse cancer cell lines, each genetically modified to express ovalbumin (OVA). In all three cases the cancer cells were inoculated into syngeneic mice of strain C57BL/6 (B6) and tumors were allowed to grow for 9 days. On day 9 the tumor-bearing were mice were given one dose of cyclophosphamide (CTX, 200 mg/kg) to induce temporary lymphopenia On the next day (day 10 from tumor inoculation) the mice were inoculated with OT-II Th9 cells and peptide-loaded APCs, essentially as described in the case of treatment of the syngeneic melanoma B16-OVA. Inoculation with saline alone (PBS), without immune cells, served as a control. The murine cancers utilized in this study were: Lewis lung carcinoma cells (LL2-OVA) (Bertram and Janik. “Establishment of a cloned line of Lewis Lung Carcinoma cells adapted to cell culture” Cancer Lett 11:63-73 (1980)); MC-38 colon adenocarcinoma cells (Mc38-OVA) (Rosenberg et al. “A new approach to the adoptive immunotherapy of cancer with tumor-infiltrating lymphocytes” Science 233:1318-1321 (1986)); and Panc 02 pancreatic adenocarcinoma cells (Pan2-OVA) (Corbett et al. “Induction and chemotherapeutic response of two transplantable ductal adenocarcinomas of the pancreas in C57BL/6 mice” Cancer Res 44:717-726 (1984)).

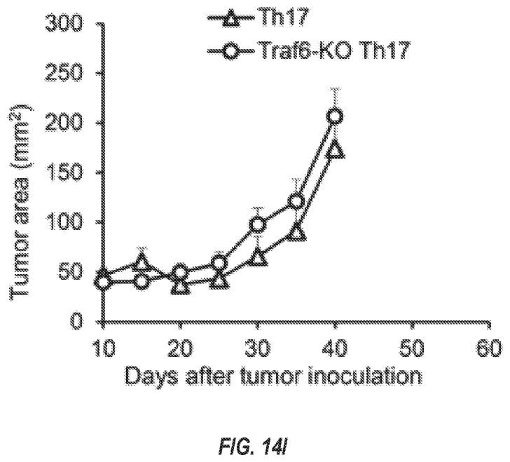

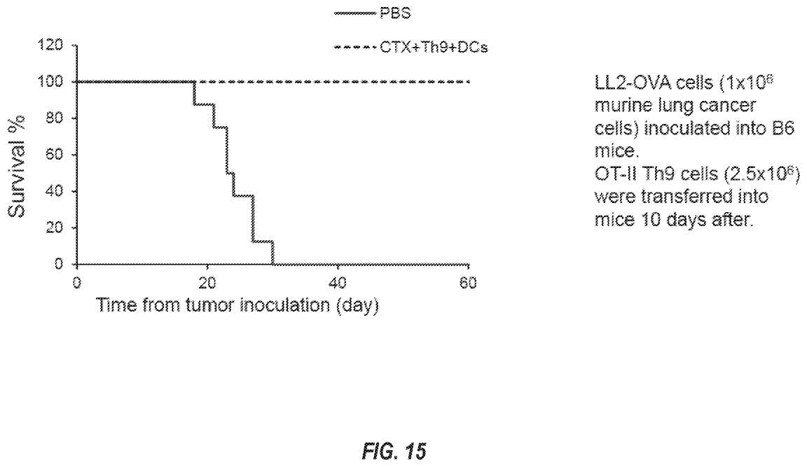

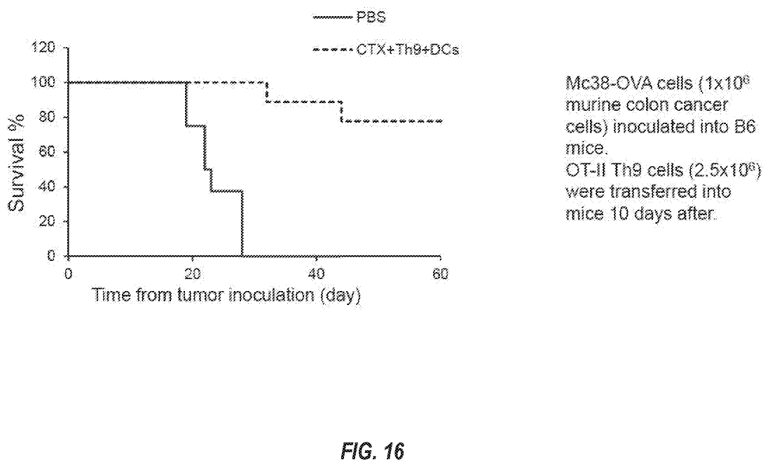

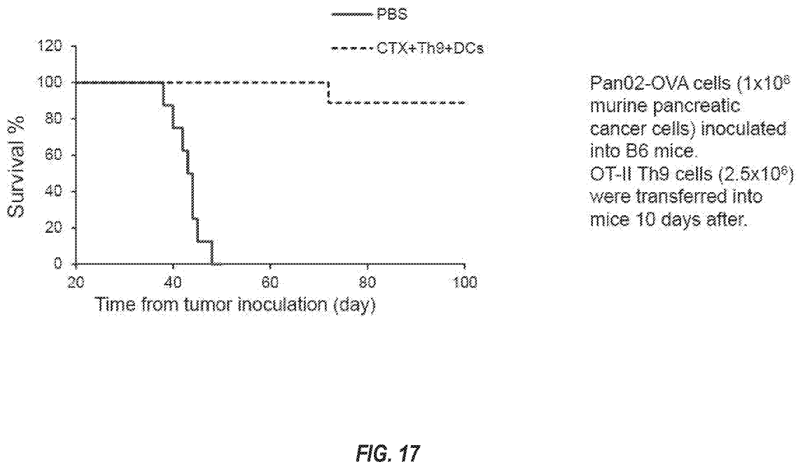

In each case, all animals that received neither adoptively transferred Th9 cells nor antigen-presenting cells reached criteria for euthanasia because of aggressive tumor progression by 30 to 50 days after cancer cell inoculation, depending on the tumor line. The mice treated with the complete therapeutic method of cyclophosphamide followed by Th9 adoptive cell transfer and dendritic cell vaccination (CTX+ Th9+DCs) showed dramatically improved survival. In the lung carcinoma model, all control (PBS) animals had to be euthanized by 30 days post-tumor cell inoculation, whereas all mice receiving the cell therapy remained viable at day 60 (FIG. 15), with complete tumor regression and no evidence of recurrence. Similarly, in the colon adenocarcinoma model, all control (PBS) animals required euthanization by day 30, while 7 of 9 treated animals remained alive and apparently tumor-free at day 60 (FIG. 16). Finally, in the pancreatic adenocarcinoma model, no control animal survived beyond day 50, whereas 8 of 9 treated mice were alive and free of detectable tumor at day 100 (FIG. 17).

These data show that the combination approach of cyclophosphamide to induce temporary lymphopenia plus antigen-specific cell therapy with Th9 and DC vaccinating cells can be used in multiple different cancers, in addition to melanoma. The excellent survival observed in mice with four different aggressive, metastatic syngeneic tumor cell lines, corresponding to melanoma, lung carcinoma, colon adenocarcinoma, and pancreatic adenocarcinoma, implies that a comparable therapeutic regimen should have value in the treatment of corresponding human cancers. The approach should be generalizable to any human cancer for which antigen-specific and/or tumor-specific populations of Th9 and antigen-presenting cells can be produced.

Another context in which adaptive cell therapy (ACT) with CD4⁺ Th9 cells might be valuable to treat cancer would be when the Th9 cells are engineered to express a chimeric antigen receptor (CAR, also known as a chimeric or artificial T cell receptor, abbreviated CAR T). We sought to compare the cancer-killing ability in vivo of human Th9 cells carrying a specific CAR with that of a mixture of human Th1 plus Tc1 cells carrying the same CAR, namely, one specific for the CD19 cell surface antigen expressed by B lymphocytes and B cell lineage lymphomas and leukemias.

The combined CAR-Th1+CAR-Tc1 cell population is currently being utilized in human cancer therapy.

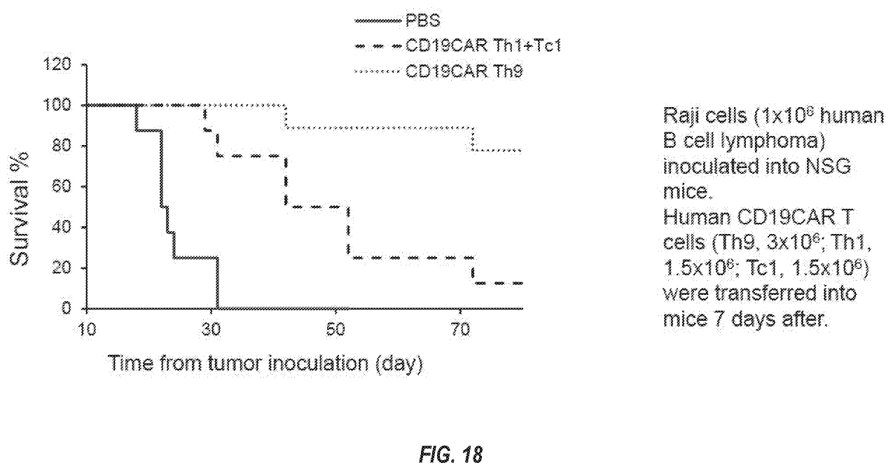

FIG. 18 shows our unexpected finding that ACT with Th9 cells engineered to express a CAR T (designed CAR-Th9) at an equal total cell dose provides significantly superior survival than the mixture of Th1 and Tel cells expressing the same CAR T (designated CAR-Th1 and CAR-Tc1, respectively) in a xenograft model of mice inoculated with an aggressive human B cell lymphoma.

In this experiment we utilized a CD19-CD3ζ-CD28-41BB lentivirus vector to express a CD19-specific CAR T in Th9 cells, and compared their efficacy with a mixture of Th1 and Tc1 cells expressing the same CAR T. For the former, we cultured naïve CD4+ T cells for one day, then exposed cells to the vector and completed the standard isolation procedure for Th9 cells. For the latter, we cultured either naïve or CD4+ and CD8+ T cells with IL-2, exposed the cells to the same vector after one day, and completed the standard maturation of Th1 and Tc1 cells, respectively. We tested the therapeutic effect of the engineered T cells against CD19+ B cell lymphoma tumors established by inoculating immune-deficient NSG mice [NOD scid gamma; strain NOD.Cg-PrkdcscidIl2rgtm1Wjl/SzJl; lacking T cells, B cells, and natural killer (NK) cells] with 1×10⁶ human Raji cells, a tumor-derived line originally cultured from a Nigerian patient with Burkitt's lymphoma (Pulvertaft. “Cytology of Burkitt's Tumour (African Lymphoma)” Lancet 1:238-240 (1964). Seven days after injection of tumor cells, the mice were inoculated with a total of 3×10⁶ CD19-specific CAR T cells, either a 1:1 mix of CAR-Th1 and CAR-Tc1 (dashed line), or 100% CAR-Th9 (dotted line). Tumor-bearing mice that received no ACT (PBS alone) succumbed rapidly to the lymphoma; none survived beyond 30 days after injection with the Raji cells (solid line). The mixture of CAR-Th1 and CAR-Tel cells showed some therapeutic benefit, with the majority of mice surviving for approximately 40 days and a small minority still alive at 80 days. Notably, the mice treated with CAR-Th9 cells showed much improved survival; 90% survived at least 40 days and 80% were still alive at 80 days.

The data reveal the discovery that CAR-Th9 cells should have greater clinical benefit than the mixture of CAR-Th1 and CAR-Tc1 already tested in human patients. Taken together with the data on murine tumor models, it can be expected that a combination cell therapy utilizing CAR-Th9 cells with an antigen-presenting cell vaccine directed to the same antigen as the CAR will prove even more potent.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims.

All publications, patent applications, patents, patent publications and other references cited herein are incorporated by reference in their entireties for the teachings relevant to the sentence and/or paragraph in which the reference is presented.

The invention is defined by the following claims, with equivalents of the claims to be included therein.

TABLE 1

| REAGENT or RESOURCE | SOURCE | IDENTIFIER |
|---|---|---|
| Antibodies | | |
| APC, e450, PE anti-mouse CD45.1 | BioLegend | 110714, 110722, |
| | | 110708 |
| APC, e450, PE anti-mouse CD45.2 | BioLegend | 109814, 109820, |
| | | 109808 |
| BV570, APC, e450 anti-mouse CD4 | BioLegend | 100541, 100412, |
| | | 100428 |
| E450 anti-mouse CD44 | BioLegend | 103002 |
| PE anti-mouse CD62L | BioLegend | 104408 |
| PE anti-mouse IL-2Rβ | BioLegend | 123210 |
| PE anti-mouse CCR7 | BioLegend | 120106 |
| PE anti-mouse IL-7Rα | BioLegend | 135010 |
| PE anti-mouse Ki67 | BioLegend | 652404 |
| APC anti-mouse KLRG1 | BioLegend | 138412 |
| PE anti-mouse PD-1 | BioLegend | 135206 |
| PE anti-mouse LAG-3 | BioLegend | 125208 |
| FITC anti-mouse CD244 | BioLegend | 133504 |
| PE anti-mouse IL-9 | BioLegend | 514104 |
| APC anti-mouse IFN-γ | BioLegend | 505810 |
| PE anti-mouse IL-17A | BioLegend | 506904 |
| PE anti-mouse IL-2 | BioLegend | 503808 |
| APC anti-mouse Granzyme B | BioLegend | 372204 |
| PE anti-mouse Eomes | eBioscience | 12-4875-82 |
| Fc BLOCK | BioLegend | 101320 |
| Fixable Viability Dye eFlour 450 | eBioscience | 65-0863-14 |
| APC, FITC Annexin V | BioLegend | 640941, 640945 |
| PE anti-mouse CD8a | BioLegend | 100708 |
| PE anti-mouse Foxp3 | BioLegend | 320008 |
| anti-mouse IL-4 | BioXCell | 11B11 |
| anti-mouse IL-2 | BioXCell | JES6-1Al2 |
| anti-mouse IFNg | BioXCell | XMG1.2 |
| anti-mouse FasL | BioLegend | 106805 |
| K^{b} tetramer carrying the OVA₂₅₇₋₂₆₄ | Beckman Coulter | ts-m542-1 |
| anti-mouse p-CD3-ζ | Santa Cruz | sc-9975 |
| | Biotechnology | |
| anti-mouse TRAF6 | Santa Cruz | sc-7221 |
| | Biotechnology | |
| anti-mouse IκB-β | Santa Cruz | sc-945 |
| | Biotechnology | |
| anti-mouse p-IKKα/β (S180/181) | Santa Cruz | sc-23470-R |
| | Biotechnology | |
| anti-mouse Stat6 | Santa Cruz | sc-981 |
| | Biotechnology | |
| anti-mouse IKKγ | Santa Cruz | sc-8256 |
| | Biotechnology | |
| anti-mouse Src | Cell Signaling | No. 2108 |
| anti-mouse RelA | Cell Signaling | No. 4764 |
| anti-mouse RelB | Cell Signaling | No. 4954 |
| anti-mouse p52 | Cell Signaling | No. 4882 |
| anti-mouse NFAT1 | Cell Signaling | No. 5862 |
| anti-mouse NFAT2 | Cell Signaling | No. 8032 |
| anti-mouse HDAC1 | Cell Signaling | No. 2062 |
| anti-mouse CYLD | Cell Signaling | No. 8462 |
| anti-mouse p-TAK1 | Cell Signaling | No. 9339 |
| anti-mouse Zap70 | Cell Signaling | No. 3165 |
| anti-mouse p-Zap70 (Y319/Y352) | Cell Signaling | No. 2717 |
| anti-mouse Lck | Cell Signaling | No. 2752 |
| anti-mouse p-Lck (Y505) | Cell Signaling | No. 2751 |
| anti-mouse CD3-ζ | Cell Signaling | No. 4443 |
| anti-mouse PLCγ | Cell Signaling | No. 2822 |
| anti-mouse and β-actin | Cell Signaling | No. 4970 |
| anti-mouse p-PLCγ (Y783) | Cell Signaling | No. 14008 |
| anti-mouse p-Src (Y416) | Cell Signaling | No. 6943 |
| anti-mouse p-LAT (Y191) | Cell Signaling | No. 3584 |
| anti-mouse p-IκB-α (S32) | Cell Signaling | No. 2859 |
| anti-mouse p50 | eBioscience | 14-6732-81 |
| anti-mouse c-Rel | eBioscience | 14-6111-82 |
| Critical Commercial Assays | | |
| Foxp3 staining kit | BioLegend | 136803 |
| Annexin V-FITC Apoptosis | eBioscience | BMS500FI-100 |
| Detection Kit | | |
| NE-PER Nuclear and Cytoplasmic | ThermoScientific | 78833 |
| Extraction kit | | |
| ELISA kits mouse GM-CSF | eBioscience | 50-173-42 |
| ELISA kits mouse granzym B | eBioscience | 50-174-75 |
| ELISA kits mouse IL-9 | eBioscience | 50-112-5217 |
| ELISA kits mouse IL-10 | eBioscience | 50-112-8654 |
| ELISA kits mouse IL-6 | eBioscience | 50-112-8808 |
| ELISA kits mouse IL-21 | eBioscience | 50-174-80 |
| ELISA kits mouse TNF-α | eBioscience | 50-112-8899 |
| ELISA kits mouse IFN-γ | eBioscience | 50-112-9023 |
| ELISA kits mouse granzyme A | MyBioSource | MBS704766 |
| ChIP assay kit | Millipore | 17-295 |
| Secrete-Pair Dual Luminescence | GeneCopoeia | LF032 |
| Assay Kit | | |
| Recombinant DNA | | |
| MSCV-PIG-Pu.1 | Addgene | 66982 |
| MSCV-PIG | Addgene | 18751 |
| pLKO.1-GFP-Pu.1 shRNA | Sigma | N/A |
| pLKO.1-GFP | Addgene | 30323 |
| Negative control clone | Genecopoeia | NEG-PG04 |
| pcDNA3.1 | Qing Yi | N/A |
| pcDNA 3.1_Stat6 | Qing Yi | N/A |
| pcDNA3.1_Stat5 | Qing Yi | N/A |
| pcDNA3.1_Stat3 | Qing Yi | N/A |
| pcDNA3.1_Pu.1 | Qing Yi | N/A |
| pcDNA3.1_p50 | Qing Yi | N/A |
| pcDNA3.1_p52 | Qing Yi | N/A |
| pcDNA3.1_RelA | Qing Yi | N/A |
| pcDNA3.1_RelB | Qing Yi | N/A |
| pcDNA3.1_c-Rel | Qing Yi | N/A |
| Recombinant Proteins | | |
| Mouse GM-CSF | R&D Systems | Q14AD9 |
| Mouse TNF-α | R&D Systems | P06804 |
| Mouse IL-1β | R&D Systems | NP_032387 |
| Mouse IL-4 | R&D Systems | P07750 |
| Mouse IL-6 | R&D Systems | P08505 |
| Mouse IL-2 | R&D Systems | P04351 |
| Mouse IL-12 | R&D Systems | P43432 |
| Mouse IL-23 | R&D Systems | P43432 |
| Mouse IL-21 | R&D Systems | Q9ES17.1 |
| Human TGF-β1 | R&D Systems | P01137 |
| Oligonucleotides | | |
| mTbx21 | Sigma | N/A |
| 5′-CAACAACCCCTTTGCCAAAG-3′ | | |
| (SEQ ID NO: 17) | | |
| 5′-TCCCCCAAGCAGTTGACAGT-3′ | | |
| (SEQID NO: 18) | | |
| mEomes | Sigma | N/A |
| 5′-TTCCGGGACAACTACGATTCA-3′ | | |
| (SEQ ID NO: 19) | | |
| 5′-ACGCCGTACCGACCTCC-3′ | | |
| (SEQ ID NO: 20) | | |
| mGzmA | Sigma | N/A |
| 5′-CCTGAAGGAGGCTGTGAAAG-3′ | | |
| (SEQ ID NO: 21) | | |
| 5′-GTTACAGTGGGCAGCAGTCA-3′ | | |
| (SEQ ID NO: 22) | | |
| mGrzB | Sigma | N/A |
| 5′-AGGGGGTACAAGGTCACAGA-3′ | | |
| (SEQ ID NO: 23) | | |
| 5′-CAAGAGTGTTGTCCTTGCTCTCT-3′ | | |
| (SEQ ID NO: 24) | | |
| mGzmD | Sigma | N/A |
| 5′-TAACGAATGCCATGTAGGGG-3′ | | |
| (SEQ ID NO: 25) | | |
| 5′-TGACCCTACTTCTGCCTCTCA-3′ | | |
| (SEQ ID NO: 26) | | |
| mGzmK | Sigma | N/A |
| 5′-CCGTGGTTTTAGGAGCACAT-3′ | | |
| (SEQ ID NO: 27) | | |
| 5′-TTTTTGGATCCCAGGTGAAG-3′ | | |
| (SEQ ID NO: 28) | | |
| mPrdm1 | Sigma | N/A |
| 5′-GACAGAGGCCGAGTTTGAAG-3′ | | |
| (SEQ ID NO: 29) | | |
| 5′-GGCATTCTTGGGAACTGTGT-3′ | | |
| (SEQ ID NO: 30) | | |
| mKlrg1 | Sigma | N/A |
| 5′-CCTCTGGACGAGGAATGGTA-3′ | | |
| (SEQ ID NO: 31) | | |
| 5′-ACCTCCAGCCATCAATGTTC-3′ | | |
| (SEQ ID NO: 32) | | |
| mKlrd1 | Sigma | N/A |
| 5′-CTATGGGAGGATGGCACAGT-3′ | | |
| (SEQ ID NO: 33) | | |
| 5′-CCGTGGACCTTCCTTGTCTA-3′ | | |
| (SEQ ID NO: 34) | | |
| mKlra10 | Sigma | N/A |
| 5′-CCATAACTGCAGCAACATGC-3′ | | |
| (SEQ ID NO: 35) | | |
| 5′-ATTTAACACCTCCGCCTGTG-3′ | | |
| (SEQ ID NO: 36) | | |
| mKlrk1 | Sigma | N/A |
| 5′-CACCTTGATTTCCTCCCAGA-3′ | | |
| (SEQ ID NO: 37) | | |
| 5′-GGAAGTGAGGCAAGAACTG-3′ | | |
| (SEQ ID NO: 38) | | |
| mPrf1 | Sigma | N/A |
| 5′-AATATCAATAACGACTGGCGTGT-3′ | | |
| (SEQ ID NO: 39) | | |
| 5′-CATGTTTGCCTCTGGCCTA-3′ | | |
| (SEQ ID NO: 40) | | |
| mFasl | Sigma | N/A |
| 5′-CATCACAACCACTCCCACTG-3′ | | |
| (SEQ ID NO: 41) | | |
| 5′-TACTGGGGTTGGCTATTTGC-3′ | | |
| (SEQ ID NO: 42) | | |
| mLag3 | Sigma | N/A |
| 5′-GCCATCTCGTTCTCGTTCTC-3′ | | |
| (SEQ ID NO: 43) | | |
| 5′-GTCTCCAGTTCTCGCTCCAG-3′ | | |
| (SEQ ID NO: 44) | | |
| mPdcd1 | Sigma | N/A |
| 5′-GGAGCAGAGCTCGTGGTAAC-3′ | | |
| (SEQ ID NO: 45) | | |
| 5′-GCTCCTCCTTCAGAGTGTCG-3′ | | |
| (SEQ ID NO: 46) | | |
| mZeb2 | Sigma | N/A |
| 5′-CCACCAGCCCTTTAGGTGTA-3′ | | |
| (SEQ ID NO: 47) | | |
| 5′-CCCTTGTTCTTCTGGCTGAG-3′ | | |
| (SEQ ID NO: 48) | | |
| mSel1 | Sigma | N/A |
| 5′-ACCCACTCTCTTGGAGCTGA-3′ | | |
| (SEQ ID NO: 49) | | |
| 5′-GTTGGGCAAGTTAAGGAGCA-3′ | | |
| (SEQ ID NO: 50) | | |
| mCcr7 | Sigma | N/A |
| 5′-AGTCTTCCAGCTGCCCTACA-3′ | | |
| (SEQ ID NO: 51) | | |
| 5′-CAGCCCAAGTCCTTGAAGAG-3′ | | |
| (SEQ ID NO: 52) | | |
| mVax2 | Sigma | N/A |
| 5′-TTGGTTGACCCCAGAAACTC-3′ | | |
| (SEQ ID NO: 53) | | |
| 5′-CAAGTGTCACACAGGGATGG-3′ | | |
| (SEQ ID NO: 54) | | |
| mDapl1 | Sigma | N/A |
| 5′-CGAAAAAGACAGGCTTGGAG-3′ | | |
| (SEQ ID NO: 55) | | |
| 5′-TGGCTGTGTTTTCTGTCCTG-3′ | | |
| (SEQ ID NO: 56) | | |
| mSox2 | Sigma | N/A |
| 5′-CACAACTCGGAGATCAGCAA-3′ | | |
| (SEQ ID NO: 57) | | |
| 5′-CTCCGGGAAGCGTGTACTTA-3′ | | |
| (SEQ ID NO: 58) | | |
| mNanog | Sigma | N/A |
| 5′-AAGCAGAAGATGCGGACTGT-3′ | | |
| (SE ID NO: 59) | | |
| 5′-GTGCTGAGCCCTTCTGAATC-3′ | | |
| (SEQ ID NO: 60) | | |
| mTcf7 | Sigma | N/A |
| 5′-GCCAGAAGCAAGGAGTTCAC-3′ | | |
| (SEQ ID NO: 61) | | |
| 5′-TACACCAGATCCCAGCATCA-3′ | | |
| (SEQ ID NO: 62) | | |
| mLef1 | Sigma | N/A |
| 5′-TCACTGTCAGGCGACACTTC-3′ | | |
| (SEQ ID NO: 63) | | |
| 5′-TGAGGCTTCACGTGCATTAG-3′ | | |
| (SEQ ID NO: 64) | | |
| mTraf6 | Sigma | N/A |
| 5′-GATCGGGTTGTGTGTGTCTG-3′ | | |
| (SEQ ID NO: 65) | | |
| 5′-AGACACCCCAGCAGCTAAGA-3′ | | |
| (SEQ ID NO: 66) | | |
| mIL17a | Sigma | N/A |
| 5′-TCCAGAAGGCCCTCAGACTA-3′ | | |
| (SEQ ID NO: 67) | | |
| 5′-AGCATCTTCTCGACCCTGAA-3′ | | |
| (SEQ ID NO: 68) | | |
| Mgapdh | Sigma | N/A |
| 5′-AGCTTGTCATCAACGGGAAG-3′ | | |
| (SEQ ID NO: 69) | | |
| 5′-TTTGATGTTAGTGGGGTCTCG-3′ | | |
| (SEQ ID NO: 70) | | |
| Pu.1 binding site at the Traf6 | Sigma | N/A |
| promoter region | | |
| 5′-CTCTCCCGTGACAATGTTGGA-3′ | | |
| (SEQ ID NO: 1) | | |
| 5′-CTCCACGCTGAAGCCTTACC-3′ | | |
| (SEQ ID NO: 2) | | |
| 5′-TGTTGGAGAATGGGATCATGC-3′ | | |
| (SEQ ID NO: 3) | | |
| 5′-CTCGCTAGGAGCAGCAAGG-3′ | | |
| (SEQ ID NO: 4) | | |
| chromatin modification status of | Sigma | N/A |
| mouse Traf6 promoter | | |
| 5′-GGAGGGGACAGCTATACGCA-3′ | | |
| (SEQ ID NO: 5) | | |
| 5′-TGTGTGCTCATCACGCAGTT-3′ | | |
| (SEQ ID NO: 6) | | |
| 5′-AGCTCTCCCGTGACAATGTT-3′ | | |
| (SEQ ID NO: 7) | | |
| 5′-TTCCTCGGACCAGTGCAAAA-3′ | | |
| (SEQ ID NO: 8) | | |
| 5′-TCTACTTACCTTACCTAACAGCCT-3′ | | |
| (SEQ ID NO: 9) | | |
| 5′-GCACAATGCAATAGATGCCCA-3′ | | |
| (SEQ ID NO: 10) | | |
| chromatin modification status of | Sigma | N/A |
| mouse Traf6 enhancer | | |
| 5′-AAGGGACTCACCAAGAACCT-3′ | | |
| (SEQ ID NO: 11) | | |
| 5′-GCTCCAAATACAAGAGCAGCC-3′ | | |
| (SEQ ID NO: 12) | | |
| 5′- TACTGACTGCTGTGTTAGCTGGAA-3′ | | |
| (SEQ ID NO: 13) | | |
| 5′-GCAGAGATGCACTGTTCCCT-3′ | | |
| (SEQ ID NO: 14) | | |
| 5′- TGGACAGGGGCACTAAGACT-3′ | | |
| (SEQ ID NO: 15) | | |
| 5′-GAGCTCTGGGCTGTCTCTTC-3′ | | |
| (SEQ ID NO: 16) | | |
| Experimental Models: Organisms/ | | |
| Strains | | |
| C57BL/6 | Jackson Laboratories | 000664 |
| Cd8a^{—/—} (B6.129S2-Cd8a^{tm1Mak}/J) | Jackson Laboratories | 002665 |
| Stat6^{—/—} (B6.129S2(C)-Stat6^{tm1Gru}/J) | Jackson Laboratories | 005977 |
| Cd4-Cre (Tg(Cd4-cre)1Cwi/BfluJ) | Jackson Laboratories | 017336 |
| OT-II (B6.Cg-Tg(TcraTcrb)425Cbn/J) | Jackson Laboratories | 004194 |
| TRP-1 (B6.Cg-Rag1^{tm1Mom} Tyrp1^{B-} | Jackson Laboratories | 008684 |
| ^{w}Tg(Tcra,Tcrb)9Rest/J) mice | | |
| CD45.1 (B6.SJL-Ptprca Pepcb/BoyJ), | Jackson Laboratories | 002014 |
| Eomes^{fl/fl} (B6.129S1(Cg)- | Jackson Laboratories | 017293 |
| Eomes^{tm1.1Bflu}/J) | | |
| Ifng^{—/—} (B6.129S7-Ifng^{tm1Ts}/J), | Jackson Laboratories | 002287 |
| CD45.1 OT-II mice | Qing Yi | N/A |
| Eomes^{fl/fl} Cd4-Cre OT-II mice | Qing Yi | N/A |
| Traf6^{fl/fl} Cd4-Cre OT-II mice | Qing Yi | N/A |
| Il9r^{fl/fl} CD45.1 OT-II mice | Qing Yi | N/A |
| Il9^{fl/fl} CD45.1 OT-II mice | Qing Yi | N/A |
| Ifng^{—/—} CD45.1 OT-II mice | Qing Yi | N/A |
| Other | | |
| The NFκB-specific inhibitor QNZ | Santa Cruz | sc-200675 |
| | Biotechnology | |
| Granzyme B specific inhibitor | Enzo Life Sciences | BML-P165-0001 |
| Z-AAD-CMK | | |
| 3,4 Dichloroisocoumarin (DCI), | Santa Cruz | sc-3502 |
| inhibits granzymes A, B, and H | Biotechnology. | |
| MHC class II-restricted TRP1 | GenScript | N/A |
| (SGHNCGTCRPGWRGAACNQKILTVR) | | |
| (SEQ ID NO: 71) | | |
| MHC class II-restricted OT-II | GenScript | N/A |
| (ISQAVHAAHAEINEAGR) | | |
| (SEQ ID NO: 72) | | |

TABLE 2
Transcription factor binding sites that were
identified in the mouse Traf6 promoter

| Transcription | | Matrix | |
|---|---|---|---|
| Factor | Core Score | Score | Sequence |
| Pax-2 | 1 | 0.999 | agaAAACTt (SEQ ID |
| | | | NO: 73) |
| | | | gttAAACTg (SEQ ID |
| | | | NO: 74) |
| Myb | 1 | 0.962 | ttaAACTGag (SEQ ID |
| | | | NO: 75) |
| | | | tgtgcCAGTTa (SEQ ID |
| | | | NO: 76) |
| Smad3/4 | 1 | 0.974 | AGACAgggg (SEQ ID |
| | | | NO: 77), |
| | | | accaCAGACag (SEQ |
| | | | ID NO: 78) |
| Jun | 1 | 0.963 | agatgAGTCAaat (SEQ |
| | | | ID NO: 79) |
| Maf | 1 | 0.963 | agatgAGTCAa (SEQ |
| | | | ID NO: 80) |
| Oct1 | 1 | 0.986 | tattTGCATc (SEQ ID |
| | | | NO: 81), |
| | | | ataTTTGCatc (SEQ ID |
| | | | NO: 82) |
| Stat6 | 1 | 0.965 | aaGAAATg (SEQ ID |
| | | | NO: 83) |
| | | | aGGAAGgg (SEQ ID |
| | | | NO: 84) |
| Stat3 | 1 | 0.984 | aaGAAATg (SEQ ID |
| | | | NO: 85) |
| Sp1 | 1 | 0.956 | gaagGGCGGggta (SEQ |
| | | | ID NO: 86) |
| Ets1/2 | 1 | 0.988 | gaGGAAGg (SEQ ID |
| | | | NO: 87) |
| Gata1 | 1 | 0.991 | gcaGATGGca (SEQ ID |
| | | | NO: 88), |
| | | | tacgtGATTAatgt (SEQ |
| | | | ID NO: 89) |
| Ahr | 1 | 0.972 | aGCGTGgag (SEQ ID |
| | | | NO: 90) |
| Spi1 | 1 | 0.957 | cgaGGAAG (SEQ ID |
| | | | NO: 91) |
| E2f | 1 | 1 | GGCGCg (SEQ ID |
| | | | NO: 92) |
| Maz | 1 | 1 | ccCTCCCc (SEQ ID |
| | | | NO: 93) |
| Usf | 1 | 0.95 | ctccCGTGAc (SEQ ID |
| | | | NO: 94) |
| Hes1 | 1 | 0.982 | cggccCACGAgccgg |
| | | | (SEQ ID NO: 95) |
| Bach2 | 1 | 0.968 | gaTGAGTcaaa (SEQ |
| | | | ID NO: 96) |
| Stat5a | 1 | 0.998 | aAGAAAtg (SEQ ID |
| | | | NO: 97) |
| | | | gAGAAAac (SEQ ID |
| | | | NO: 98) |

That which is claimed is:

1. A method of treating cancer in a subject in need thereof, the method comprising:
administering to the subject an effective amount of a CD4⁺ Th9 cell that has specificity for cancer cells in the subject; and
administering a vaccine to the subject (e.g., a dendritic cell (DC) vaccine).

2. A method of reducing/eradicating a tumor in a subject in need thereof, the method comprising:
administering to the subject an effective amount of a CD4⁺ Th9 cell that has specificity for the tumor in the subject; and
administering a vaccine to the subject (e.g., a dendritic cell (DC) vaccine).

3. The method of claim 1 or 2, wherein the subject has been or is concurrently being administered an agent to induce lymphopenia.

4. The method of any of claims 1-3, wherein administering the vaccine to the subject comprises administering to the subject an effective amount of a cancer antigen-loaded antigen presenting cell (APC), wherein the cancer antigen is specific to the cancer cells in the subject.

5. The method of any of claims 1-4, wherein the CD4⁺ Th9 cell has been genetically engineered to produce a chimeric antigen receptor (CAR) that is exposed on the Th9 cell surface, wherein the CAR is specific for cancer cells in the subject.

6. The method of any of claims 1-4, wherein the CD4⁺ Th9 cell has been primed with cancer antigen-loaded APCs to have specificity for cancer cells in the subject.

7. The method of any preceding claim, wherein the cancer antigen is NY-ESO-1, WT-1, MART-1, gp100, gp75, MAGEA3, MAGEA4, HPV16-E6, Thyroglobulin, Melanoma antigen tyrosinase, CD19, CD22, CD23, CD5, CD30, CD70, CD38, CD138, CD20, CD123, HER2, IL13Ra2, CSPG4, EGFR, EGFRvIII, Mesothelin, Prostate-specific membrane antigen, CEA (Carcinoembryonic antigen), GD2 (Disialoganglioside 2), GPC3 (Glypican-3), CAIX (Carbonic anhydrase IX), L1-CAM (L1 cell adhesion molecule), CA125 (Cancer antigen 125, also known as MUC16), CD133 (prominin-1), FAP (Fibroblast activation protein), MUC1 (Mucin 1), FR-α (Folate receptor-α), Lewis-Y, Folate receptor β, DKK1, Integrin β, members of the MAGEA family (melanoma antigen family A), e.g., MAGEA1, which comprises members of the larger family of cancer testis (CT) or cancer-germline antigen family, tumor peptides derived from cyclin B1, human cancer antigens targeted by CD4+ T cells, GAGE and BAGE antigens; hTERT; PSA; survivin; p53; mutated antigens derived from the protein products of mutated oncogenes such as KRAS, NRAS, and HRAS; new epitopes created by gene translocations and fusions such as BCR-ABL in chronic myelogenous leukemia, ETV6/AML in acute lymphoblastic leukemia, NPM/ALK in anaplastic large-cell lymphomas, and ALK in neuroblastomas, including any combinations thereof.

8. The method of any preceding claim, wherein the cancer is B cell lymphoma, T cell lymphoma, myeloma, leukemia, hematopoietic neoplasias, thymoma, lymphoma, sarcoma, lung cancer, liver cancer, non-Hodgkins lymphoma, Hodgkins lymphoma, uterine cancer, cervical cancer, endometrial cancer, adenocarcinoma, breast cancer, pancreatic cancer, colon cancer, anal cancer, renal cancer, bladder cancer, prostate cancer, ovarian cancer, primary or metastatic melanoma, squamous cell carcinoma, basal cell carcinoma, brain cancer, angiosarcoma, hemangiosarcoma, head and neck carcinoma, thyroid carcinoma, soft tissue sarcoma, bone sarcoma, testicular cancer, gastrointestinal cancer, stomach cancer, glioblastoma, small cell lung cancer, non-small cell lung cancer and any combination thereof.

9. The method of any preceding claim, further comprising the steps of administering to the subject one or more chemotherapeutic agents, immunomodulatory agents, ani-inflammatory agents, immunocheckpoint blockade agents, such as PD-1, CTLA-4, PD-L1, or anti-OX40 agonist mAbs, anti-GITR agonist mAbs, anti-4-1BB agonist mAbs, a surgical procedure and/or radiation, singly or in any combination

10. A method of producing a T cell having a hyperproliferation phenotype, comprising introducing into a memory T cell or effector T cell a heterologous nucleotide sequence that encodes Traf6 and Eomes under conditions whereby the nucleotide sequence is expressed to produce the Traf6 protein and Eomes protein in the cell.

11. A cell produced by the method of claim 10.

12. A method of treating cancer in a subject in need thereof, comprising administering to the subject an effective amount of the cell of claim 11, optionally wherein the method further comprises administering a vaccine to the subject.

13. A method of reducing/eradicating a tumor in a subject in need thereof, comprising administering to the subject an effective amount of the cell of claim 11, optionally wherein the method further comprises administering a vaccine to the subject.

14. The method of claim 12 or 13, wherein the subject has been and/or is concurrently being administered an agent to induce lymphopenia.

15. The method of any of claims 12-14, wherein administering the vaccine to the subject comprises administering to the subject an effective amount of a cancer antigen-loaded antigen presenting cell (APC), wherein the cancer antigen is specific to the cancer cells in the subject.

16. The method of any of claims 12-15, wherein the cell has been genetically engineered to produce a chimeric antigen receptor (CAR) that is exposed on the cell surface, wherein the CAR is specific for cancer cells in the subject.

17. The method of any of claims 12-15, wherein the cell has been primed to have specificity for cancer cells in the subject.

18. The method of any of claims 12-17, wherein the cancer antigen is NY-ESO-1, WT-1, MART-1, gp100, gp75, MAGEA3, MAGEA4, HPV16-E6, Thyroglobulin, Melanoma antigen tyrosinase, CD19, CD22, CD23, CD5, CD30, CD70, CD38, CD138, CD20, CD123, HER2, IL13R2, CSPG4, EGFR, EGFRvIII, Mesothelin, Prostate-specific membrane antigen, CEA (Carcinoembryonic antigen), GD2 (Disialoganglioside 2), GPC3 (Glypican-3), CAIX (Carbonic anhydrase IX), L1-CAM (L1 cell adhesion molecule), CA125 (Cancer antigen 125, also known as MUC16), CD133 (prominin-1), FAP (Fibroblast activation protein), MUC1 (Mucin 1), FR-α (Folate receptor-α), Lewis-Y, Folate receptor β, DKK1, Integrin β, members of the MAGEA family (melanoma antigen family A), e.g., MAGEA1, which comprises members of the larger family of cancer testis (CT) or cancer-germline antigen family, tumor peptides derived from cyclin B1, human cancer antigens targeted by CD4+ T cells, GAGE and BAGE antigens; hTERT; PSA; survivin; p53; mutated antigens derived from the protein products of mutated oncogenes such as KRAS, NRAS, and HRAS; new epitopes created by gene translocations and fusions such as BCR-ABL in chronic myelogenous leukemia, ETV6/AML in acute lymphoblastic leukemia, NPM/ALK in anaplastic large-cell lymphomas, and ALK in neuroblastomas, including any combinations thereof.

19. The method of any of claims 12-17, wherein the cancer is B cell lymphoma, T cell lymphoma, myeloma, leukemia, hematopoietic neoplasias, thymoma, lymphoma, sarcoma, lung cancer, liver cancer, non-Hodgkins lymphoma, Hodgkins lymphoma, uterine cancer, cervical cancer, endometrial cancer, adenocarcinoma, breast cancer, pancreatic cancer, colon cancer, anal cancer, renal cancer, bladder cancer, prostate cancer, ovarian cancer, primary or metastatic melanoma, squamous cell carcinoma, basal cell carcinoma, brain cancer, angiosarcoma, hemangiosarcoma, head and neck carcinoma, thyroid carcinoma, soft tissue sarcoma, bone sarcoma, testicular cancer, gastrointestinal cancer, stomach cancer, glioblastoma, small cell lung cancer, non-small cell lung cancer and any combination thereof.

20. The method of any of claims 12-19, further comprising the steps of administering to the subject one or more chemotherapeutic agents, immunomodulatory agents, ani-inflammatory agents, immunocheckpoint blockade agents, such as PD-1, CTLA-4, PD-L1, or anti-OX40 agonist mAbs, anti-GITR agonist mAbs, anti-4-1BB agonist mAbs, a surgical procedure and/or radiation, singly or in any combination.

21. An isolated CD4⁺ Th9 cell comprising a nucleotide sequence that encodes Traf6 and Eomes whereby the nucleotide sequence is expressed to produce the Traf6 protein and Eomes protein in the cell,
wherein the CD4⁺ Th9 cell has the phenotype of a mature effector T cell.

22. The isolated CD4⁺ Th9 cell of claim 21, wherein the CD4⁺ Th9 cell has specificity for a cancer cell.

23. The isolated CD4⁺ Th9 cell of claim 21 or 22, wherein the CD4⁺ Th9 cell encodes at least one of Id2, Eomes, Id3, Il2, Gzma, Gzmb, Gzmd, Gzme, Gzmk, Gzmg, and/or Gzmn and expresses the at least one of Id2, Eomes, Id3, Il2, Gzma, Gzmb, Gzmd, Gzme, Gzmk, Gzmg, and/or Gzmn in an amount that is increased (e.g., by at least about 10% 20%, 30% 40%, 50%, or more) compared to expression level of the same gene in a Th1 and/or Th17 cell.

24. The isolated CD4⁺ Th9 cell of any one of claims 21-23, wherein the CD4⁺ Th9 cell has cytolytic activity as strong as a Th1 cell, optionally wherein the CD4⁺ Th9 cell has cytolytic activity that persists as long as a Th17 cell's in vivo.

25. The isolated CD4⁺ Th9 cell of any one of claims 21-24, wherein the CD4⁺ Th9 cell is a Ki67⁺ cell.

26. A plurality of isolated CD4⁺ Th9 cells of any one of claims 21-25.

27. The plurality of isolated CD4⁺ Th9 cells of claim 26, wherein at least about 50%, 60%, 70%, 80%, 90%, or 95% of the cells in the plurality are Ki67⁺ cells.


SEQUENCE LISTING (98 sequences)

SEQ ID NO: 1 - DNA, 21 bp, Artificial
  Pu.1 binding site at the Traf6 promoter region
ctctcccgtgacaatgttgga

SEQ ID NO: 2 - DNA, 20 bp, Artificial
  Pu.1 binding site at the Traf6 promoter region
ctccacgctgaagccttacc

SEQ ID NO: 3 - DNA, 21 bp, Artificial
  Pu.1 binding site at the Traf6 promoter region
tgttggagaatgggatcatgc

SEQ ID NO: 4 - DNA, 19 bp, Artificial
  Pu.1 binding site at the Traf6 promoter region
ctcgctaggagcagcaagg

SEQ ID NO: 5 - DNA, 20 bp, Artificial
  chromatin modification status of mouse Traf6 promoter
ggaggggacagctatacgca

SEQ ID NO: 6 - DNA, 20 bp, Artificial
  chromatin modification status of mouse Traf6 promoter
tgtgtgctcatcacgcagtt

SEQ ID NO: 7 - DNA, 20 bp, Artificial
  chromatin modification status of mouse Traf6 promoter
agctctcccgtgacaatgtt

SEQ ID NO: 8 - DNA, 20 bp, Artificial
  chromatin modification status of mouse Traf6 promoter
ttcctcggaccagtgcaaaa

SEQ ID NO: 9 - DNA, 24 bp, Artificial
  chromatin modification status of mouse Traf6 promoter
tctacttaccttacctaacagcct

SEQ ID NO: 10 - DNA, 21 bp, Artificial
  chromatin modification status of mouse Traf6 promoter
gcacaatgcaatagatgccca

SEQ ID NO: 11 - DNA, 20 bp, Artificial
  chromatin modification status of mouse Traf6 enhancer
aagggactcaccaagaacct

SEQ ID NO: 12 - DNA, 21 bp, Artificial
  chromatin modification status of mouse Traf6 enhancer
gctccaaatacaagagcagcc

SEQ ID NO: 13 - DNA, 24 bp, Artificial
  chromatin modification status of mouse Traf6 enhancer
tactgactgctgtgttagctggaa

SEQ ID NO: 14 - DNA, 20 bp, Artificial
  chromatin modification status of mouse Traf6 enhancer
gcagagatgcactgttccct

SEQ ID NO: 15 - DNA, 20 bp, Artificial
  chromatin modification status of mouse Traf6 enhancer
tggacaggggcactaagact

SEQ ID NO: 16 - DNA, 20 bp, Artificial
  chromatin modification status of mouse Traf6 enhancer
gagctctgggctgtctcttc

SEQ ID NO: 17 - DNA, 20 bp, Artificial
  mTbx21
caacaacccctttgccaaag

SEQ ID NO: 18 - DNA, 20 bp, Artificial
  mTbx21
tcccccaagcagttgacagt

SEQ ID NO: 19 - DNA, 21 bp, Artificial
  mEomes
ttccgggacaactacgattca

SEQ ID NO: 20 - DNA, 17 bp, Artificial
  mEomes
acgccgtaccgacctcc

SEQ ID NO: 21 - DNA, 20 bp, Artificial
  mGzmA
cctgaaggaggctgtgaaag

SEQ ID NO: 22 - DNA, 20 bp, Artificial
  mGzmA
gttacagtgggcagcagtca

SEQ ID NO: 23 - DNA, 20 bp, Artificial
  mGrzB
agggggtacaaggtcacaga

SEQ ID NO: 24 - DNA, 23 bp, Artificial
  mGrzB
caagagtgttgtccttgctctct

SEQ ID NO: 25 - DNA, 20 bp, Artificial
  mGzmD
taacgaatgccatgtagggg

SEQ ID NO: 26 - DNA, 21 bp, Artificial
  mGzmD
tgaccctacttctgcctctca

SEQ ID NO: 27 - DNA, 20 bp, Artificial
  mGzmK
ccgtggttttaggagcacat

SEQ ID NO: 28 - DNA, 20 bp, Artificial
  mGzmK
tttttggatcccaggtgaag

SEQ ID NO: 29 - DNA, 20 bp, Artificial
  mPrdm1
gacagaggccgagtttgaag

SEQ ID NO: 30 - DNA, 20 bp, Artificial
  mPrdm1
ggcattcttgggaactgtgt

SEQ ID NO: 31 - DNA, 20 bp, Artificial
  mKlrg1
cctctggacgaggaatggta

SEQ ID NO: 32 - DNA, 20 bp, Artificial
  mKlrg1
acctccagccatcaatgttc

SEQ ID NO: 33 - DNA, 20 bp, Artificial
  mKlrd1
ctatgggaggatggcacagt

SEQ ID NO: 34 - DNA, 20 bp, Artificial
  mKlrd1
ccgtggaccttccttgtcta

SEQ ID NO: 35 - DNA, 20 bp, Artificial
  mKlra10
ccataactgcagcaacatgc

SEQ ID NO: 36 - DNA, 20 bp, Artificial
  mKlra10
atttaacacctccgcctgtg

SEQ ID NO: 37 - DNA, 20 bp, Artificial
  mKlrk1
caccttgatttcctcccaga

SEQ ID NO: 38 - DNA, 19 bp, Artificial
  mKlrk1
ggaagtgaggcaagaactg

SEQ ID NO: 39 - DNA, 23 bp, Artificial
  mPrf1
aatatcaataacgactggcgtgt

SEQ ID NO: 40 - DNA, 19 bp, Artificial
  mPrf1
catgtttgcctctggccta

SEQ ID NO: 41 - DNA, 20 bp, Artificial
  mFasl
catcacaaccactcccactg

SEQ ID NO: 42 - DNA, 20 bp, Artificial
  mFasl
tactggggttggctatttgc

SEQ ID NO: 43 - DNA, 20 bp, Artificial
  mLag3
gccatctcgttctcgttctc

SEQ ID NO: 44 - DNA, 20 bp, Artificial
  mLag3
gtctccagttctcgctccag

SEQ ID NO: 45 - DNA, 20 bp, Artificial
  mPdcd1
ggagcagagctcgtggtaac

SEQ ID NO: 46 - DNA, 20 bp, Artificial
  mPdcd1
gctcctccttcagagtgtcg

SEQ ID NO: 47 - DNA, 20 bp, Artificial
  mZeb2
ccaccagccctttaggtgta

SEQ ID NO: 48 - DNA, 20 bp, Artificial
  mZeb2
cccttgttcttctggctgag

SEQ ID NO: 49 - DNA, 20 bp, Artificial
  mSell
acccactctcttggagctga

SEQ ID NO: 50 - DNA, 20 bp, Artificial
  mSell
gttgggcaagttaaggagca

SEQ ID NO: 51 - DNA, 20 bp, Artificial
  mCcr7
agtcttccagctgccctaca

SEQ ID NO: 52 - DNA, 20 bp, Artificial
  mCcr7
cagcccaagtccttgaagag

SEQ ID NO: 53 - DNA, 20 bp, Artificial
  mVax2
ttggttgaccccagaaactc

SEQ ID NO: 54 - DNA, 20 bp, Artificial
  mVax2
caagtgtcacacagggatgg

SEQ ID NO: 55 - DNA, 20 bp, Artificial
  mDapl1
cgaaaaagacaggcttggag

SEQ ID NO: 56 - DNA, 20 bp, Artificial
  mDapl1
tggctgtgttttctgtcctg

SEQ ID NO: 57 - DNA, 20 bp, Artificial
  mSox2
cacaactcggagatcagcaa

SEQ ID NO: 58 - DNA, 20 bp, Artificial
  mSox2
ctccgggaagcgtgtactta

SEQ ID NO: 59 - DNA, 20 bp, Artificial
  mNanog
aagcagaagatgcggactgt

SEQ ID NO: 60 - DNA, 20 bp, Artificial
  mNanog
gtgctgagcccttctgaatc

SEQ ID NO: 61 - DNA, 20 bp, Artificial
  mTcf7
gccagaagcaaggagttcac

SEQ ID NO: 62 - DNA, 20 bp, Artificial
  mTcf7
tacaccagatcccagcatca

SEQ ID NO: 63 - DNA, 20 bp, Artificial
  mLef1
tcactgtcaggcgacacttc

SEQ ID NO: 64 - DNA, 20 bp, Artificial
  mLef1
tgaggcttcacgtgcattag

SEQ ID NO: 65 - DNA, 20 bp, Artificial
  mTraf6
gatcgggttgtgtgtgtctg

SEQ ID NO: 66 - DNA, 20 bp, Artificial
  mTraf6
agacaccccagcagctaaga

SEQ ID NO: 67 - DNA, 20 bp, Artificial
  mIL17a
tccagaaggccctcagacta

SEQ ID NO: 68 - DNA, 20 bp, Artificial
  mIL17a
agcatcttctcgaccctgaa

SEQ ID NO: 69 - DNA, 20 bp, Artificial
  mGAPDH
agcttgtcatcaacgggaag

SEQ ID NO: 70 - DNA, 21 bp, Artificial
  mGAPDH
tttgatgttagtggggtctcg

SEQ ID NO: 71 - protein, 25 aa, Artificial
  MHC class II-restricted TRP1
SGHNCGTCRPGWRGAACNQKILTVR

SEQ ID NO: 72 - protein, 17 aa, Artificial
  MHC class II-restricted OT-II
ISQAVHAAHAEINEAGR

SEQ ID NO: 73 - DNA, 9 bp, Artificial
  Transcription factor binding site Pax-2
agaaaactt

SEQ ID NO: 74 - DNA, 9 bp, Artificial
  Transcription factor binding site Pax-2
gttaaactg

SEQ ID NO: 75 - DNA, 10 bp, Artificial
  Transcription factor binding site Myb
ttaaactgag

SEQ ID NO: 76 - DNA, 11 bp, Artificial
  Transcription factor binding site Myb
tgtgccagtta

SEQ ID NO: 77 - DNA, 9 bp, Artificial
  Transcription factor binding site Smad3/4
agacagggg

SEQ ID NO: 78 - DNA, 11 bp, Artificial
  Transcription factor binding site Smad3/4
accacagacag

SEQ ID NO: 79 - DNA, 13 bp, Artificial
  Transcription factor binding site Jun
agatgagtcaaat

SEQ ID NO: 80 - DNA, 11 bp, Artificial
  Transcription factor binding site Maf
agatgagtcaa

SEQ ID NO: 81 - DNA, 10 bp, Artificial
  Transcription factor binding site Oct1
tatttgcatc

SEQ ID NO: 82 - DNA, 11 bp, Artificial
  Transcription factor binding site Oct1
atatttgcatc

SEQ ID NO: 83 - DNA, 8 bp, Artificial
  Transcription factor binding site Stat6
aagaaatg

SEQ ID NO: 84 - DNA, 8 bp, Artificial
  Transcription factor binding site Stat6
aggaaggg

SEQ ID NO: 85 - DNA, 8 bp, Artificial
  Transcription factor binding site Stat3
aagaaatg

SEQ ID NO: 86 - DNA, 13 bp, Artificial
  Transcription factor binding site Sp1
gaagggcggggta

SEQ ID NO: 87 - DNA, 8 bp, Artificial
  Transcription factor binding site Ets1/2
gaggaagg

SEQ ID NO: 88 - DNA, 10 bp, Artificial
  Transcription factor binding site Gata1
gcagatggca

SEQ ID NO: 89 - DNA, 14 bp, Artificial
  Transcription factor binding site Gata1
tacgtgattaatgt

SEQ ID NO: 90 - DNA, 9 bp, Artificial
  Transcription factor binding site Ahr
agcgtggag

SEQ ID NO: 91 - DNA, 8 bp, Artificial
  Transcription factor binding site Spi1
cgaggaag

SEQ ID NO: 92 - DNA, 6 bp, Artificial
  Transcription factor binding site E2f
ggcgcg

SEQ ID NO: 93 - DNA, 8 bp, Artificial
  Transcription factor binding site Maz
ccctcccc

SEQ ID NO: 94 - DNA, 10 bp, Artificial
  Transcription factor binding site Usf
ctcccgtgac

SEQ ID NO: 95 - DNA, 15 bp, Artificial
  Transcription factor binding site Hes1
cggcccacgagccgg

SEQ ID NO: 96 - DNA, 11 bp, Artificial
  Transcription factor binding site Bach2
gatgagtcaaa

SEQ ID NO: 97 - DNA, 8 bp, Artificial
  Transcription factor binding site Stat5a
aagaaatg

SEQ ID NO: 98 - DNA, 8 bp, Artificial
  Transcription factor binding site Stat5a
gagaaaac